(19) United States
(12) Patent Application Publication
(10) Pub. No.: US 2009/0081701 A1
(43) Pub. Date: Mar. 26, 2009

(54) METHOD OF SELECTIVE PROTEIN ENRICHMENT AND ASSOCIATED APPLICATIONS

(76) Inventor: Hui Cen, Palo Alto, CA (US)

Correspondence Address: LAW OFFICES OF ALBERT WAI-KIT CHAN, PLLC, WORLD PLAZA, SUITE 604, 141-07 20TH AVENUE, WHITESTONE, NY, 11357, US

(21) Appl. No.: 12/324,554

(22) Filed: Nov. 26, 2008

Related U.S. Application Data
(63) Continuation-in-part of application No. PCT/US2007/072947, filed on Jul. 6, 2007.
(60) Provisional application No. 60/819,990, filed on Jul. 11, 2006.

(51) Int. Cl. G01N 33/53; G01N 33/00; G01N 33/566

(52) U.S. Cl. 435/7.2; 436/86; 436/501

(57) ABSTRACT

The present invention provides methods of selective enrichment of ligands present in a biological sample. One or a plurality of receptor carriers are used to capture ligands capable of binding to receptors immobilized on the surface of the receptor carriers. Receptor carriers bound with the ligands are separated from the remaining sample and the ligands are then eluted with a ligand elution solution to result in an enriched ligand sample. The enriched ligand sample may be used for further isolation of one or more ligands of interest, or for ligand profiling using 2-D gel electrophoresis coupled with mass spectrometry, for example. Such ligand profiling may have a number of applications, such as disease diagnosis, pathogen detection and drug screening.

### CROSS-REFERENCE TO RELATED APPLICATIONS

This application is a Continuation-in-part application of PCT/US2007/072947, filed Jul. 6, 2007, which claims priority of U.S. Ser. No. 60/819,990 filed Jul. 11, 2006. The content of the preceding applications are hereby incorporated in their entirety by reference into this application.

### FIELD OF THE INVENTION

The present invention generally relates to proteomics. More specifically, it relates to selective enrichment of ligand proteins from a biological sample.

### BACKGROUND OF THE INVENTION

Following completion of the human genome project, the focus of biomedical research has been shifted from high-throughput analysis of genome sequences to functional and structural studies of proteins encoded by the genetic sequences. Major efforts are now being made to determine the total number and functions of proteins present in human proteome, and to study the expression level of each protein in various organs, tissues, body fluids, or cell types. An important goal of proteomic research is to correlate the expression and modification of certain proteins with their biological phenotypes or disease states as these proteins may serve as potential pharmaceutical targets or diagnostic markers.

Proteomic research is far more challenging than genomic research because of the diversity of proteins associated with numerous secondary structure and post-translational modifications possible. As a result, unlike genomics, for which there are genome-wide analysis tools such as gene array technology and high throughput sequencing techniques, proteomics studies generally lack proteome-wide analysis tools. A common approach used in proteomic research is the so-called protein profiling, where a sample containing a mixture of proteins is subject to an analysis that yields information on the distribution of proteins according to one or more physical or biochemical properties of the proteins. Examples of currently-used protein profiling methods include two-dimensional gel (2-D gel) electrophoresis, liquid chromatography and protein/antibody arrays. Two-D gel electrophoresis and liquid chromatography profile a protein mixture according to the size and chemical properties of the proteins in the mixture while protein/antibody arrays profile proteins according to their biochemical functionalities through the binding of antibodies spotted on the array to the counterpart proteins in the sample. Recently, more powerful protein profiling techniques have been developed by combining 2-D gel electrophoresis or liquid chromatography method with mass spectrometry to allow identification of the separated proteins. Nevertheless, these current proteomic methods can only detect around 3000 proteins from a given sample due to their resolution limitation.

It is estimated that there are over 1,500,000 distinct protein molecular entities in a complex biological sample such as human plasma and/or serum (Hachey and Chaurand, J. Reprod Immunol. 2004, 63(1):61-73) and the relative amount of individual proteins present in a sample could vary by up to 10-12 orders of magnitude (“U.S. HUPO Symposium Focuses on Proteomics” Genetic Engineering News 25 (7) April 1). Since many of the most important biological signaling molecules generally fall into the low-abundance protein category, the presence of the more abundant proteins often masks the detection of low-abundance proteins, making the study of low-abundance proteins extremely difficult by conventional proteomics methods.

Consequently, many efforts have been made to increase the detection limit by eliminating the relatively abundant proteins. For example, affinity column chromatography has been used to remove 6-12 of the most abundant proteins present in human serum prior to protein profiling analysis (Lee, Anal Biochem. 2004 Jan. 1; 324(1):1-10). However even the complete elimination of the 12 most abundance proteins will only reduce less than two order of magnitude of the protein dynamic range in serum or plasma sample. Depletion of albumin and other high-abundance proteins also results in depletion of low abundance proteins that bind to albumin or other high abundance proteins (Sahab et al., Analytical Biochemistry 2007 June; Shen & Liao, Genetic Engineering News 2006 May 26(10):28). Other strategies used with limited success to overcome masking by abundant proteins include subcellular fractionation, affinity purification, and fractionation of proteins and peptides according to their physicochemical properties (Stasykt, Proteomics. 2004 December; 4(12):3704-16; Ahmed, J Chromatogr B Analyt Technol Biomed Life Sci. 2005 Feb. 5; 815(1-2):39-50). Current enrichment methods also include enrichment of biotinylated plasma membrane proteins after biotinylating membrane proteins of intact cells (Zhang et al., Electrophoresis, 2003, 24:2855-2863) and enrichment of phosphoproteins (Saiful et al., Rapid Commun. Mass Spectrom. 2005; 19:899-909). However, enrichment efficiency with these techniques is limited considering the prevalence of both membrane proteins ( 1/10 of total cellular proteins) and phosphoproteins ( 1/10 of total cellular proteins). Therefore, these current strategies have had only some degree of success in effectively profiling relatively rare or low abundant proteins.

An efficient way to discover function-specific proteins is to isolate the proteins via a functionality-dependent technique. This approach eliminates proteins that are irrelevant to the selected protein function(s) while enriching relevant proteins for subsequent profiling study. Protein samples enriched in this manner can be more easily profiled within the resolution of conventional methods since the number of proteins has been dramatically decreased. For example, proteins that function as ligands, receptors or other binding proteins have been isolated by affinity purification, wherein either a known ligand or a known receptor serves as a “bait” molecule for capturing the counterpart protein molecule (Feshchenko et al., Oncogene 2004 Jun. 10, 23(27):4690-706. Erratum, Oncogene. 2004 Dec. 16, 23(58):9449). Alternatively, relevant proteins may also be isolated based on biological activity elicited upon binding between a known bait molecule and its counterpart molecule in a ligand-receptor affinity interaction scheme. (Civelli et al., FEBS Lett. 1998 Jun. 23, 430(1-2):55-8). However, this protein purification or protein enrichment method has thus far been limited to isolating target molecules whose bait molecules are known and mostly a single ligand or receptor molecule is isolated at a time.

Ligands and receptors are significant molecules in multi-cellular organism since they comprise the communication network for the organism. Many ligands have also been found to be relevant biomarkers for inflammation. To date, more than 50% of the drugs on the market are either derived from or targeted to ligands or receptors. Since ligands and receptors mostly are low abundance proteins, they tend to be missed by the current proteomics methods without enrichment.

Thus, it is desirable to develop an efficient enrichment method for low-abundance yet biologically important proteins such as ligands and receptors from a biological sample.

### SUMMARY OF THE INVENTION

The present invention provides a method for selectively enriching suitable biological molecules present in a complex system, for example a biological fluid, by using one or a plurality of receptor carriers wherein each receptor carrier comprises a plurality of receptors on its surface. The receptor carrier or carriers may be cells, sub-cellular organelles, vesicles comprising a membrane comprising a plurality of receptors, or artificial biological surface comprising a plurality of receptors. In one embodiment of the invention, the receptor carrier or carries are live or fixed cells, wherein the exterior membrane-bound receptors of the cells are capable of binding/capturing ligands present in a biological fluid sample.

The selective ligand enrichment method generally comprises the steps of: 1) exposing the liquid extract of a biological sample to a receptor carrier or carriers for a time sufficient for any suitable ligands present in the liquid extract to bind to their respective receptors on the biological surface of the receptor carrier or carriers; 2) removing unbound molecules in the liquid extract of the biological sample after ligand/receptor binding; 3) dissociating the receptor-bound ligands from the receptor carrier or carriers by using a ligand elution solution; and 4) separating the liquid containing the enriched ligands from the receptor carrier or carriers to provide an enriched ligand sample.

The enriched ligand sample may be suitable for a variety of purposes, including profiling ligands that are present in the original sample and that are relevant to the selected biological functionality of interest. Protein profiling or ligand profiling yields “finger-print” information on the mixture in terms of the composition and quantity of the ligands present in the mixture according to physical and biochemical characteristics of the ligands. Profiling of the enriched ligand sample may be conducted by use of 1-D or 2-D gel electrophoresis, chromatography, mass spectrometry or other means to separate and analyze the ligands by means of molecular weight, pI, hydrophobicity/hydrophilicity, etc.

Ligand profiling using an enriched ligand sample according to the present invention may have many practical applications, for example: mapping of ligand proteome for any organism; characterizing metabolomics and assessing health condition of an individual; identifying biomarkers for human disease diagnosis, prognosis, drug response and/or drug screening.

In one embodiment, there is provided a method of enriching multiple ligands in a sample, the method comprising: (a) contacting a sample comprising a plurality of ligand molecules with a plurality of receptor carriers, wherein each receptor carrier comprises a plurality of receptors to which the ligand molecules may bind; (b) removing unbound ligand molecules by washing; and (c) eluting bound ligand molecules from the receptor carriers to give a solution enriched with multiple ligand molecules.

In another embodiment, there is provided a method of profiling one or more receptor carrier's ligands, the method comprising: (a) contacting a sample comprising a plurality of ligand molecules with one or more receptor carriers, wherein each receptor carrier comprises a plurality of receptors to which the ligand molecules may bind; (b) removing unbound ligand molecules by washing; (c) eluting bound ligand molecules from the receptor carriers to give a ligand molecule fraction; and (d) fractionating the ligand molecule fraction to give a profile of ligand molecules that bind to the receptors of the receptor carriers.

In another embodiment, there is provided a method of differential ligand profiling between two or more distinct samples comprising mixtures of ligand molecules, the method comprising: (a) contacting each of the distinct samples with a separate populations of receptor carriers, wherein each receptor carrier comprises a plurality of receptors to which the ligand molecules may bind; (b) washing unbound ligand molecules away and eluting the bound ligand molecules from the receptor carriers to provide separate ligand fractions; (c) fractionating the ligand fractions to give separate profiles of ligand molecules that bind to the receptors of the receptor carriers; and (d) comparing the profiles obtained in (c) to give a differential ligand profile between the distinct samples.

In another embodiment, there is provided a method of profiling a cell population's polypeptide ligands, the method comprising: (a) contacting a sample comprising a plurality of polypeptide ligands to the cells, wherein the cells comprise a plurality of receptors to which the polypeptide ligands may bind; (b) removing unbound molecules by washing; (c) eluting bound polypeptide ligands from the cells to give a polypeptide ligand fraction; and (d) fractionating the polypeptide ligand fraction to give a profile of polypeptide ligands that bind to the receptors of the cells.

In another embodiment, there is provided a method of differential polypeptide ligand profiling between two or more samples comprising polypeptide ligands, the method comprising: (a) contacting each sample comprising polypeptide ligands with a separate population of cells, wherein each population of cells comprises a plurality of receptors to which the polypeptide ligands may bind; (b) washing unbound molecules away and eluting the bound polypeptide ligands from each population of cells to provide separate polypeptide ligand fractions; (c) fractionating the polypeptide ligand fractions to give separate profiles of polypeptide ligands that bind to the receptors of the cells; and (d) comparing the profiles obtained in (c) to give a differential polypeptide ligand profile between the distinct samples of polypeptide ligands.

In another embodiment, there is provided a kit for enriching multiple ligands from a sample comprising ligands with unknown identity or quantity, the kit comprising: binding solution; washing solution; an elution solution; and an instruction on experimental procedures accordingly to the methods of the present invention. The kit may further comprise a plurality of receptor carriers comprising a plurality of receptors to which the ligands may bind.

In another embodiment, there is provided a method of differential receptor profiling between two or more distinct cellular samples using the same mixture of ligands, the method comprising: (a) contacting an aliquot of the mixture of ligands with each of the cellular samples, wherein each cellular sample comprises a plurality of receptors to which the ligands may bind; (b) washing unbound ligands away and eluting the bound ligands from each of the cellular samples to provide separate ligand fractions; (c) fractionating the ligand fractions to give separate profiles of ligands that bind to the receptors of each of the cellular samples; and (d) comparing the profiles obtained in (c) to give a differential ligand profile reflecting differential receptor profile between/among the distinct cellular samples.

### BRIEF DESCRIPTION OF THE DRAWINGS

A detailed description of various aspects, features and embodiments of the present invention is provided herein with reference to the accompanying drawings, which are briefly described below. The drawings are illustrative and are not necessarily drawn to scale. The drawings illustrate various aspects, or features, of the present invention and may illustrate one or more embodiment(s) or example(s) of the present invention in whole or in part. A reference numeral, letter, and/or symbol that are used in one drawing to refer to a particular element or feature may be used in another drawing to refer to a like element or feature.

FIG. 1 is a schematic illustration of the process for selective enrichment of ligand molecules, L1, L2, L3, L4, L5 and L6, from a biological sample using a receptor carrier comprising receptor molecules, R1, R2, R3, R4, R5 and R6, on the surface of the receptor carrier. The number of ligands and the corresponding number of receptors shown in the scheme are only for illustrative purpose. Actual numbers of ligands and receptors may be any such as the normal number of ligands and receptors present in a cellular system or a biological sample. The receptor carrier may assume any physical shape such as a sphere of regular or irregular shape, a sheet of regular or irregular shape, or a rod of regular or irregular shape, merely by way of example. The receptor carrier may be a cell; an organelle; a vesicle made of everted cytoplasmic membrane, everted organelle membrane, or synthetic lipids, wherein the vesicle surface is immobilized with a plurality of receptors; or an artificial substance or object on whose surface a plurality of receptors are immobilized. The oval-shaped objects represent receptor molecules. The crescent-shaped objects represent ligand molecules while the triangle-shaped objects represent non-ligand molecules.

FIG. 2 is a schematic illustration of the process for selective enrichment of ligand molecules, L1, L2, L3, L4, L5 and L6, from a biological sample by a receptor carrier prepared through immobilizing onto the surface of a container (such as a vial) receptor molecules, R1, R2, R3, R4, R5 and R6. The number of ligands and the corresponding number of receptors shown in the scheme are only for illustrative purpose. Actual numbers of ligands and receptors may be any such as those normally present in a biological sample. The oval-shaped objects represent receptor molecules while the triangle-shaped object represents non-receptor and non-ligand molecules. The crescent-shaped objects represent ligand molecules.

FIG. 3A is 1-D Western blot image of ligands enriched from a serum sample (serum HC) by NIH3T3 and Hela cells. FIG. 3B is 1-D Western blot image of differential ligand profiling among four multiple myeloma patients (Patient #1-4) and one healthy individual (Serum HC) by specific detection of serum-derived biotin-labeled ligands (see example 7).

FIG. 4A is a fluorescent image of a 2-D electrophoresis gel for combined ligands enriched from two human plasma samples (Samples #1 and #2) using intact Hela cells as receptor carriers. To obtain the combined ligand profile from the two samples, enriched ligand sample from Sample #1 was minimally labeled with the fluorescent dye Cy3 (green pseudo color) and enriched ligand sample from Sample #2 was minimally labeled with another dye Cy5 (red pseudo color). The two labeled samples were combined in equal amount and then subject to 2-D gel electrophoresis. FIG. 4B is a fluorescent image of a 2-D gel obtained with a mixture of equal amount of human plasma labeled with Cy3 and human serum labeled with Cy5. FIG. 4B was used as a reference for FIG. 4A to demonstrate selective enrichment of a small group of proteins in human plasma by Hela cells as a receptor carrier. (See Example 8)

FIG. 5A is the same as FIG. 4A. The highlighted box in FIG. 5A is enlarged in order to see the distinct green and red spots (circled areas) representing differentially expressed ligands in the two samples, respectively (FIG. 5B).

FIG. 6 is a 1-D SDS-PAGE gel image showing different ligand profiles of the same human plasma sample as a function of the receptor carrier used for the ligand enrichment. A human plasma sample was enriched using three separate cell lines, Hela, MCF7 and Jurkat, as receptor carriers to give three separate ligand samples, LHela, LMCF7 and LJurkat, respectively. Each ligand sample was then subject to one-dimensional SDS-PAGE, giving the profiles shown in Lanes LHela, LMCF7 and Ljurkat, respectively. Lanes LCHela, LCMCF7 and LCJurkat represent profiles for proteins eluted from Hela, MCF7 and Jurkat cells, respectively, after incubation with 5 mL PBS. (See Example 9)

### DETAILED DESCRIPTION OF THE INVENTION

In relation to the brief summary and the description, it will be understood that a word appearing in the singular encompasses its plural counterpart, and a word appearing in the plural encompasses its singular counterpart, unless implicitly or explicitly understood or stated otherwise. Further, it will be understood that for any given component described herein, any of the possible candidates or alternatives listed for that component, may generally be used individually or in any combination with one another, unless implicitly or explicitly understood or stated otherwise. Additionally, it will be understood that any list of such candidates or alternatives, is merely illustrative, not limiting, unless implicitly or explicitly understood or stated otherwise. Still further, it will be understood that any figure or number or amount presented herein in connection with the invention is approximate, and that any numerical range includes the minimum number and the maximum number defining the range, unless implicitly or explicitly understood or stated otherwise. Additionally, it will be understood that any permissive, open, or open-ended language encompasses any relatively permissive to restrictive language, open to closed language, or open-ended to closed-ended language, respectively, unless implicitly or explicitly understood or stated otherwise. Merely by way of example, the word “comprising” may encompass “comprising”, “consisting essentially of”, and/or “consisting of” type language.

Various terms are generally described or used herein to facilitate understanding of the invention. It will be understood that a corresponding general description or use of these various terms applies to corresponding linguistic or grammatical variations or forms of these various terms. It will also be understood that a general description or use of a corresponding general description or use of any term herein may not apply or may not fully apply when the term is used in a non-general or more specific manner. It will also be understood that the invention is not limited to the terminology used herein, or the descriptions thereof, for the description of particular embodiments. It will further be understood that the invention is not limited to embodiments of the invention as described herein or applications of the invention as described herein, as such may vary.

Generally, the term “biological surface” refers to a surface or matrix on which a plurality of receptors are or can be immobilized either non-covalently or covalently for interaction with ligands present in a sample; and wherein the biological surface can be natural such as a whole cell, the exterior or interior surface of a cytoplasmic membrane, cell organelle membrane, a tissue, the exterior surface of a liposome or micelle, or artificial such as surface of a non-biological material wherein the material may be in the physical form of a well, a plate, a particle, a bead, a fiber, a matrix, a porous structure, a stick, a membrane, a chip, or the like, and the material may be selected from the list of sepharose, agarose, latex, dextran, lipid monolayer, lipid bilayer, metal, metal oxide, glass, ceramic, quartz, plastic, silicon, polyacrylamide, polystyrene, polyethylene, polypropylene, polymer, a colloid, polycarbonate, polytetrafluoroethylene, silicon oxide, silicon nitride, cellulose acetate membrane, nitrocellulose membrane, nylon membrane and polypropylene membrane, amorphous silicon carbide, castable oxides, polyimides, polymethylmethacrylates, and silicone elastomers and/or the like. Biological surface can also be other form besides surface and matrix as long as it can be separated from the solution containing or suspected to contain ligand molecules by conventional separation methods such as centrifugation, filtration, precipitation, magnetic field, affinity capture and the like. One example of a biological surface is the outer leaflet of cell membrane embedded with receptor proteins capable of interacting with ligands present in a biological sample. An example of an artificial biological surface is the surface of an assay well, plate or bead, or matrix within a column containing materials coated with immobilized proteins capable of interacting with ligands present in a biological sample.

Generally, the term “receptor” or “receptor molecule” refers to a protein, a protein complex, a peptide or a peptide complex, nucleic acid, metabolic product and by-product, or organic molecule(s) presented by cells or that is immobilized to a biological surface as defined above and is available for interaction with a ligand present in a solution such as a biological fluid. For example, a “receptor” may be a cell membrane receptor molecule for a growth factor or a cytokine. A “receptor” may be a truncated membrane receptor molecule only containing the extracellular domain, the ligand binding domain of the membrane receptor molecule. A “receptor” may also be an immobilized ligand protein that is capable of binding to soluble extracellular domain of the membrane receptor molecule in a solution. Alternatively, a receptor may be a membrane protein or membrane peptide that acts as an antigen for an antibody present in the sample. Conversely, a receptor may be a membrane protein or peptide that acts as an antibody against an antigen present in the sample. As non-limiting illustrative examples, an affinity chromatography matrix having multiple immobilized polypeptides, or an isolated cell membrane fraction coupled to an insoluble matrix, or an intact cell, are but a few of the embodiments of receptor-containing surfaces encompassed by the invention.

Generally, the term “receptor carrier” refers to a substance carrying a plurality of receptor molecules wherein the receptors are capable of interacting with ligand molecules in the sample. Biological surface defined above is one form of receptor carrier.

Generally, the term “ligand” refers to a protein, polypeptide, peptide, nucleic acid, metabolic product and by-product, organic or inorganic molecule present in a prepared or naturally occurring sample. For example, mixtures of known polypeptides prepared in a laboratory or industrial setting, as well as naturally occurring biological fluids or extracts of biological materials are encompassed herein as sources of “ligands”. From a functional perspective, a “ligand” is a molecule that is capable of binding to one or more sites of receptor molecules on an artificial or a naturally occurring biological surface. A “ligand” can be a growth factor, a cytokine, a soluble extracellular domain of a receptor, a soluble polypeptide or other molecule found in an organism which is capable of binding to another polypeptide or protein immobilized on a biological surface. As non-limiting illustrative examples, a protein, a peptide, a sugar/carbohydrate, a lipid, a steroid or steroid hormone, a nucleic acid are but a few of the embodiments of ligands encompassed by the invention

For the purpose of profiling the putative ligands in a sample, persons of ordinary skill in the art would appreciate that the terms “receptors” and “ligands” encompass molecules that may or may not possess a known physiological function.

Generally, the term “biomarkers” refers to a characteristic, or a combination of characteristics, that can be objectively measured and evaluated as an indicator of normal biological processes, pathogenic processes, or a pharmacological response to a therapeutic intervention.

The term “organism” refers to a single-celled organism or a multi-celled organism, wherein the multi-celled organism may be a plant species, an animal or a human. For animal, it can be invertebrate or vertebrate. Representative examples of a multi-celled organism include, but are not limited to, Bos taurus, Gallus gallus, Maleagris gallopavo, Mus musculus, Ovis ammon, Rattus norwegicus, Sus scrofa (in general: insect, worm, fish, mouse, rat, dog, cat, cow, goat, sheep, chicken, hog) and Homo sapiens.

Generally, the term “biological fluid” refers to all fluids that contain or are suspected to contain biologically relevant molecules (including, but not limited to, proteins, peptides, nucleic acids, steroids or steroid hormones, sugars/carbohydrates, lipids, other small molecules) as ligand(s) described in this invention. The biological fluid may be a solution containing multiple known or unknown ligand(s) or a mixture containing multiple known or unknown ligand(s). Typical examples of biological fluids include, but are not limited to, bodily fluids such as blood, blood plasma, blood serum, hemolysate, spinal fluid, urine, lymph, synovial fluid, saliva, semen, stool, sputum, cerebral spinal fluid, tear, mucus, amniotic fluid, lacrimal fluid, cyst fluid, sweat gland secretion, bile, milk and the like. Additional examples of “biological fluid” include medium supernatants of culture cells, tissue, bacteria and viruses as well as lysates obtained from cells, tissue, bacteria or viruses. Cells and tissue can be derived from any single-celled or multi-celled organism described above.

Generally, the term “sample” refers to all biological specimens or the derivatives of biological specimens that contain or are suspected to contain biologically relevant molecules (including, but not limited to, proteins, peptides, nucleic acids, steroids or steroid hormones, sugars, lipids, other small molecules) as ligand(s) described in this invention. The specimen may contain multiple known or unknown ligand(s) or a mixture containing multiple known or unknown ligand(s). The specimen may be a biological fluid; a tissue of a plant, fungus, animal or human origin; cell(s) of a bacterium, plant, fungus, animal or human origin; viruses and other micro-organisms; lysates; fractions or other derivatives of the biological specimens described above; or naturally occurring materials (such as water, soil, air) that contain the biological specimens described above.

Generally, the term “analytical method” refers to all laboratory methods and protocols that are used to identify, quantify, distinguish or characterize ligand molecule(s) that are enriched using the invention described herein. Analytical methods may include liquid chromatography, gas chromatography, gel electrophoresis, mass spectrometry (MS), densitometry, colorimetrics, spectrophotometry, energy magnetic radiation, nuclear magnetic resonance (NMR), and combinations thereof, just by way of example. To analyze ligands that are unknown organic small molecules, conventional methods used in analytical chemistry such as various chromatography methods can be used to separate and isolate each individual components and analyze each by a combination of MS, NMR, elemental analysis, IR, UV/Vis and the likes.

Generally, the term “proteomic method” refers to all laboratory methods and protocols that are used to identify, quantify, distinguish or characterize proteins and peptides that are enriched using the invention described herein. Some proteomic methods are described in Current Protocols in Protein Sciences, 2007, by John Wiley and Sons, Inc. Proteomic methods may include one-dimensional gel electrophoresis (1-D GE) and staining, two-dimensional gel electrophoresis (2-D GE) and staining, two-dimensional differential in-gel electrophoresis (2-D DIGE), capillary electrophoresis (CE), Western blotting analysis, ELISA, protein microarrays, reverse-phase protein microarrays, liquid chromatography, mass spectrometry, Isotope Coded Affinity Tags (ICAT), Isobaric Tags for Relative and Absolute Quantitation (iTRAQ), Stable-Isotope Labeling with Amino acids in Cell culture (SILAC), Surface Enhanced Laser Desorption/Ionization Time of Flight mass spectroscopy (SELDI-ToF), and combinations thereof, merely by way of example.

Generally, the term “unknown” proteins refer to proteins whose identity is not known to researchers before they are identified through their research. They can be novel proteins whose sequence have not been identified before or non-novel proteins whose sequence have been identified before such as IL6, VEGF and IL2. For example, a researcher set out to find differentially expressed proteins between lung cancer patients and health individuals from their sera as potential biomarkers. The researcher does not know the identity of these differentially expressed proteins at the beginning. So they are “unknown” proteins to him. The differentially expressed proteins were identified through his experiments and their identity was later identified as IL6, VEGF and a novel protein. All three proteins IL6, VEGF and the novel protein were considered “unknown” proteins in their identity in this patent application.

A method for selectively enriching biological ligands from a biological sample using a receptor carrier is now described. The enriched ligand sample obtained according to the present invention is useful for profiling ligands that are present in the original biological sample and that are specific to the receptors present on the receptor carrier. Ligand profiling may be carried out by using any of the known analytical methods or proteomics methods such as those described in the previous paragraphs. Ligand profile information may be useful for a variety of applications including, but are not limited to, identifying biomarkers for various diseases, disease staging and monitoring, and for drug screening.

It is widely known that ligand-receptor interactions are fundamental to signal transduction in multi-celled organisms. For a multi-celled organism, an external signal may be in the form of one or more ligand molecules that are carried by the organism's bodily fluids throughout the entire organism. Once the ligand or ligands are captured by the target cells that possess the corresponding receptor or receptors, characteristic cellular activities take place in response to the external signal. In a complex multi-celled organism like a human, any physiological or pathological response is most likely orchestrated by an array of ligands through binding to respective receptors on their target cells. Frequently these ligands act as biomarkers characteristic of certain disease or disease state. Biological ligands typically exist in very small quantities as compared to the relatively abundant amounts of other common proteins such as carrier proteins present in biological fluids. Thus, selective enrichment and subsequent identification of these ligands may greatly enhance the understanding of cellular function and regulation. As an example, the identification of ligands in synovial fluid for various cells in the joint may provide biologists new important information for designing new therapeutics for arthritis and biomarkers for accurate diagnosis.

Many proteomics technologies are amenable to high throughput analysis such as 2-D gel electrophoresis coupled with mass spectrometry. Subjecting selectively-enriched low abundance ligands from a biological sample to such analysis would enable one to profile the ligands present in the biological sample according to relative quantities and physical and/or biochemical properties of the ligands. For example, by comparing the ligand profiles between a sample of diseased state and a sample of non-diseased state, one may readily identify disease-associated ligands that may serve as new therapeutic targets or diagnostic biomarkers.

The present invention provides a method for selectively enriching ligands present in a biological sample by using one or a plurality of receptor carriers wherein each receptor carrier comprises a plurality of receptors on its surface. In one embodiment, the method comprises exposing a biological fluid to the receptor carrier or carriers for a time sufficient for any suitable ligands present in the fluid to bind to their respective receptors on the biological surface of the receptor carrier or carriers; removing the receptor carrier or carriers from the remaining biological fluid after ligand/receptor binding; dissociating the receptor-bound ligands from the receptor carrier or carriers by using a ligand elution solution; and separating the liquid containing the enriched ligands from the receptor carrier or carriers to provide an enriched ligand sample.

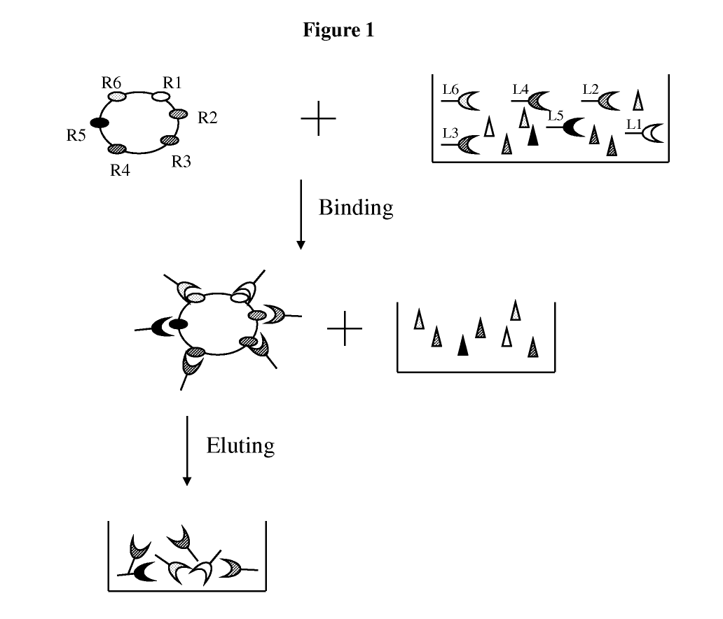

The receptor carrier or carriers may be substances or objects wherein at least part of the surface of the substances or objects is a biological surface. The receptor carrier or carriers may be cells, organelles, vesicles comprising a membrane comprising a plurality of receptors, or artificial solid substances having a biological surface comprising a plurality of receptors. The receptor carrier or carriers may be readily separated from a liquid by any known techniques such as aspiration of the liquid or centrifugation. FIG. 1 illustrates one embodiment of the ligand enrichment process of the present invention.

In one embodiment of the invention, each receptor carrier is a cell of known identity, a cell of known tissue identity, or a cell of known species identity, wherein the cell surface comprises a plurality of receptors. Preferably, the receptor carrier is a cell of known identity or a cell of known tissue identity. The cell may be a live cell, an apoptotic cell, a dead cell or a fixed cell as long as the receptors are capable of binding their ligand molecules. Cells can be fixed by a number of agents and methods currently known in the art (e.g. formaldehyde fixation). The cell may be prokaryotic or eukaryotic. The cell can be an animal cell, a plant cell, a bacteria cell, a yeast cell or a fungus cell, merely by way of example. When the cell is of animal origin, it may be a cell from any vertebrate or any invertebrate animal. Examples of vertebrate animals include, but are not limited to, humans, mice, rats, pigs, cows, monkeys, rabbits, chickens, and the likes. Examples of invertebrate animals include, but are not limited to, drosophila, zebra fish, worms and the likes. The cell may be an adherent cell such as HeLa, PC3, Cos cell or the like, or maybe a suspension cell such as Jurkat, HL-60 cell, and/or the like, merely by way of example. The cell may belong to a primary cell type or to an immortalized cell type.

In another embodiment, each receptor carrier is a cell that has been genetically engineered to express on its surface at least one receptor that is not naturally expressed to the desired quantity on the surface of the cell. Many receptors may not be expressed or may not be expressed at a sufficiently high quantity to allow efficient enrichment of the ligands that bind to these receptors. Exogenous expression of a receptor on the cell surface will allow the engineered cell to enrich the respective ligand. For example, HeLa cells normally lack surface expression of the receptor for human nerve growth factor (hNGF) (Grob et al., Proc. Natl. Acad. Sci. USA., 1983 Nov. 15 80(22):6819-6823). As a result, hNGF can not be enriched using HeLa cells. By stably transfecting HeLa cells with a vector that expresses large quantity of hNGF receptor on HeLa cell surface, the ligand enrichment capacity of HeLa cells can be extended to include hNGF.

In another embodiment, receptor carriers are a mixture of cell types. Each type of cell expresses a different set of receptors on the surface. Pooling different types of cells as receptor carriers increases the chance of having desired receptors present on one or more cell type in sufficient quantity to enrich a variety of ligands from a biological sample. For example, Hela cell expresses minimal amount of platelet derived growth factor (PDGF) receptor and high amount of epidermal growth factor (EGF) receptor while NIH3T3 cell expresses minimal amount of EGF receptor and high amount of PDGF receptor. By pooling both Hela cell and NIH3T3 cell as receptor carriers, both EGF and PDGF can be efficiently enriched from a given biological sample.

In yet another embodiment, multiple receptor genes in one or more mammalian expression vectors are introduced into a cell to allow exogenous expression of corresponding receptors on the cell surface. This will allow the engineered cells to enrich the respective ligands. The receptor genes can range from two or more, to a library of receptor genes encoding most if not all receptors whose ligands are of interest. For example, HeLa cells normally lack cell surface expression of the receptor for human nerve growth factor (hNGF), and have very low cell surface expression of the receptor for interleukin-6 (IL-6, Hess et al., J. Immunology, 2000, 165:1939-1948). By stably transfecting a HeLa cell population with vectors that express hNGF receptor and IL-6 receptor, the ligand enrichment capacity of HeLa cells can be extended to include both hNGF and IL-6. Furthermore, a library of expression vectors which collectively express a fraction or all of the known cell surface receptors can be used to transfect HeLa cells or any other cell types to expand their ligand enrichment capacity. A comprehensive listing of receptors can be found in Izhar et al., Sci. STKE 2003 (187) p. 9 and in Appendix A.

The receptor genes can be placed in a plasmid vector or integrated into a viral genome. For example, they can be introduced into the cell as the receptor carrier by transfection or viral infection such as retrovirus infection or lentivirus infection.

In another embodiment, each receptor carrier is a cell organelle comprising a plurality of receptors on the surface of the organelle, wherein the organelle can be a cell nucleus, an endoplasmic reticulum, a Golgi, a mitochondrion, a lysosome, an endosome, a peroxisome, a chloroplast, a synaptic vesicle, a clathrin-coated vesicle, a melanosome, a mass cell granule or any of the other organelles described in Current Protocols in Cell Biology, 2005 by John Wiley & Sons. The target cellular organelle may be isolated according to methods described in Current Protocols in Cell Biology, 2005 by John Wiley & Sons or elsewhere. Several commercial companies such as Sigma (St Louis, Mo.) and Biovision (Mountain View, Calif.) offer ready-to-use kits for isolating specific organelles. The same organelle pooled from different cells can be used to increase the spectrum of the enriched ligands.

In another embodiment of the invention, each receptor carrier is a cell organelle comprising a plurality of receptors capable of binding to suitable ligands present in a biological fluid on the surface of the organelle, wherein at least one of the receptors is expressed from an exogenous expression vector artificially introduced into the cell. In this embodiment, the organelle is prepared from cells that are genetically engineered to express exogenous receptors, wherein at least one of the exogenously receptors is located on the surface of the organelle.

In another embodiment of the invention, receptor carrier may comprise a vesicle whose membrane is made of an everted cytoplasmic membrane or an everted organelle membrane such that the interior surface of the cytoplasmic membrane or an organelle membrane is now the exterior surface of the vesicle. Many proteins on the interior surface of cytoplasmic membrane or organelle membrane are responsible for transmitting signals generated by ligand/receptor binding on the exterior surface of cytoplasmic membrane or organelle membrane to the cytoplasm or the interior of the cell organelle during signal transduction process (Philips, Biochem Soc Trans. 2005, 33(Pt 4):657-61). Methods for preparing vesicles with everted plasma membrane of eukaryotic and prokaryotic cells have been described by van der Meulen et al. Biochim Biophys Acta. 1981, 643(3):601-15; Kinoshita et al., J. Cell Biol. 1979, 82(3):688-96; Kalish et al., Biochim Biophys Acta. 1978, 506(1):97-110; Jacobson et al., Biochim Biophys Acta. 1978, 506(1):81-96; Cohen et al., J. Cell Biol. 1977, 75(1):119-34; Lange et al., Proc Natl Acad Sci USA. 1977, 74(4):1538-42; Jascobson et al., Science 1977, 195(4275):302-4; Harford et. al., Proc Natl Acad Sci USA. 1981, 78(3):1557-61; Hou et al., J Biol. Chem. 2000, 275(27):20280-7; Scarborough, Methods Enzymol 1989; 174:667-76. The same everted cytoplasmic membrane or everted organelle membrane pooled from different cells can be used to increase the spectrum of the enriched ligands.

In another embodiment of the invention, each receptor carrier is an everted cytoplasmic membrane or an everted organelle membrane comprising a plurality of receptors capable of binding to suitable ligands present in a biological fluid on the surface of the everted membrane, wherein at least one of the receptors is expressed from an exogenous expression vector artificially introduced into the cell. In this embodiment, the everted membrane is prepared from cells that are genetically engineered to express exogenous receptors, wherein at least one of the exogenously receptors is located on the surface of the everted membrane.

In another embodiment, the receptor carrier is cell ghost or membrane preparations. Membrane preparations can be from plasma membranes or subcellular organelle membranes. Cell ghost and membrane preparations are broken plasma membrane or organelle membrane therefore exposing a plurality of receptors on both internal and external sides of the membrane for ligand binding and enrichment. Methods of preparing cell ghost and membrane preparations can be found elsewhere such as in Current Protocols in Cell Biology, 2007, by John Wiley and Sons, Inc., and by Arthur K Parpart, Journal of Cellular and Comparative Physiology, 1965, 19(2): 248-249.

In another embodiment of the invention, the receptor carrier is an artificially made vesicle having a membrane comprising a plurality of receptors. This kind of receptor carrier can be made by employing commonly used techniques (Zawada Z. Cell Mol Biol Lett. 2004; 9(4A):603-15) for making artificial vesicle such as liposomes in the presence of desired receptor molecules.

In another embodiment, the receptor carrier or carriers may be cells grown on surfaces such as beads or microcarriers. Microcarriers can be solid or porous. Microcarriers can be made by various materials such as dextran, alginate, polyethylene, plastic, glass, metal and other materials. Cells can be grown as monolayers on the surface of microcarriers; as multilayers in the pores of the porous structure of microcarriers; or as individual cell suspension inside microspheres as microcarriers. Examples for such microcarriers are CYTODEX, CYTOPORE and CYTOLINE from GE Healthcare, and HyQ® Sphere™ from Hyclone. In one embodiment, adherent cells can be grown on microcarriers or microbeads, and cell-coated microcarriers in spin column can be used to conduct the ligand enrichment process disclosed herein. Cells could also be entrapped in microspheres formed from alginate or lipid bilayers or the like (Cell Encapsulation Technology and Therapeutics By Willem M. Kühtreiber, Robert Paul Lanza, William Louis Chick, Published by Birkhäuser, 1999). In one embodiment, the receptor carrier is a cell of known identity or a cell of known tissue identity grown on a microcarrier.

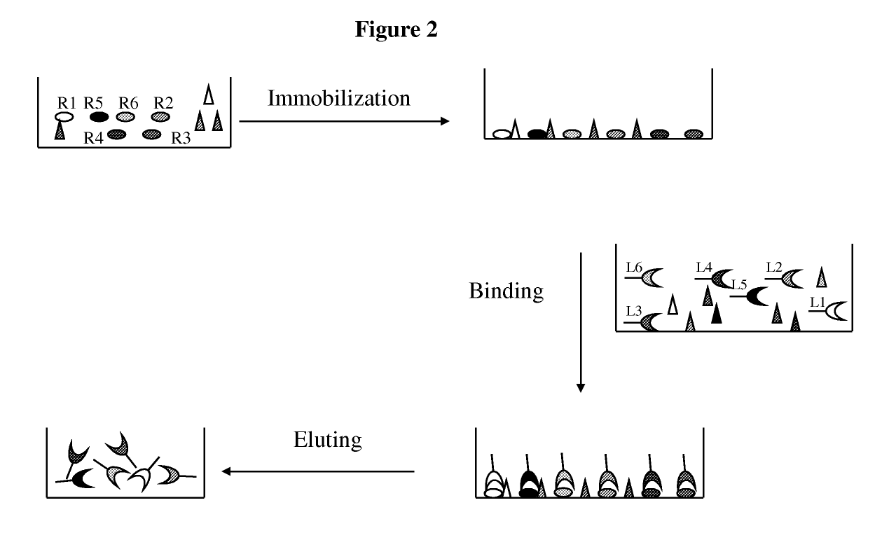

In another embodiment of the invention, each receptor carrier is a substance or an object having a surface, at least part of which is immobilized with a plurality of receptors capable of binding to suitable ligands present in a biological fluid (FIG. 2). The substance or the object may be made of any material capable of immobilizing proteins, peptides or other receptor molecules. Examples of such material include, but are not limited to, plastics, silicon, nylon, metal, paper, agarose, latex or a combination thereof, or other materials listed under biological surface thereof with functionalized surface to facilitate immobilization of proteins and other receptor molecules. The physical shape of the substance or object may be a membrane, a bead, a fiber, a rod, a matrix, a porous structure, a particle, a chip, a well, a vial or a similar container, or the like. Water-soluble proteins such as those present in the cytoplasm generally exert their functionality via interaction with their respective binding partners. To isolate the binding partners of the water-soluble proteins, the water-soluble proteins may be immobilized or embedded onto a suitable substance or object to form a receptor carrier of the present invention. The immobilization process should generally not be too harsh to change the conformations of the receptors. On the other hand, the receptor immobilization should be tight enough so that the receptors stay immobilized when unrelated molecules can be washed away before bound ligands are eluted off with a ligand elution buffer. The association between the immobilized receptors and the substance or object material underneath may be due to non-covalent interaction, covalent bonding, or a combination thereof. Methods of protein immobilization that preserve protein functionalities are well known. Examples of such methods include covalent attachment of proteins and immobilization of biotinylated protein onto streptavidin-coated surfaces (Ruiz-Taylor et al., PNAS 2001, 98:852-857); covalent attachment of proteins to a surface functionalized with amine-reactive groups (MacBeath et al., Science 2000, 289:1760-1763; Zhu et al., Nat Genet. 2000, 26:283-289; Arenkov et al., Anal Biochem 2000, 278:123-131); and covalent immobilization of oxidized glycoproteins onto surface functionalized with aldehyde-reactive groups. Methods are also known to engineer the surface of a substance or object so that non-specific adsorption of ligand molecules can be minimized or avoided (Prime et al., Science 1991, 252:1164-1167) while preserving the desired ligand immobilization. Additional examples of covalently or non-covalently immobilizing proteins onto a surface can be found in the following references: Kenausis et al., J Phys Chem B 2000, 104:3298-3309; MacBeath, G. et al., J. Am. Chem. Soc. 1999, 121:7967-7968; Hergenrother et al., J. Am. Chem. Soc. 2000, 122:7849-7850; Falsey et al., Bioconjugate Chem. 2001, 12:346-353; Houseman et al., Nat. Biotechnol. 2002, 20:270-274; Wang et al., Nat. Biotechnol. 2002, 20:275-280; and Sun et al., Bioconjugate Chem. 2006, 17:52-57.

In one embodiment, the immobilized receptors on the biological surface of the receptor carrier are comprised of extracellular proteins or extracellular domains of receptors. Extracellular domains of most receptors are responsible for ligand binding and are usually soluble in aqueous solution. The immobilization can be through covalent or non-covalent binding. The extracellular proteins and extracellular domains of receptors can be prepared by cleavage of proteins on cell surface by proteases such as trypsin and any other enzymes suitable for releasing them from cell surface. For example, protease TACE has shown to act as a sheddase which specifically release extracellular domain of TNFα from cellular membrane. One of ordinary skill in the art would readily select one or multiple suitable proteases or other enzymes for the cleavage. Various proteases are described elsewhere such as Barrett et al., Handbook of Proteolytic Enzyme 2nd Edn (Academic Press, San Diego, 2004) and Puente et al., Nature Genetics 4:544-558, 2003. Preferably, the extracellular domains of receptors are covalently bound to its biological surface. In such way, a variety of elution conditions can be used to ensure dissociation of ligand molecules from extracellular domains of receptors to which it bound and complete recovery of ligand molecules during the elution step of the invention.

The receptor carrier composed of extracellular proteins and/or extracellular domains of receptors can be prepared from one cell line or multiple cell lines, or from one type of cells or multiple types of cells. The pooled receptor carriers containing extracellular proteins and extracellular domains of receptors from multiple types of cells give a much broader coverage of receptors and are therefore capable of enriching a much broader range of ligands.

In another embodiment, the immobilized receptors on the biological surface of the receptor carrier are comprised of extracellular domains of conventional or nature receptors (ECD) and extracellular proteins. Each of ECD and extracellular proteins that are intended to be immobilized onto the biological surface to generate artificial receptor carriers (collectively called “receptor” in this application) can be prepared by conventional recombinant protein technologies, pooled selectively and immobilized on the biological surface of the receptor carrier. For example, each receptor is prepared as immunoglobulin Fc portion (Fc) fusion protein for easy purification and immobilization on the biological surface. The receptor/Fc fusion gene can be constructed by fusing Fc portion of Ig gene to the C-terminal of the receptor gene. Various Fc-fusion construction vectors with or without signal sequence for Fc-fusion protein secretion are commercially available from companies such as Invitrogen (San Diego, Calif.). The fusion gene can then be introduced into a variety of mammalian cells such as CHO, COS, HEK293 and hybridoma cells. The receptor/Fc fusion proteins can then be collected from the supernatant of the cells and purified by protein A or protein G column. The purified receptor/Fc fusion proteins can be pooled and immobilized on a biological surface to construct a receptor carrier. The biological surface can be coated with protein A or protein G for direct immobilization of receptor/Fc fusion proteins. The immobilization can be further strengthened by using a crosslinker such as disuccinimidyl suberate (Pierce, Rockford, Ill.) to covalently link the receptor/Fc fusion protein to the biological surface. The methods of preparing, producing and purifying Fc fusion protein have described elsewhere (Kurschner et al., 1992, J. Biol. Chem. 267:9354; Bennett et al., 1991, J. Biol. Chem. 266:23060). In addition, receptor proteins can also be prepared through other recombinant protein preparation methods such as methods described in Current Protocols in Protein Sciences, 2007, by John Wiley and Sons, Inc.

In another embodiment, the receptor carrier is comprised of soluble secreted proteins, or conventionally called ligand polypeptides. Soluble secreted proteins include growth factors, cytokines and chemokines (see Appendix B). Extracellular domains of certain receptors are released from cellular membrane by cellular sheddases into biological fluids. Additionally, extracellular domains of receptors can also be released into biological fluids by various other natural physiological, pathological or biological events such as apoptosis, necrosis, tumor growth and metastasis. The extracellular domains of receptors in biological fluids can bind to their corresponding ligand and therefore can be enriched by receptor carrier containing the corresponding immobilized ligands. This type of receptor carrier can be used to enrich extracellular domains of receptors shed from cancer cells into serum or other biological fluids for early diagnostic detection. The secreted proteins for immobilization can be naturally occurring or produced by recombinant techniques, full-length or partial ligand polypeptides that are capable of binding to their receptors or mixture thereof. For example, one approach to prepare cell-wide soluble secreted proteins is to isolate polyA RNA from membrane bound ribosomes of a target cell where secreted proteins are translated. The cellular secreted proteins can then be obtained by in vitro translation using the obtained polyA RNA enriched for secreted proteins. Such methods have been described by Diehn et al., Nature Genetics 2000, 25:58-62. The secreted proteins could be a ligand polypeptide or a receptor. However, receptor molecules tend not to be soluble in aqueous solution. By obtaining soluble secreted proteins after in vitro translation, ligand polypeptides from the targeted cells are obtained for further immobilization onto a biological surface to construct a receptor carrier. Ligand polypeptides can also be prepared individually as recombinant proteins with or without a tag and then immobilized onto a biological surface to generate an artificial receptor carrier.

In still another embodiment of the invention, the receptor carriers may be imbedded in a matrix such as a porous material, e.g. a hollow fiber, a gel or tissue, wherein the imbedded receptor carriers are capable of interacting with the respective ligands that may be present in a suitable sample. In one embodiment, the embedded receptor carriers are cells within a biological tissue matrix, wherein the tissue may be fixed or unfixed. Tissue or cells can be fixed by a number of agents and methods currently known in the art (e.g. formaldehyde fixation). It is understood that in order for the tissue to be suitable for the purpose of the present invention it may need to be treated to remove or immobilize any extracellular free-flowing proteins or extracellular proteins loosely associated with the cell membranes or tissue matrix so that these proteins will not interfere with subsequent ligand enrichment process. The pretreatment may involve extensive washing of the tissue with a suitable buffer, or fixation of the tissue with a suitable fixation agent. If the tissue is an organ, perfusion can be used for washing, delivering ligand molecules and elution.

A suitable sample for the present invention is generally a homogeneous solution comprising or thought to comprise of ligands. The ligands can be in native form or chemically modified form such as biotinylated, or labeled with a stable isotope, a radioactive isotope, or a fluorescence dye and so on. A biological sample may be directly suitable if it is homogeneous and is of proper concentration. A biological sample may also need to be pretreated before it is suitable. Typical sample pretreatments may include homogenization of the sample, removal of any insoluble materials from the sample via known methods such as filtration, centrifugation or the likes, and/or proper dilution or concentration via known methods. For example, a tissue sample may be homogenized, membrane-filtered or centrifuged to remove any insoluble substances and properly diluted to yield a suitable sample; and a blood sample may be centrifuged to remove the blood cells, followed by proper dilution to result in a suitable sample. Typical examples of biological samples include body fluids such as blood, blood plasma, blood serum, hemolysate, spinal fluid, urine, lymph, synovial fluid, saliva, sperm, amniotic fluid, lacrimal fluid, cyst fluid, sweat gland secretion and bile. Additional examples of biological samples include tissue, culture cells, bacteria and viruses as well as medium supernatants and lysates obtained from a specific part of or whole cells, bacteria or viruses.

Once a suitable sample is prepared, it is incubated in an appropriate vessel with a plurality of receptor carriers for a time sufficient for the ligands present in the sample to bind to the receptors on the receptor carriers. In one embodiment, the incubation time is from about 10 minutes to about 2 hours. The incubation temperature is preferably from around 4° C. to around 37° C. Optionally, to minimize non-specific binding of non-ligand proteins in the sample to the surface of the receptor carriers, a blocking solution containing a suitable amount such as 1-10 mg/mL of BSA or IgG or other known proteins is incubated with the receptor carriers for from about 30 minutes to about 2 hours at a temperature from about 4 to about 37° C. The blocking solution is then removed from the receptor carriers. Although BSA, IgG may be introduced as an additional irrelevant protein, it is easily distinguishable from the other ligands in downstream analysis because of its known identity and known physical and biochemical properties and can be removed by their complementary molecules such as anti-BSA, or anti-IgG. Labeling of ligand molecules in the biological sample before subjecting to enrichment by receptor carriers will eliminate interference in downstream analysis by the blocking proteins such as BSA and IgG. The receptor carriers are then incubated with a suitable sample as described above.

Once the ligands are fully bound to the receptors, the remaining sample is separated from the receptor carriers using any of the known procedures used for separating a liquid from a solid or a semi-solid. Examples of such methods include centrifugation of the solid-liquid mixture and aspiration of the liquid phase using a vacuum device. Optionally and preferably, the separated receptor carriers are further washed one or more times with PBS buffer or another solution that does not disrupt ligand/receptor binding to remove any residual non-ligand proteins or other entities that may be associated with the receptor carriers.

Alternatively, the receptor carrier can be separated from the liquid by filtration. The filtration can be achieved through applying vacuum to remove liquid from the receptor carrier. Filtration can also achieved through centrifugation with spin columns to remove liquid from the receptor carrier. In one embodiment, spin column is used to separate receptor carriers from liquid by centrifugation. Examples of such spin columns include Microsep Centrifugal Devices from Pall Life Sciences (East Hills, N.Y.), MWCO Devices from VWR (West Chester, Pa.) and Vivaclear Mini Clearifying Filter from Vivascience (Stonehouse, UK).

In one embodiment of the present invention, the receptor carriers are cells such as live cells. Live cells are expected to have a full range of functional receptors on their surfaces and are thus more likely to capture most of the biologically relevant ligands present in a suitable sample. Various methods can be used to maximize the ligand-binding capacity of live-cell receptor carriers. One method is to starve the cells before incubating with a suitable sample to avoid inaccessibility of receptors to bind to ligands in a suitable sample due to occupancy of similar ligands present in serum used for cell culture. For example, cells can be starved about one hour to about overnight in a serum-free medium or low serum medium before incubating with a suitable sample. Preferably, prior to mixing the receptor carriers and the suitable sample, any culture medium for the cells is removed and the cells are washed using a suitable method. For adherent cells, the culture medium may be removed by aspiration. For cells in a suspension, the culture medium may be removed by centrifugation.

In another embodiment, each receptor carrier is a cell treated with exocytosis inhibitors and/or endocytosis inhibitors. Exocytosis inhibitors are used to block or minimize secretion of cellular proteins from cells into ligand containing samples. Endocytosis inhibitors are used to block or minimize endocytosis of receptors to maximize ligand recovery. Some endocytosis inhibitors can also inhibit exocytosis. Examples of inhibitors for exocytosis or/and endocytosis include, but are not limited to, peroxide, nitric oxide, N-ethylmaleimide, EDTA, thiolate (ABD Bioquest, Sunnyvale, Calif.), NSF peptide (Matsushita et al. 2005, Molecular Pharmacology 67:1137, J Pharmacol and Exp Therapeutics, 314:155, 2005), vaculin-1, cyclosporin A, stilbene analogs such as suramin (PNAS 86:5839-5843, 1989), wortmanin, polylysine, diethylcarbamazine, phenothiazine, 3-amino-triazole, simvastatin, trifluoperazine, carbonyl cyanide p-trifluoromethoxyphenyhydrazone, neomycin, amiloride, GTPγS, phenylarsine oxide, rapamycin, phalloidin, jasplakinolide, quinolines, artemisinin, ethanol, ammonium chloride, trifluorperizine, caImidozolium, penfluridol, pimozide, promethazine, chlorpromazine, imipramine, okadaic acid, methyl-beta-cyclodextrin, chymotrypsin substrate analogs (J Cell Biology, 103:1807-1816, 1986), sucrose, nordihydroguairetic acid, chloroquine, monensin, vinblastine, methylamine, benzyl alcohol, cytochalasin B, oligomycin, dansylcadaverine, amantadine, and rimantadine, Concanavalin A, acetic acid, putrescine, mondansylcaderine, cytochalasin B, aluminum fluoride, nocodazole, chlorpromazine, methyl-5-cyclodextrin, nystatin, long chain amines, brefeldin A, exo 1, colchicine, filipin, chlorpromazine, mondansylcadaverine, statins, methyl-b-cyclodextrin, nystatin, cytochalasin D, latrunculins, Ly290042, nitrocarboxyphenyl-N, N-diphenylcarbamate, U73122 and the like. More examples of exocytosis and endocytosis inhibitors can be found in Current Topics in Membranes and Transport, Vol 32, 1988.

In another embodiment, the receptor carrier has been pre-treated to minimize shedding of non-sample derived ligands from the receptor carrier. For example, the receptor carriers are fixed cells having been pre-treated to remove cellular proteins that can be eluted together with enriched ligands during the elution step. Cells can be fixed by a number of agents and methods currently known in the art (e.g. formaldehyde fixation). The pre-treatment can be stripping cellular proteins from the fixed cells with the elution buffer to get rid of cellular proteins loosely associated with cell membrane before contacting the fixed cells with samples. Various elution buffers described herein or known in the art can be used for pre-treatment. The elution buffer for pre-treatment can be the same or different from the elution buffer used later on for eluting enriched ligands from the fixed cells. The stripped cells can then be neutralized to the physiological condition and used to enrich ligands from samples.

Incubation of receptor carriers such as live cells with a suitable sample is preferably carried out at a lower temperature such as around 4° C. in order to minimize any receptor internalization (PNAS 89:2854-2858, 1992; Am. J. Physiol 129, F46-F52). Typical incubation time at 4° C. is from about 10 minutes to about 2 hours. Following the incubation, cells associated with the ligands are separated from the remaining sample using either centrifugation (for cells in suspension) or aspiration of the liquid phase (for adherent cells). Optionally and preferably, the separated cells are further washed one or more times with a suitable buffer with a near physiological pH such as a PBS buffer to remove any residual non-ligand proteins that may be associated with the receptor carriers.

The ligands associated with the receptor carriers are next dissociated from the receptors by incubating the ligand-bound receptor carriers in a ligand elution solution at appropriate temperature such as from about 4° C. to about 37° C. for a sufficient amount of time such as from about 5 minutes to about 30 minutes. The ligands and receptors are usually bound by physical interactions such as hydrophobic interaction (Van der Waals interaction), hydrogen bonding, electrostatic interaction, or a combination thereof. These forces are typically strongest when the receptor-ligand complex is in an aqueous buffer with physiological pH and ionic strength. Thus, any deviation in pH or ionic strength or both pH and ionic strength from their physiological states will weaken the ligand-receptor interaction. In addition, certain agents such as so-called chaotrope agents are commonly used to weaken physical interactions between ligand and receptor. The exact choice of a suitable elution solution may depend on the nature of the interaction between the ligand and receptor.

In general, a suitable elution solution for the invention is one that is capable of weakening the ligand-receptor interaction without chemically damaging the structure of the ligand. A suitable elution solution should also preferably not extract the receptors off the receptor carriers. Typically, a suitable elution solution may be a buffer having a pH substantially different from the physiological pH such as a pH of 2.5-3 or a pH of 9.5-11.5. For example, a suitable elution solution is a pH 2.5-3 or pH 9.5-11 buffer comprising a chaotrope agent. When the receptor carrier is a cell, an additional salt such as NaCl at around 150 mM is also a component of the elution solution to maintain the cell in an isotonic state. One of ordinary skill in the art would readily derive a suitable elution solution. An example of elution solution for cell-based receptor carriers is a pH 2.5-3.0 buffer comprising 50-100 mM glycine and 150 mM NaCl. This buffer effectively dissociates most protein-protein binding interactions without permanently affecting protein structure. Table 1 lists examples of ligand elution solutions. Some of them are suitable for eluting ligands from live cells as the receptor carrier.

TABLE 1
List of Elution Solutions For Ligand Dissociation

| | Elution Solution |
|---|---|
| | (150 mM NaCl is added for elution solution for |
| | cell-based receptor carriers to maintain |
| Elution Condition | isotonic condition of the cells) |
| Low pH | 100 mM glycine HCl, pH 2.5-3.0 |
| | 100 mM citic acid, pH 3.0 |
| High pH | 50-100 mM triethylamine or triethanolamine, pH 11.5 |
| | 150 mM ammonium hydroxide, pH 10.5 |
| | 0.1 M glycine NaOH, pH 10.0 |
| Ionic strength | 5 M lithium chloride |
| | 3.5 M magnesium or potassium chloride |
| | 3.0 M potassium chloride |
| | 2.5 M sodium or potassium iodide |
| | 0.2-3.0 M sodium thiocyanate |
| | 0.1 M Tris-acetate with 2.0 M NaCl, pH 7.7 |
| Chaotropic effect | 2-6 M guanidine HCl |
| | 2-8 M urea |
| | 1.0 M ammonium thiocyanate |
| | 1% sodium deoxycholate |
| | 1% SDS |
| | 10% dioxane |
| | 50% ethylene glycol, pH 8-11.5 |
| Low pH & Ionic | 50 mM glycine HCl, pH 2.5-3.0, 0.5M NaCl |
| strength | |

Following ligand dissociation from the receptors, the elution solution containing the eluted ligands is separated from receptor carriers using a suitable means such as centrifugation, pipetting, aspiration or the like. If the elution solution used is either acidic or alkaline, the separated elution solution comprising the ligands may need to be immediately brought to neutrality to avoid ligand degradation using either a concentrated alkaline solution or a concentrated acidic solution. For example, if a pH 2.5-3 elution solution is used in ligand dissociation, a 1 M pH 8.5 Tris or Hepes buffer may be used to neutralize the eluted ligand solution. On the other hand, if an elution solution comprising a high salt concentration is used, the eluted ligand solution is usually desalted via dialysis, for example, to avoid protein precipitation. The isolated elution solution may be concentrated to a smaller volume, if necessary, using any of the suitable known concentration methods such as membrane filtration, evaporation using a Speed-Vac and lyophilization, or protein precipitation, etc.

A receptor carrier such as a cell or an organelle on which receptors are not covalently linked to the carriers may shed receptor molecules or other protein, peptide molecules or non-protein/peptide molecules from the receptor carrier during the elution step. The shed molecules from receptor carriers therefore introduce unwanted foreign molecules into the eluted sample in which enriched ligand molecules are present. To differentiate molecules enriched from the biological sample from foreign molecules shed from a receptor carrier or introduced by blocking step, one approach is to pre-label all molecules in the biological sample including ligand molecules with a tag molecule before subjecting them to receptor carrier binding for ligand enrichment. After enrichment of ligand molecules by this invention, the recovered ligand molecules can be separated by various separating methods including proteomics methods and analytical chemistry methods described above and specifically identified by detecting the presence of the tag. The foreign molecules that shed from the receptor carrier or from blocking solution lack the presence of tag and therefore will be undetectable.

In one embodiment, a method of profiling and detecting tagged enriched ligand molecules is to separate them first through one-dimensional or two dimensional electrophoresis followed by transferring ligand molecules onto a matrix such as nitrocellulose paper. The ligand molecules are then detected directly or by a complementary molecule to the tag molecule.

The tag molecule can be any molecule that can be detected directly or indirectly by its complementary molecule. Preferably, the tag is a small molecule whose addition to ligand molecules would not interfere with their binding to receptor molecules on receptor carriers. Examples of tag molecules for direct detection include, but not limited to, fluorescent probes such as fluorescein, Alexa fluor dyes, Cy dyes and many others described in Handbook of Fluorescent Probes, Ninth edition by Richard P. Haugland or elsewhere. Examples of tag molecules detected indirectly by its complementary molecules include, but are not limited to, biotin, fluorescein, or digoxigenin or other haptens described in Handbook of Fluorescent Probes, Ninth edition by Richard P. Haugland or elsewhere. Biotin can be detected by its commercially available complementary molecule avidin, strepavidin, CaptAvidin and NeutrAvidin. Fluorescein and digioxigenin can be detected by its commercially available complementary antibodies specific to each of them. Ligand labeling methods for various tags such as biotin and fluorescence dyes are described in Handbook of Fluorescent Probes, Ninth edition by Richard P. Haugland, or are provided by vendor such as Molecular Probes (Eugene, Oreg.) and Pierce (Rockford, Ill.).

Detection of tag molecules can be achieved through directly linking a detection molecule with complementary molecules. The detection molecule can be a fluorescent molecule or an enzyme that is capable of depositing substrates such as chromogenic substrates, chemiluminescent or fluorescent substrates. The detection molecules and substrates are described in Handbook of Fluorescent Probes, Ninth edition by Richard P. Haugland or elsewhere. Examples are avidin/strepavidin-linked Cy3 (or Cy5), avidin/strepavidin-linked horseradish peroxidase (HRP), avidin/strepavidin-linked alkaline phosphatase (AP), anti-FITC antibody-linked Cy3 (or Cy5), anti-Digioxigenin antibody linked HRP (or AP). Complementary molecules can also be linked to detection molecules indirectly through molecules such as biotin or other haptens such as fluorescein and digoxigenin etc. for amplification. Enzymes-linked biotin or enzyme linked-antibody against the hapten is then used for the detection of complementary molecule such as avidion/strepavidin, avidin-hapten chimera etc. To achieve a greater amplification, multiple layers of biotin and avidin/strepavidin or other haptens such as fluorescein and digoxigenin and their antibodies can be constructed, followed by detection with enzymes-linked avidin/strepavidin or enzyme-linked biotin, or enzyme linked-antibody against the hapten or enzyme linked-hapten.

Detection then proceeds from either fluorescence molecules as a substrate, chromogenic molecules as a substrate, or chemiluminescent molecules as a substrate for the enzyme. See Ausabel et al., eds., in the Current Protocol of Molecular Biology series of laboratory technique manuals. 1987-1997 Current-Protocols, 1994-1997 John Wiley and Sons, Inc. To date, many commercial vendors such as KPL (Gaithersburg, Md.), Pierce (Rockford, Ill.) and Amesco (Solon, Ohio) offer substrates for HRP and AP that allow detection of sub-picogram and even fentogram level of target molecules. Considering the concentration of the least abundance proteins present in serum or plasma is a few picogram per milliliter, 100 uL of serum or plasma sample will provide sufficient amount for even the least abundance proteins to be detected by the current commercial substrates. However, the presence of high abundance proteins still masks the detection of low abundance proteins. Elimination of high abundance proteins by the present invention greatly increases the chance of detecting low abundance proteins since the variety of ligand molecules is much smaller than the proteome and they mostly belong to low abundance proteins.

In another embodiment, the ligands are multiple antibodies each against an epitope of a specific receptor on receptor carriers and are each labeled with a unique identification tag. These tagged antibodies can be used to profile receptors on the receptor carriers. By supplying excess amount of tagged antibodies compared to the amount of their corresponding receptors on receptor carriers, the amount of tags recovered from antibodies bound to receptor carriers is proportional to the amount of the corresponding receptors present on the receptor carriers. Therefore, tagged antibodies can be used to profile expression of receptors on receptor carriers in term of variety and quantities. If the receptor carrier is a cell, tagged antibodies can be used to profile expression of receptors on the surface of the cell. Various types of tags can be used for labeling antibodies. Examples of tags include fluorescence dyes and DNA sequences. If using fluorescence dyes as tags, each unique tag can be a fluorescence dye with a unique excitation or emission wavelength. Preferably, each unique tag has its unique emission wavelength. The fluorescence dyes can be organic dye such as FITC, Cy dyes (GE Healthcare, Piscataway, N.J.), Alexa dyes (Invitrogen, San Diego, Calif.), etc., or inorganic dye such as Qdot nanocrystals (Invitrogen, San Diego, Calif.). If using DNA sequences as tags, each unique tag can be a specific DNA sequence. After recovery of DNA-tagged antibodies which bound to receptor carriers, DNA tags can be cleaved or uncleaved from the antibodies before subjecting to nucleic acid quantification. Various nucleic acid quantification methods can be used. One method of nucleic acid quantification is quantitative polymerase chain reaction (QPCR) that combines nucleic acid amplification and quantification together. Another method is to use polymerase chain reaction for amplification first and then use other nucleic acid quantification methods such as DNA array method to quantify the DNA tags. Various methods for quantifying DNA tags described in patent application publication WO/2003/031591 by L1 Shen et al can be used. If applying to a population of cells, such tagged antibodies can generate receptor expression profile for the population of cells. If applying to a single cell, such tagged antibodies can generate receptor profile of a single cell.

The enriched ligand sample may be suitable for a variety of purposes. One such purpose is the isolation of a ligand of particular interest. In this case, the enriched ligand sample serves as a preliminary purification step. Another purpose is to use the enriched ligand sample for profiling ligands that are present in the original sample prior to the ligand enrichment process and are relevant to the selected biological functionality of interest. Protein profiling yields “finger-print” information on a protein mixture in terms of abundance, integrity, and modification status of the collection of proteins in the mixture. The techniques used for protein profiling are commonly based upon physical and biochemical characteristics of the proteins. These physical or biochemical characteristics include, but are not limited to, molecular weight, isoelectric point (pI), and hydrophobicity/hydrophilicity of the proteins.

Profiling of the enriched ligand sample may be conducted by any or a combination of the Analytical Methods or Proteomic Methods described earlier. If the ligand of interest is of protein or peptide nature, the preferred profiling method is one or a combination of the Proteomic Methods described earlier, for example, 1-D or 2-D gel electrophoresis, chromatography or other means to separate the ligands by molecular weight, pI, hydrophobicity/hydrophilicity, and/or the likes as described in Current Protocols in Protein Science, 2005 by John Wiley & Sons. In one embodiment, the profiling is carried out using 2-D gel electrophoresis coupled with mass spectrometry (MS) and 1-D or 2-D gel electrophoresis coupled with western blotting (see example 6). Other suitable profiling methods include Surface-Enhanced Laser Desorption/Ionization Time-of-Flight MS (SELDI-TOF MS), Liquid Chromatography/MS (LC/MS) and Capillary Electrophoresis (CE)-MS as described by Lambert J. et al., Anal. Chem. 2005, 77:3771-3788.

Alternatively, differences in ligand species between two samples or among multiple samples can be identified using two-dimensional differential in gel electrophoresis (2-D DIGE). In this method, each enriched ligand sample is first minimally and covalently labeled with a unique tag, preferably a fluorescent tag with a unique emission or excitation wavelength. The labeled ligands from two or more samples are then mixed together and subject to separation by 2-D DIGE. Protein spots with differential fluorescent signals are identified, cut out, digested and finally analyzed for their identities via mass spectrometry (Van den Bergh G, Arckens L. 2004. Curr Opin Biotechnol. 15(1):38-43; Baker et al., 2005. Proteomics. 5(4):1003-12; Friedman et al., Proteomics. 4(3):793-811; Zhou et al., 2002, Mol Cell Proteomics. 1(2):117-24). If the sensitivity for direct detection of the fluorescent tag is not sufficient, signal amplification system of the tag may be implemented as described above. However different tags each labeling a sample are needed if enriched ligands from different samples are mixed and separated together. These tags are then detected by their corresponding complementary molecules each labeled with a distinct detection molecule. The amplification system can also be rolling-circle amplification system (Zhou et. al., Genome Biol. 2004, 5(4):R28). For analyzing polypeptide ligands on 2D gel using an amplification system, it is preferred to transfer polypeptides from 2D gel to nitrocellulose/nylon membrane and detect tagged polypeptides by the method of western blotting.

As can be readily appreciated by one skilled in the art, enriched ligand samples for profiling purposes are preferably obtained using an excess of receptors or receptor carries so that enrichment of certain ligands in the samples is not limited by the number of the receptors available. The amount of receptors can be made to be in excess by artificially expressing large quantities of receptors by transfection of expression vectors into the cells as described earlier. The amount of receptor carriers can be made in excess by using a high amount of the receptor carriers. The amount of receptor carriers necessary for achieving “excess state” can be determined by enriching a suitable sample using different amounts of the receptor carriers, followed by profiling of the enriched samples. When the ligand profile of the sample becomes independent of the amount of receptor carriers used, the amount of receptor carriers used is in excess.

Alternatively, a relatively dilute sample may be used to ensure that the receptors or receptor carriers are in excess. For example, a sample with large quantities of ligands can be diluted in a series of 2-fold dilutions. Each step of the dilution will be tested, and the dilution factor necessary for achieving “excess state” of the receptors can be determined by enriching different dilutions of the sample with a fixed number of receptor carriers, followed by profiling of the enriched samples. When the ligand profile of the sample becomes proportional to the dilution factors used, the amount of the receptor carriers is in excess.

Ligand profiling using an enriched ligand sample according to the present invention may have many practical applications. It can be used to map out ligand proteome for any organisms in a given physiological state including, for example, diseased- or nondiseased-state or a particular “emotional” state. By comparing the ligand profiles of enriched ligand samples obtained from the same biological fluid but with different cell-based receptor carriers, one can readily identify any missing receptors that may be relevant to a disease or physiological function.

In one embodiment, ligand profiling according to the present invention may be used to detect pathological conditions that may exhibit a ligand profile characteristic of a particular disease, a diseased state, or discover new disease or diseased state-related biomarkers or new disease targets. This kind of discovery often uses so called “differential profiling” method that is to compare ligand profiles derived from the biological samples from a particular disease or diseased state with the one from its control such as healthy state. Examples of pathological conditions that may be detected with the aid of the present invention include, but are not limited to, diabetes, arthritis, elevated (or reduced) cholesterol levels, cardiovascular diseases such as heart disease and stroke, anemia (for example, sickle cell anemia), cancer, liver diseases (for example, hepatitis), AIDS, kidney diseases, tissue destruction (for example, myocardial infarction), neurodegenerative diseases such as Alzheimer's disease and Parkinson's disease, transmissible spongiform encephalopathy (TSE) such as BSE, autoimmune diseases such as multiple sclerosis (MS), allergies, urticaria, allergic asthma and aging.

In one embodiment, differential ligand profiling can be conducted using serum or plasma from atherosclerosis patients and healthy individuals as biological samples and endothelial cells as the receptor carriers. The resulting differential ligand profiles between atherosclerosis patients and healthy individuals can be used to derive potential new targets for preventing or slowing down atherosclerosis. Differential ligand profiling can also be conducted using smooth muscle cells as the receptor carriers to compare ligand profile among serum samples from normal individual and heart attack patients before and after heart attack to identify new biomarkers for early detection of heart attack.

Differential ligand profiling can also be applied to identify new satiety molecules for obesity prevention and therapy. Considering hypothalamus is the satiety control center, therefore, one approach to discover new satiety molecules is to use hypothalamus cells as the receptor carrier to compare ligand profiles between serum or plasma samples of hungry state and full state of the same individual. The ligands to hypothalamus cells whose amount is increased in the full state compared to hungry state are candidates of satiety molecules. Differential ligand profiling can also be conducted between obese individuals' and normal individuals' serum or plasma samples using hypothalamus cells as the receptor carrier to discover differentially expressed ligands that may serve as new therapeutic means for obesity.

Differential ligand profiling can also be applied to identify early cancer diagnostic markers. Many cancer cells develop an autocrine system to sustain uncontrolled growth. In such autocrine systems, cancer cells secret a growth factor that is ordinarily absent in normal cells and stimulates its receptor on the same cancer cell. Therefore, the newly secreted growth factors may serve as early cancer diagnostic biomarkers. This kind of biomarker can be identified by differential profiling on biological fluids such as sera between cancer patients and normal individuals, or between sera collected before and after cancer surgery of the same patient using his/her cancer cells as receptor carrier.

Differential profiling can also be applied for identifying novel ligands for orphan receptors. This can be achieved by contacting a biological sample suspected to contain a ligand for the desired orphan receptor with two cell populations (one expressing the orphan receptor and another not) separately to derive two separate ligand profiles. The ligand that presents in the ligand profile of orphan receptor-expressing cells, but not in the ligand profile of orphan receptor null cells is the potential ligand for the orphan receptor.

In another embodiment of differential profiling of the invention, a drug screening method is provided, wherein the method comprises the steps of: 1) preparing an enriched reference ligand sample from a biological fluid without the presence of a drug candidate using a receptor carrier according to the present invention; 2) preparing an enriched target ligand sample from a biological fluid with the presence of a drug candidate by using the same receptor carrier as in step 1; 3) determining the profile of the enriched reference ligand sample using a suitable profiling method; 4) determining the profile of the enriched target ligand sample using the same profiling method; and 5) assessing the effectiveness of the drug candidate by comparing the above two profiles.

In yet another embodiment of differential profiling of the invention, a therapeutic evaluation method is provided, wherein the method comprises the steps of: 1) preparing an enriched reference ligand sample from a biological fluid of a patient before therapeutic treatment using a receptor carrier according to the present invention; 2) preparing an enriched treatment ligand sample from the same type of biological fluid of the same patient after therapeutic treatment using the same receptor carrier as in step 1; 3) determining the profile of the enriched reference ligand sample using a suitable profiling method; 4) determining the profile of the enriched treatment ligand sample using the same profiling method; and 5) identifying biomarkers for evaluating the effectiveness of the therapeutic treatment by comparing the above two profiles and correlating each profile with patient treatment result.

In still another embodiment of the invention, a method is provided for profiling the receptors on a target cell by using a biological fluid with a known ligand profile. In this method, a biological fluid is first profiled using a reference cell as receptor carrier to produce a reference ligand profile, which in turn indirectly gives the receptor profile of the reference cell. The same biological fluid is then profiled using the target cell as receptor carrier. The ligand profiles from the reference cell and the target cell are compared. Any missing ligand(s) from the ligand profile generated by the target cell indicates an undetectable amount, or a lack of the corresponding receptor(s) on the target cell. Conversely, any additional ligand(s) from the ligand profile generated by the target cell indicates the presence of new receptors on the target cell. This method may be applied to discover diseases or pathological conditions by comparing the receptor profile of a reference cell such as a healthy cell with the receptor profile of a diseased cell or of a cell associated with a pathological condition.

In one embodiment, the present invention provides a method of ligand profiling of one or more distinct samples each comprising mixtures of ligand molecules, the method comprising: contacting each of the distinct samples with one or more populations of receptor carriers, wherein each receptor carrier comprises a plurality of receptors to which the ligand molecules may bind; washing unbound ligand molecules away and eluting the bound ligand molecules from each population of the receptor carriers to provide separate ligand fractions; and fractionating the ligand fractions to give separate profiles of ligand molecules for each of the distinct samples. The one or more populations of receptor carriers can be same or different from each other. In general, the receptor carriers can be live cells, fixed cells, a mixture of cells, organelles (live or fixed), cell ghost, cellular membranes, vesicles comprising a plurality of receptors, or artificial biological surface comprising a plurality of immobilized receptors. In one embodiment, the cells are treated with inhibitor of exocytosis or inhibitor of endocytosis. In another embodiment, the cells are treated to get rid of cellular proteins loosely associated with cell membrane before contacting the cells with samples.

The present invention also provides a kit for enriching multiple ligands from a sample comprising ligands with unknown identity or quantity, the kit comprising a binding solution, a washing solution, an elution solution and an instruction on experimental procedures according to the methods disclosed herein.

### EXAMPLE 1

### Ligand and Enrichment of a Human Serum Sample Using Hela Cells and NIH3T3 Cells as Receptor Carriers

A confluent monolayer of Hela or NIH3T3 cells in a 10-cm culture plate was first washed with 10 mL DMEM medium without serum and then replenished with 10 mL DMEM medium without serum, followed by incubation in a tissue culture incubator for 1 hour. After the incubation, DMEM medium was removed and the Hela cells were washed again with ice cold PBS, followed by incubation with 2.5 mg/mL IgG or 2-10 mg/mL BSA in ice cold PBS or PBS only at 4° C. for 30 min on a shaker to derive “prepared” Hela or NIH3T3 for ligand enrichment. After the liquid was removed, the prepared cells were incubated with 2 mL of human serum diluted 1:20 or 1:50 in PBS for 30 minutes at 4° C. on a shaker to allow ligand-receptor association. The liquid was then removed from the cells bound with the ligands by aspiration. The ligand-bound cells were washed with PBS 1-3 times to remove any residual unbound proteins and nonspecific binding proteins, and then incubated in 1.5 mL elution buffer (50 mM Glycine, pH 3.0 with 150 mM or with 500 mM NaCl) at 4° C. for 10 minutes to dissociate ligands from the cell membrane. The ligand-containing elution buffer was then removed from Hela or NIH3T3 cells, centrifuged to discard residual Hela or NIH3T3 cells, and neutralized to pH 7.5 by HEPES.

### EXAMPLE 2

### EGF Enrichment Using Hela Cells as Receptor Carriers

One hundred μL of human serum with spiked recombinant EGF were diluted into 2 mL (1:20 dilution) with ice cold PBS and added into prepared Hela cells using IgG as blocking agent for ligand enrichment according to the description in Example 1. Two mL ice cold PBS without serum and recombinant EGF was used in parallel as the control. The solution of 500 mM NaCl and 50 mM Glycine pH3.0 was used for ligand elution.

The following samples were obtained during the enrichment process: 1) eluted ligands from the Hela cells incubated with serum and spiked EGF (EnriSerumEGF); 2) eluted solution from Hela cells incubated with PBS (Control); 3) 1:20 dilution of the serum with spiked EGF solution before incubating with Hela cells (SerumEGF) and after 30 min incubation with Hela cells (PostSerumEGF). A 1:10 dilution of serum was used for quantifying concentration of EGF present in the naive serum. One hundred μL of solution from each of the above samples were used to quantify the concentration of EGF present in each sample using Human EGF ELISA Development Kit (PeproTech, N.J.). Total EGF amount in each sample was calculated based on the derived concentration and the total volume of each sample. Total protein concentration in each sample was quantified by Quant-iT Protein Assay Kit (Invitrogen, CA). The amount of IgG present in the eluted ligand sample was estimated by gel electrophoresis followed by protein stain with Lumitein (Biotium, CA). The recovered ligand protein concentration was estimated by subtracting IgG concentration in the eluted ligand sample from its total protein concentration. The estimated ligand protein concentration will be higher than the actual concentration of the eluted serum-derived ligands since the estimated value does not preclude proteins shed from Hela cells during elution. Therefore, the actual enrichment fold should be higher than the value reported in Table 2.

As shown in Table 2, EGF recovery rate by a confluent plate of Hela cell was 73%. The percentage of EGF in the enriched ligand sample was 0.0018% since the total amount of all recovered ligand proteins is estimated to be only 10 ug. Compared to the percentage of EGF in unenriched serum (0.0000048%), EGF has been enriched 375 fold through a single enrichment step by Hela cells.

TABLE 2
EGF Enrichment by Hela cells

| | EGF | | |
|---|---|---|---|
| Samples | (pg) | Recovery Rate | Enrichment Rate |
| Serum | 50 | NA | NA |
| SerumEGF | 240 | NA | NA |
| PostSerumEGF | 60 | NA | NA |
| EnriSerumEGF | 176 | 73% | 375 |
| Control | 0 | NA | NA |

### EXAMPLE 3

### Efficiency of PDGFaa Enrichment is Associated with Abundance of PDGF Receptor Alpha on the Cell Surface

To compare PDGFaa enrichment efficiency between NIH3T3 cells with high expression level of PDGF receptor alpha and Hela cells with low expression level of PDGF receptor alpha, 100 mL of human serum plus 400 pg spiked recombinant PDGFaa were diluted into 2 mL (1:20 dilution) with ice cold PBS and added into prepared Hela and NIH3T3 cells without blocking step for ligand enrichment according to the description in Example 1. Two mL ice cold PBS without serum and recombinant PDGFaa was used in parallel as control. A solution of 150 mM NaCl and 50 mM Glycine pH 3.0 was used for ligand elution. After ligand elution, cell lysates were prepared from Hela cells and NIH3T3 cells to confirm differences in PDGF receptor alpha expression levels.

The following samples were obtained: 1) eluted ligands from the Hela cells incubated with serum and spiked PDGFaa (EnriSHela); 2) eluted ligands from the NIH3T3 cells incubated with serum and spiked PDGFaa (EnriSNIH3T3); 3) control eluted from Hela cells incubated with PBS (ControlHela); 4) control eluted from NIH3T3 cells incubated with PBS (ControlNIH3T3). These samples were concentrated by membrane filtration using a Microsep 10K Omega from Pall Life Sciences (East Hills, N.Y.) to give 100 μL-200 μL concentrated samples. Seventy-five μL of concentrated solution from each of the above samples were used to quantify the amount of PDGF present in each sample using Human/Mouse PDGF-AA Immunoassay Kit (R&D Systems, MN). Seventy-five μL of 1:10 dilution of the serum was used for quantifying concentration of PDGFaa present in the naive serum. The total PDGFaa amount was calculated based on the concentration and the total volume of each sample. The total protein concentration in each sample was quantified by Quant-iT Protein Assay Kit (Invitrogen, CA). This protein concentration value will be higher than the actual concentration of the eluted serum-derived ligands since this value does not preclude proteins shed from Hela or NIH3T3 cells during elution. Therefore, the actual enrichment fold should be higher than the value reported here.

As shown in Table 3, NIH3T3 cells are more efficient at enriching PDGFaa than Hela cells.

TABLE 3
PDGF Enrichment by Hela & NIH3T3

| | PDGF | | Enrichment |
|---|---|---|---|
| Samples | (pg) | Recovery Rate | Rate |
| Serum | 250 | NA | NA |
| SerumPDGF | 650 | NA | NA |
| EnriSHela | 36 | 3% | 6 |
| EnriSNIH3T3 | 80 | 12% | 42 |
| ControlHela | 0 | NA | NA |
| ControlNIH3T3 | 0 | NA | NA |

### EXAMPLE 4

### Increased Salt Concentration Enhances PDGFaa Elution Efficiency from Hela Cells

To optimize elution efficiency of PDGFaa, two elution buffers varying at salt concentration were tested using PDGFaa and Hela cell system shown in Example 3. Low salt elution buffer contains 150 mM NaCl, 50 mM Glycine pH 3.0 while high salt elution buffer contains 500 mM NaCl, 50 mM Glycine pH 3.0.

As shown in Table 4, the high salt elution buffer is more efficient than the low salt elution buffer at eluting PDGFaa from Hela cells.

TABLE 4
Elution efficiency Affected by Salt

| | | | Enrichment |
|---|---|---|---|
| Elution Buffer | Recovered PDGF | Recovery Rate | Rate |
| Low Salt | 68 pg | 10% | 9.6 |
| High Salt | 92 pg | 14% | 16 |

### EXAMPLE 5

### The Relation Between PDGFaa Concentration and Recovery Rate

To study the relationship between ligand concentration used for enrichment and ligand recovery rate, three samples derived from 100 mL of the same serum but with different PDGFaa concentrations or total amount of PDGFaa were used for ligand enrichment using Hela cells according to the procedure described in Example 3. These three samples were: 1) 2 mL of 1:20 dilution of serum containing 125 pg/mL PDGFaa (Sample 1); 2) 2 mL of 1:20 dilution of serum plus spiked PDGFaa containing 325 pg/mL PDGFaa (Sample 2); 3) 5 mL of 1:50 dilution of serum plus spiked PDGFaa containing 130 pg/mL PDGFaa (Sample 3).

As shown in Table 5, PDGFaa recovery efficiency is proportional to the concentration of PDGFaa used for enrichment, and not related to the total amount of PDGFaa. However the total PDGFaa recovered amount is related to both PDGFaa concentration and total amount of PDGF exposed to Hela cells.

TABLE 5
Comparison of PDGFaa Concentration and Recovery Rate

| | PDGF | Total | Recovered | |
|---|---|---|---|---|
| Name | Conc. | PDGF | PDGF | Recovery Rate |
| Sample 1 | 125 pg/mL | 250 pg | 13 pg | 5.2% |
| Sample 2 | 325 pg/mL | 650 pg | 70 pg | 10.7% |
| Sample 3 | 130 pg/mL | 650 pg | 34 pg | 5.2% |

### EXAMPLE 6

### Labeling Serum Polypeptides/Proteins with Biotin Before Enrichment for Specific Detection of Serum-Derived Ligands

One hundred microliters (100 μL) of a human serum was mixed with 300 μL PBS and 100 μL of 0.5M sodium bicarbonate pH 8.5 to derive serum reaction solution with approximately 10 mg/mL protein concentration and pH at 8.5. Eighty microliters (80 μL) of 20 mg/mL biotin-XX-SE (Biotium, Hayward, Calif.) was then added dropwise into this serum reaction solution followed by gentle rocking at room temperature for 1 hour. One hundred microliters (100 μL) of 1.5M L-lysine, pH 8.5 were then added to stop the reaction.

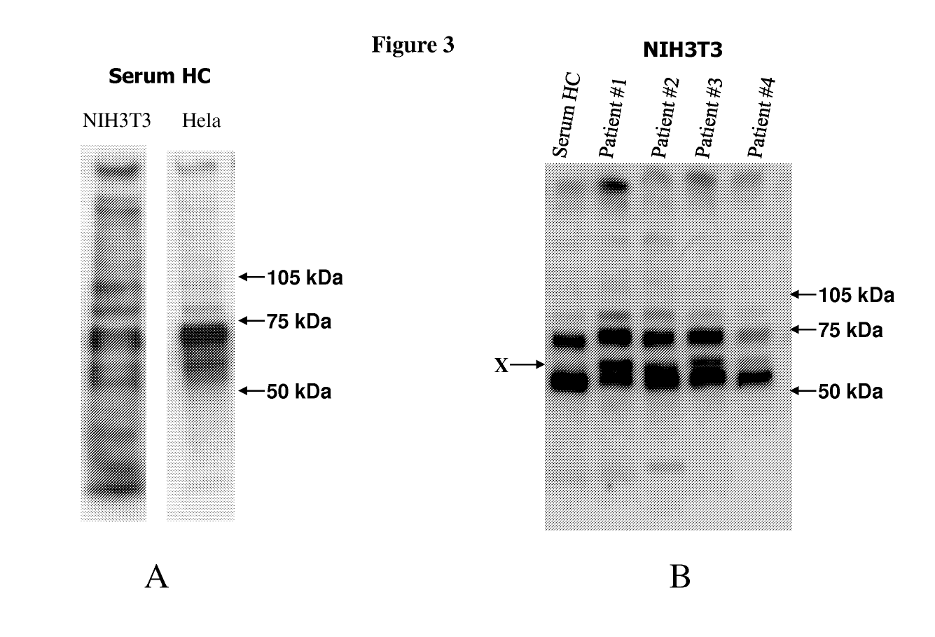

The biotin-labeled serum solution was either neutralized with 120 μL HEPES pH 7-7.5 to adjust pH into pH 7-7.5 before mixing with 2 mL PBS (FIG. 3A) or directly mixed with 2 mL PBS (FIG. 3B) and adding onto prepared Hela or NIH3T3 cells that were blocked with BSA solution. Ligands to Hela or NIH3T3 cells were then derived by following example 1.

Twenty microliters (20 μL) each of ligand samples was subject to 1-D SDS-PAGE electrophoresis using pre-cast 4-15% gradient acrylamide gels from Bio-Rad (Hercules, Calif.) in Tris-HCl buffer. SDS-PAGE were conducted on a Mini-Protean 3 gel electrophoresis system from Bio-Rad using Tris/Glycine buffer (20 mM Glycine, 2.5 mM Tris and 0.1% SDS) as the running buffer and with the constant current set at 35 mA for one and half hours. After electrophoresis, the proteins on the gels were transferred onto nitrocellulose paper through Mini-Protean 3 Western blot transfer system at 350 mA for 2 hours in transfer buffer (20 mM Tris, 150 mM Glycine, 20% Methanol and 0.038% SDS) on ice.

The nitrocellulose paper with transferred proteins (blot) was then blocked with 3% milk in TBST (10 mM Tris pH 8.0, 150 mM NaCl, 0.05% Tween-20) for 1 hour before subjecting to 1 hour incubation with HRP conjugated strepavidin (BioLegend, San Diego, Calif.). After 3-5 times of washing with TBST, the blot was developed using Western Lightening system from Perkin Elmer (Waltham, Mass.) and the chemiluminescence signal was captured by Amersham Hyperfilm™ ECL (Buckinghamshire, UK).

### EXAMPLE 7

### Differential Ligand and Profiling Among Human Serum Samples

Sera of four multiple myeloma patients (Patient #1-4) and one healthy individual (Serum HC) were labeled with biotin and subjected to ligand enrichment as described in example 6 using NIH3T3 cells as receptor carrier. As shown in FIG. 3B, the profiles of biotin-labeled ligands among multiple myeloma patients shared an elevated level of protein migrated at position “X” as compared to the profile of the healthy individual.

### EXAMPLE 8

### Differential Ligand and Profiling of Two Human Plasma Samples Using 2-D Gel Electrophoresis

Ligand samples L #1 and L #2, obtained each from 5 mL of 1:50 human plasma diluent (Sample #1 or Sample #2) and a confluent monolayer of HeLa cells as receptor carriers in a 10-cm culture plate according to Example 1 with 2 mg/mL BSA as blocking agent, were each subject to protein precipitation by trichloroacetic acid (TCA). The protein precipitate from L #1 was resuspended in 10 μL 2-D lysis buffer (30 mM pH 8.8 Tris-HCl, 7 M urea, 2M thio-urea and 4% CHAPS) and then minimally labeled with Cy3 from GE Healthcare (Piscataway, N.J.) according to the CyDye labeling procedure supplied by the reagent manufacturer. The protein precipitate from L #2 was similarly resuspended in 10 μL 2-D lysis buffer but was labeled with Cy5 also from GE Healthcare according to the manufacturer's recommended procedure.

For the isoelectric focusing (IEF) dimension, the total amount of Cy3- and Cy5-labeled samples were mixed in equal volume followed by the addition of 20 μL 2×2-D sample buffer (8M urea, 130 mM DTT, 4% w/v CHAPS and 2% v/v Pharmalyte™ 3-10 for IEF). The resulting mixed protein suspension was further mixed with 120 μL Destreak solution (7 M urea, 2 M thiourea, 4% CHAPS, 1% w/v bromophenol blue, 100 mM Destreak reagent from GE Healthcare (catalogue number:17-6003-19), and 2% Pharmalytes) and 100 μL rehydration buffer (8 M urea, 4% CHAPS, 1% w/v bromophenol blue, 1% Pharmalytes and 2 mg/mL DTT) to a total volume of 260 μL. After thorough mixing, the mixture was spun. The supernatant (250 μL) was loaded into an IPG strip (13 cm, pH 3-10 linear for IEF) from GE Healthcare. IEF was performed for a total of 25000 volt-hours using standard conditions recommended by the instrument manufacturer GE Healthcare.

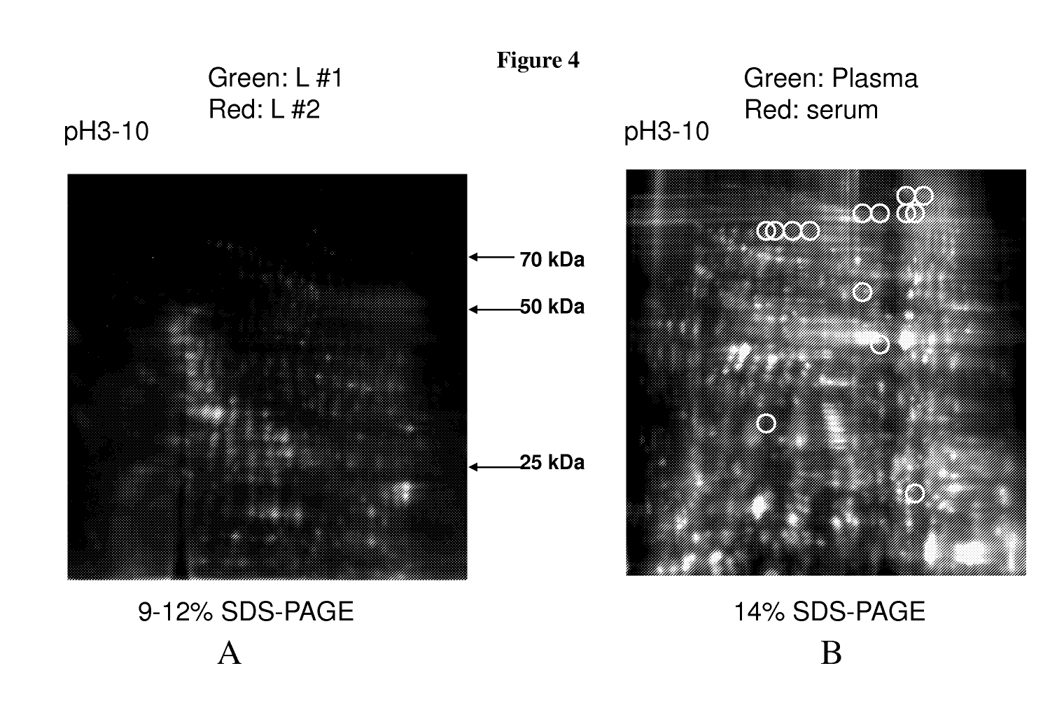

After IEF, the IPG strip was incubated with 10 mL of Equilibration solution 1 (50 mM pH 8.8 Tris-HCl, 6 M urea, 30% v/v glycerol, 2% SDS, 10 mg/mL DTT and 1% w/v bromophenol blue) for 15 minutes with gentle shaking and then with 10 mL of Equilibration solution 2 (50 mM pH 8.8 Tris-HCl, 6 M urea, 30% v/v glycerol, 2% SDS, 45 mg/mL iodoacetamide and 1% w/v bromophenol blue) for 10 minutes with gentle shaking. The IPG strip was rinsed once with SDS gel running buffer (192 mM glycine, 25 mM Tris and 0.1% SDS) and then inserted into a 9-12% gradient SDS gel (18×16 cm, 1-mm thickness). The strip was then covered with 0.5% agarose sealing solution. SDS-PAGE electrophoresis was performed at 16° C. until bromophenol blue reached the bottom of the gel. The result was shown in FIGS. 4, 5 and 6.

After electrophoresis, the 2-D gel was cleaned and immediately scanned using a Typhoon Trio gel scanner by GE Healthcare. Images were analyzed using ImageQuant and DeCyder softwares provided by the gel scanner manufacturer.

As shown in FIG. 4, Hela cells as a receptor carrier effectively enriched a small subset of proteins from human plasma and therefore greatly decreased the complexity of proteins to be analyzed and therefore increased the sensitivity of detecting individual low abundant proteins within the sample. As shown in FIG. 4, most of enriched proteins were low abundant proteins and were not detectable without the enrichment process due to the presence of high abundant proteins in the original human plasma sample. Also as shown in FIG. 4, most of enriched proteins were <50 Kd, demonstrating that the nature of ligand proteins tend to be small in molecular weight.

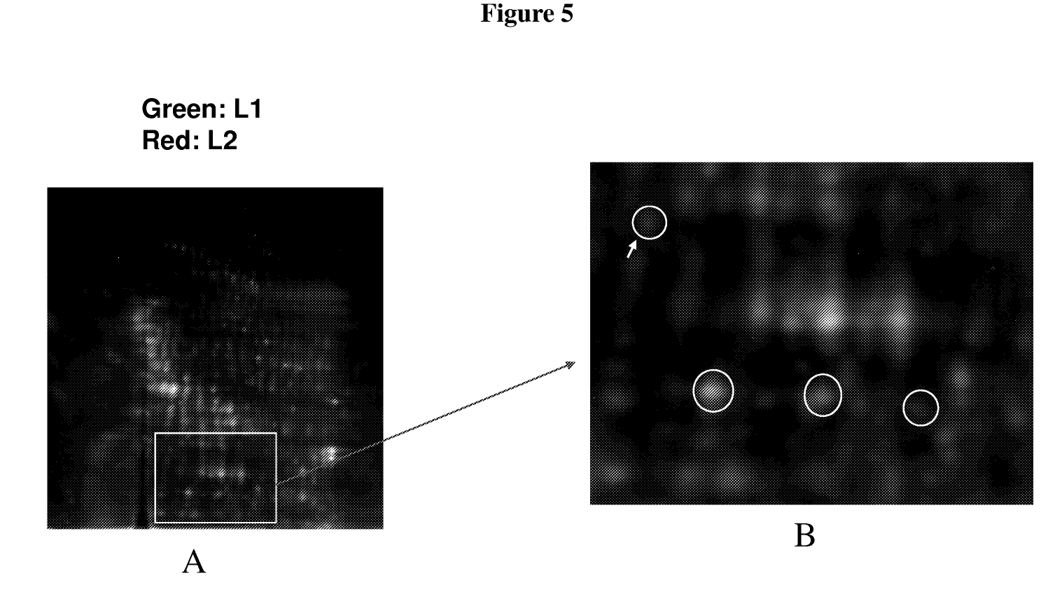

FIG. 5 demonstrated the consistency of this enrichment method since ligand protein profile obtained from different human plasma samples were similar with most ligand proteins present in equal amount, but significant number of ligand proteins vary at their expression level. It demonstrated this enrichment method effectively narrowed biomarker candidates down into a small, very manageable number to be monitored.

### EXAMPLE 9

### Ligand and Enrichment of a Human Plasma Sample Using Three Separate Types of Cells as Receptor Carriers

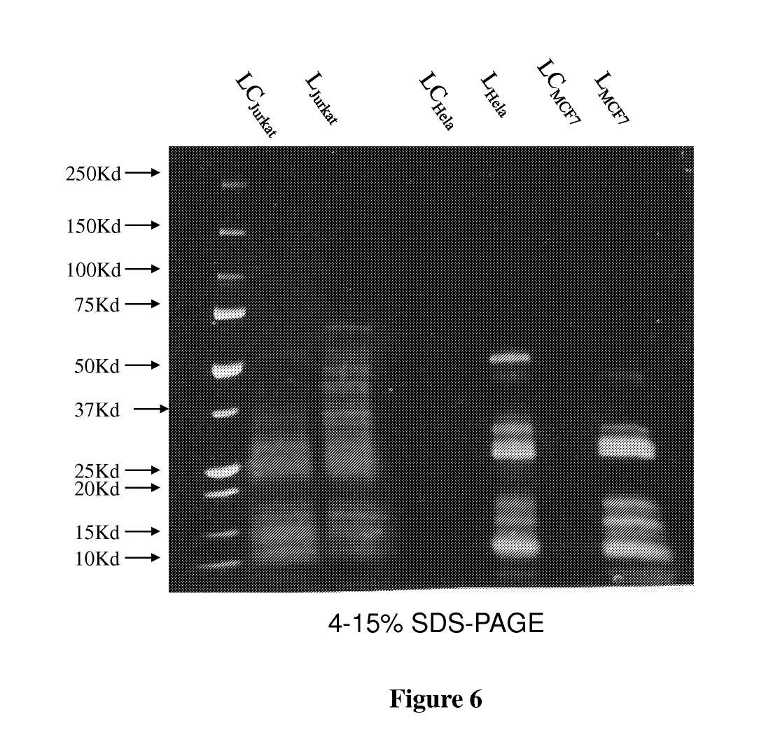

Hela, MCF7 and Jurkat cells were used separately as receptor carriers to enrich a human plasma sample (Plasma 3). 1-D gel analyses of the resulting ligand samples exhibited different ligand profiles among the samples as a result of the differences in the membrane receptor profiles of the three cell lines (FIG. 6). Hela is an epithelial cell line derived from a human cervical adenocarcinoma. MCF7 is an epithelial-like cell line derived from a human breast adenocarcinoma. Jurkat is a human leukemia T cell line.

Ligand enrichment using Hela or MCF7 cells as receptor carriers was carried out according to the procedure used in Example 1 with 2 mg/mL BSA as blocking agent to give a ligand sample L_{Hela} or L_{MCF7}. In order to determine if any of the eluted ligand proteins might be derived from receptor carriers Hela or MCF7 cells, the same cells were also incubated with PBS under the same condition as used for incubation with the plasma sample to result in control sample LC_{Hela} or LC_{MCF7}.

Since Jurkat is a suspension cell line, ligand enrichment using the cell line had to be carried out using a slightly modified procedure. Briefly, about 2.7×10⁷ Jurkat cells were evenly split into two 10 mL-centrifuge tubes (tube #1 and tube #2) and then centrifuged down. The serum-containing RPMI medium in each tube was removed, followed by replenishment with 10 mL RPMI medium without serum. The tubes containing the cells were incubated in a tissue culture incubator for 1 hour. Next, both tubes of Jurkat cells were washed with ice cold PBS once, each followed by 30 minutes of incubation with 5 mL 2 mg/mL BSA in ice cold PBS at 4° C. with shaking. Both tubes of Jurkat cells were spun down again to remove the BSA solution. Jurkat cells in tube #1 were resuspended in 5 mL of 1:50 human plasma 3 diluent in ice cold PBS while Jurkat cells in tube #2 were resuspended in ice cold PBS as a blank control. Both tubes were then incubated at 4° C. for 30 minutes with shaking to allow ligands in the plasma diluent to bind to their respective receptors on the cell membrane or to allow cell membrane-bound proteins, if any, to dissociate into the PBS buffer. The tubes were centrifuged again and the supernatant in each tube was removed. The cells in each tube were next washed with PBS once to remove any residual unbound proteins. To elute the ligands off the cell membranes, 1.5 mL ice-cold elution buffer (50 mM pH 3.0 glycine, and 150 mM NaCl) was added into each tube and the resulting cell suspensions were incubated at 4° C. with shaking for 10 minutes. The ligand-containing elution buffer in tube #1 and the elution solution in tube #2 were then each recovered from Jurkat cells by centrifugation and concentrated to a volume of 50-100 μL using a Microsep 10K Omega (Pall Life Sciences, New York), resulting in a ligand sample L_{Jurkat} and Jukat cell control sample LC_{Jurkat}, respectively.

Ten microliters (10 μL) each of the three ligand samples, L_{Hela}, L_{MCF7} and L_{Jurkat} along with 10 μL each of the three control solutions, LC_{Hela}, LC_{MCF7} and LC_{Jurkat}, was subject to 1-D SDS-PAGE as described in Example 6.

As shown in FIG. 6, ligand samples enriched from the same human plasma sample but with different receptor carriers exhibited different ligand profiles, indicating different membrane receptor profiles for each type of cells. The ligand protein profile of L_{Jurkat} differed significantly from those of L_{Hela} and L_{MCF7} while the ligand protein profiles of L_{Hela} and L_{MCF7} were similar to each other. This may be explained by the similar morphology and functionality of Hela and MCF7 cells and their distant relatedness to Jurkat cells. Since there were no protein bands shown on lanes LC_{Hela} and LC_{MCF7}, it suggested that all proteins shown on L_{Hela} and L_{MCF7} were derived from human plasma while only >30 kD proteins were of human plasma origin as shown on lane LC_{Jurkat}.

### EXAMPLE 10

### Preparation of Human Breast Cancer Cell Line MDA-MB-231 Cells on Microcarriers and Utilization of MD-A-MB-231 Cell Microcarriers as Receptor Carrier

MDA-MB-231 cell line was grown on HyQ® Sphere™ (Hyclone, Logan, Utah) microcarriers with a positive charge to enhance adherence. The microcarriers were prepared according to manufacturer's instructions. Passage 8 of the cell line was grown to 60-70% confluence in Leibowitz-15 media (ATCC, Manassas, Va.) fortified with 10% FBS and antibiotics. The cells were harvested from 10 cm culture plates and inoculated onto microcarriers as per the manufacturer's instructions at a density of 20 million cells for 2 gram of HyQ® Sphere™ microcarrier in 100 mL of media in 125 mL biological spinner flask (Techne, Burlington, N.J.) using intermittent stirring of 2. 5 min stirring at 40 rpm with a 15 minute rest between stirring periods during the entire time in culture. The cells were grown in continuous culture at 37° C. incubator without CO₂ for 6 days with 1 complete change of media during that time. Visual inspection and periodic counting of number of cells/mg microcarrier using trypsin digest technique were used to monitor growth on the microcarriers.

The microcarriers were harvested on the 6^{th} day and washed with PBS once before being re-suspended in ice cold PBS to 66 mg microcarrier/mL. Five hundred microliters were pipetted into an eppendorf tube and centrifuged 2000 rpm for 2 min in an Eppendorf microfuge to remove the PBS. The microcarriers were re-suspended in 0.5 mL of serum cocktail containing spiked FGF and EGF and incubated for 1 hr at 4° C. on a nutator. The microcarriers were washed 3× with 0.5 mL of PBS+5 mM EDTA and centrifuged at 2000 rpm for 2 min between each wash. After the last wash, 0.5 mL of elution buffer was added and the microcarriers were incubated for 10 min at 4° C. on a nutator. After this incubation, the microcarriers were centrifuged at 6000 rpm for 2 min. The microcarriers were discarded and the final eluted sample was neutralized with the addition of 12.5 uL of 0.5 M sodium carbonate, pH 8.5.

The eluted samples were tested as per manufacturer's instructions using R&D Systems (Minneapolis, Minn.) Duo-Set ELISA kits for FGF, EGF and PDGF. The samples were first diluted 1 to 3 in 1% BSA prepared in water before running in the FGF ELISA, but for EGF and PDGF the samples were run undiluted. Table 6 shows the results.

TABLE 6
FGF and VEGF Recovery by MDA-MB-231 Microcarrers

| | Amount Spiked, | Amount Recovered, | |
|---|---|---|---|
| Analyte | pg/mL | pg/mL | % Recovery |
| FGF | 285 | 125 | 44% |
| PDGF | 1242 | 333 | 27% |
| EGF | 121 | 21 | 18% |

### EXAMPLE 11

### Identification of Exocytosis Inhibitors Capable of Blocking Secretion of Growth Factors from Live Cells

Human breast cancer cell line MDA-MB-231 and human fibroblast cell line MRC5 were used to determine if endocytosis and exocytosis inhibitor Brefeldin, Exo1, Phenylarsine oxide, Jasplakinolide and Thiolite can inhibit secretion of FGF-2 and VEGF from MDA-MB-231 and MRC5 cells.

First, MDA-MB-231 and MRC5 cells were each grown on 6-well dish until reaching 95-100% confluency. Then one well of each cell line was subjected to one of the following pre-treatment before contacting with serum sample:
1) Control: no pre-treatment
2) 10 uM Brefeldin: cells were treated with 10 uM Brefeldin for 30 min at 37° C.
3) 36 uM Brefeldin: cells were treated with 36 uM Brefeldin for 30 min at 37° C.
4) Exo1: cells were treated with 100 uM Exo1 for 30 min at 37° C.
5) 10 uM Phenylarsine oxide: no pre-treatment, but 10 uM Phenylarsine oxide was added in subsequent 1 hr serum incubation
6) 50 uM Phenylarsine oxide: no pre-treatment, but 50 uM Phenylarsine oxide was added in subsequent 1 hr serum incubation
7) Jasplakinolide: cells were treated with 10 uM Jasplakinolide for 1 hr at 37° C.

After pre-treatment, cell medium was removed, cells were washed with PBS once and blocked with 1 mL 1% BSA in PBS for 30 min at 4° C. After blocking solution was removed, 0.5 mL PBS was added into each well for 1 hr incubation at 4° C. with continuous rocking. PBS control was for estimating the amount of FGF and VEGF secreted from cells and later bound to cells during 1 hr incubation. After 1 hr incubation, PBS was removed from cells and cells were rinsed with PBS three times before subjecting to ligand elution with 0.5 mL elution buffer (50 mM Glycine, 0.5M NaCl, pH 3.0) for 10 min at 4° C. The recovered elution buffer was then neutralized to pH 7.4.

The eluted samples were tested as per manufacturer's instructions using R&D Systems Duo-Set ELISA kits for FGF and VEGF. The samples were first diluted 1 to 3 in 1% BSA prepared in water before running in the ELISA. Table 7 shows various treatments either increase (positive number) or decrease (negative number) FGF and VEGF secretion from MDA-MB-231 and MRC5 cells in comparison to control without pre-treatment.

TABLE 7
The Effect of Exocytosis Inhibitors On Secretion of FGF And VEGF
From MDA-MB-231 And MRC5 Cells

| | FGF | | VEGF | |
|---|---|---|---|---|
| Treatment | MDA-MB-231 | MRC5 | MDA-MB-231 | MRC5 |
| 10 uM Brefeldin | 8% | −22% | 33% | −6% |
| 36 uM Brefedlin | 6% | −29% | 122% | 473% |
| Exo1 | ND | 71% | ND | −69% |
| 10 uM Phenylarsine | 0% | −58% | 19% | 66% |
| oxide | | | | |
| 50 uM Phenylarsine | 11% | −68% | −9% | 20% |
| oxide | | | | |
| Jasplakinolide | ND | 4% | ND | −33% |

### EXAMPLE 12

### Optimization of Ligand and Recovery Rate and Enrichment Efficiency

MDA-MB-231 cells were grown into confluency on a 6-well dish. After washed once with PBS, cells were fixed by 7.5% formalin followed by blocking with 1% BSA. After removing 1% BSA blocking solution, 0.5 mL neat serum spiked with EGF was added into each of three wells (well #1-3) and incubated at room temperature for 1 hr. Serum was then removed from each well. The well #1 was washed with 1 mL PBS for 1 minute once, well #2 was washed with the same condition twice and well #3 was washed three times. Elution buffer was then added into each well and incubated with ligand-bound cells for 10 minutes to elute ligands from cells. The ligand-containing elution buffer was then removed from cells and neutralized into pH 7.4. The neutralized elution buffer was used for quantification of EGF by ELISA and protein quantification by the methods described above.

TABLE 8
EGF Recovery and Enrichment Efficiency Under Different Washing
Conditions

| Washing | | EGF Enrichment |
|---|---|---|
| Condition | EGF Recovery Rate | Efficiency |
| 1 × 1 min | 60% | 117 |
| 2 × 1 min | 55% | 2830 |
| 3 × 1 min | 52% | 9100 |

Various modifications and processes to which the present invention may be applicable will be readily apparent to those of skill in the art to which the present invention is directed, upon review of the specification. Various references, publications, provisional and non-provisional United States or foreign patent applications, and/or United States or foreign patents, have been identified herein, each of which is incorporated herein in its entirety by this reference. Various aspects and features of the present invention have been explained or described in relation to understandings, beliefs, theories, underlying assumptions, and/or working or prophetic examples, although it will be understood that the invention is not bound to any particular understanding, belief, theory, underlying assumption, and/or working or prophetic example. Although the various aspects and features of the present invention have been described with respect to various embodiments and specific examples herein, it will be understood that the invention is entitled to protection within the full scope of the appended claims.

APPENDIX A

| Receptor Family | Gene | Receptor |
|---|---|---|
| Family 7TM\|A | ADMR | Receptor Adrenomedullin receptor |
| Family 7TM\|A | GPBAR1 | Receptor G-protein coupled bile acid receptor BG37 |
| Family 7TM\|A | GPR139 | Receptor G protein-coupled receptor 139 |
| Family 7TM\|A | GPR151 | Receptor G protein-coupled receptor 151 |
| Family 7TM\|A | GPR160 | Receptor G protein-coupled receptor 160 |
| Family 7TM\|A | GPR175 | Receptor GPR175 |
| Family 7TM\|A | GPR30 | Receptor G protein-coupled receptor 30 |
| Family 7TM\|A | GPR62 | Receptor G protein-coupled receptor 62 |
| Family 7TM\|A | GPR88 | Receptor G-protein coupled receptor 88 |
| Family 7TM\|A | NPSR1 | Receptor GPR154 isoform A |
| Family 7TM\|A | NPSR1 | Receptor GPR154 isoform B |
| Family 7TM\|A | P2RY8 | Receptor purinergic receptor P2Y, G-protein coupled, 8 |
| Family 7TM\|A\|Amine derivatives\|Adrenergic | ADRA1A | Receptor alpha-1A-adrenergic receptor, isoform 2 |
| Family 7TM\|A\|Amine derivatives\|Adrenergic | ADRA1B | Receptor alpha-1B-adrenergic receptor |
| Family 7TM\|A\|Amine derivatives\|Adrenergic | ADRA1D | Receptor alpha-1D-adrenergic receptor |
| Family 7TM\|A\|Amine derivatives\|Adrenergic | ADRA2A | Receptor alpha-2A-adrenergic receptor |
| Family 7TM\|A\|Amine derivatives\|Adrenergic | ADRA2B | Receptor alpha-2B-adrenergic receptor |
| Family 7TM\|A\|Amine derivatives\|Adrenergic | ADRA2C | Receptor adrenergic, alpha-2C receptor |
| Family 7TM\|A\|Amine derivatives\|Adrenergic | ADRB1 | Receptor beta-1-adrenergic receptor |
| Family 7TM\|A\|Amine derivatives\|Adrenergic | ADRB2 | Receptor adrenergic, beta-2-, receptor, surface |
| Family 7TM\|A\|Amine derivatives\|Adrenergic | ADRB3 | Receptor adrenergic, beta-3-, receptor |
| Family 7TM\|A\|Amine derivatives\|Adrenergic | GPR161 | Receptor G protein-coupled receptor 161 |
| Family 7TM\|A\|Amine derivatives\|Cholinergic/muscarinic | CHRM1 | Receptor cholinergic receptor, muscarinic 1 |
| Family 7TM\|A\|Amine derivatives\|Cholinergic/muscarinic | CHRM2 | Receptor cholinergic receptor, muscarinic 2 |
| Family 7TM\|A\|Amine derivatives\|Cholinergic/muscarinic | CHRM3 | Receptor cholinergic receptor, muscarinic 3 |
| Family 7TM\|A\|Amine derivatives\|Cholinergic/muscarinic | CHRM4 | Receptor cholinergic receptor, muscarinic 4 |
| Family 7TM\|A\|Amine derivatives\|Cholinergic/muscarinic | CHRM5 | Receptor cholinergic receptor, muscarinic 5 |
| Family 7TM\|A\|Amine derivatives\|Dopamine | DRD1 | Receptor dopamine receptor D1 |
| Family 7TM\|A\|Amine derivatives\|Dopamine | DRD2 | Receptor dopamine receptor D2 isoform long |
| Family 7TM\|A\|Amine derivatives\|Dopamine | DRD3 | Receptor dopamine receptor D3, isoform a |
| Family 7TM\|A\|Amine derivatives\|Dopamine | DRD4 | Receptor dopamine receptor D4 |
| Family 7TM\|A\|Amine derivatives\|Dopamine | DRD5 | Receptor DOPAMINE RECEPTOR D5 |
| Family 7TM\|A\|Amine derivatives\|Histamine | GPR52 | Receptor G protein-coupled receptor 52 |
| Family 7TM\|A\|Amine derivatives\|Histamine | HRH1 | Receptor histamine receptor H1 |
| Family 7TM\|A\|Amine derivatives\|Histamine | HRH2 | Receptor histamine receptor H2 |
| Family 7TM\|A\|Amine derivatives\|Histamine | HRH3 | Receptor histamine receptor H3 |
| Family 7TM\|A\|Amine derivatives\|Histamine | HRH4 | Receptor histamine H4 receptor |
| Family 7TM\|A\|Amine derivatives\|Melatonin | GPR50 | Receptor G protein-coupled receptor 50 |
| Family 7TM\|A\|Amine derivatives\|Melatonin | MTNR1A | Receptor melatonin receptor 1A |
| Family 7TM\|A\|Amine derivatives\|Melatonin | MTNR1B | Receptor melatonin receptor 1B |
| Family 7TM\|A\|Amine derivatives\|Other Amine Derivatives | GPR84 | Receptor GPR84 |
| Family 7TM\|A\|Amine derivatives\|Serotonin | GPR119 | Receptor similar to beta-2-andrenergic receptor |
| Family 7TM\|A\|Amine derivatives\|Serotonin | GPR26 | Receptor G protein-coupled receptor 26 |
| Family 7TM\|A\|Amine derivatives\|Serotonin | GPR78 | Receptor G protein-coupled receptor 78 |
| Family 7TM\|A\|Amine derivatives\|Serotonin | HTR1A | Receptor 5-hydroxytryptamine (serotonin) receptor 1A |
| Family 7TM\|A\|Amine derivatives\|Serotonin | HTR1B | Receptor 5-hydroxytryptamine (serotonin) receptor 1B |
| Family 7TM\|A\|Amine derivatives\|Serotonin | HTR1D | Receptor 5-hydroxytryptamine (serotonin) receptor 1D |
| Family 7TM\|A\|Amine derivatives\|Serotonin | HTR1E | Receptor 5-hydroxytryptamine (serotonin) receptor 1E |
| Family 7TM\|A\|Amine derivatives\|Serotonin | HTR1F | Receptor 5-hydroxytryptamine (serotonin) receptor 1F |
| Family 7TM\|A\|Amine derivatives\|Serotonin | HTR2A | Receptor 5-hydroxytryptamine (serotonin) receptor 2A |
| Family 7TM\|A\|Amine derivatives\|Serotonin | HTR2B | Receptor 5-hydroxytryptamine (serotonin) receptor 2B |
| Family 7TM\|A\|Amine derivatives\|Serotonin | HTR2C | Receptor 5-hydroxytryptamine (serotonin) receptor 2C |
| Family 7TM\|A\|Amine derivatives\|Serotonin | HTR3A | Receptor 5-hydroxytryptamine (serotonin) receptor 3A |
| Family 7TM\|A\|Amine derivatives\|Serotonin | HTR3B | Receptor 5-hydroxytryptamine (serotonin) receptor 3B |
| Family 7TM\|A\|Amine derivatives\|Serotonin | HTR3C | Receptor 5-hydroxytryptamine (serotonin) receptor 3, family member C |
| Family 7TM\|A\|Amine derivatives\|Serotonin | HTR3E | Receptor 5-hydroxytryptamine serotonin receptor 3E isoform a |
| Family 7TM\|A\|Amine derivatives\|Serotonin | HTR4 | Receptor 5-hydroxytryptamine (serotonin) receptor 4 |
| Family 7TM\|A\|Amine derivatives\|Serotonin | HTR5A | Receptor 5-hydroxytryptamine (serotonin) receptor 5A |
| Family 7TM\|A\|Amine derivatives\|Serotonin | HTR5B | Receptor 5-hydroxytryptamine (serotonin) receptor 5B |
| Family 7TM\|A\|Amine derivatives\|Serotonin | HTR6 | Receptor 5-hydroxytryptamine (serotonin) receptor 6 |
| Family 7TM\|A\|Amine derivatives\|Serotonin | HTR7 | Receptor 5-hydroxytryptamine (serotonin) receptor 7, isoform d |
| Family 7TM\|A\|Amine derivatives\|Trace | LOC442259 | Receptor LOC442259 |
| Family 7TM\|A\|Amine derivatives\|Trace | TAAR1 | Receptor trace amine receptor 1 |
| Family 7TM\|A\|Amine derivatives\|Trace | TAAR2 | Receptor G protein-coupled receptor 58 |
| Family 7TM\|A\|Amine derivatives\|Trace | TAAR3 | Receptor trace amine associated receptor 3 |
| Family 7TM\|A\|Amine derivatives\|Trace | TAAR5 | Receptor putative neurotransmitter receptor |
| Family 7TM\|A\|Amine derivatives\|Trace | TAAR6 | Receptor trace amine receptor 4 (TA4) gene, complete cds |
| Family 7TM\|A\|Amine derivatives\|Trace | TAAR8 | Receptor G protein-coupled receptor 102 |
| Family 7TM\|A\|Amine derivatives\|Trace | TAAR9 | Receptor trace amine receptor 3 |
| Family 7TM\|A\|Light\|Opsin | RGR | Receptor retinal G protein coupled receptor |
| Family 7TM\|A\|Lipid | GPR63 | Receptor G protein-coupled receptor 63 |
| Family 7TM\|A\|Lipid\|Cysteinyl_leukotriene/EBV- | CYSLTR1 | Receptor cysteinyl leukotriene receptor 1 |
| induced/platelet_activating_factor | | |
| Family 7TM\|A\|Lipid\|Cysteinyl_leukotriene/EBV- | CYSLTR2 | Receptor cysteinyl leukotriene CysLT2 receptor |
| induced/platelet_activating_factor | | |
| Family 7TM\|A\|Lipid\|Cysteinyl_leukotriene/EBV- | EBI2 | Receptor EBV-induced G protein-coupled receptor 2 |
| induced/platelet_activating_factor | | |
| Family 7TM\|A\|Lipid\|Cysteinyl_leukotriene/EBV- | GPR135 | Receptor similar to putative leukocyte platelet-activating factor receptor |
| induced/platelet_activating_factor | | |
| Family 7TM\|A\|Lipid\|Cysteinyl_leukotriene/EBV- | GPR171 | Receptor GPR171 |
| induced/platelet_activating_factor | | |
| Family 7TM\|A\|Lipid\|Cysteinyl_leukotriene/EBV- | GPR34 | Receptor G protein-coupled receptor 34 |
| induced/platelet_activating_factor | | |
| Family 7TM\|A\|Lipid\|Cysteinyl_leukotriene/EBV- | PTAFR | Receptor platelet-activating factor receptor |
| induced/platelet_activating_factor | | |
| Family 7TM\|A\|Lipid\|Fatty_acid | FFAR1 | Receptor G protein-coupled receptor 40 |
| Family 7TM\|A\|Lipid\|Fatty_acid | FFAR2 | Receptor G protein-coupled receptor 43 |
| Family 7TM\|A\|Lipid\|Fatty_acid | FFAR3 | Receptor G protein-coupled receptor 41 |
| Family 7TM\|A\|Lipid\|Fatty_acid | GPR12 | Receptor G protein-coupled receptor 12 |
| Family 7TM\|A\|Lipid\|Fatty_acid | GPR120 | Receptor G PROTEIN-COUPLED RECEPTOR 120 |
| Family 7TM\|A\|Lipid\|Fatty_acid | GPR3 | Receptor G protein-coupled receptor 3 |
| Family 7TM\|A\|Lipid\|Fatty_acid | GPR42 | Receptor G protein-coupled receptor 42 |
| Family 7TM\|A\|Lipid\|Fatty_acid | GPR6 | Receptor G protein-coupled receptor 6 |
| Family 7TM\|A\|Lipid\|Leukotriene_B4 | LTB4R | Receptor leukotriene B4 receptor |
| Family 7TM\|A\|Lipid\|Leukotriene_B4 | LTB4R2 | Receptor leukotriene B4 receptor 2 |
| Family 7TM\|A\|Lipid\|Lysosphingolipid/lysophosphatidic_acid/EDG | CNR1 | Receptor central cannabinoid receptor, isoform a |
| Family 7TM\|A\|Lipid\|Lysosphingolipid/lysophosphatidic_acid/EDG | CNR2 | Receptor cannabinoid receptor 2 (macrophage) |
| Family 7TM\|A\|Lipid\|Lysosphingolipid/lysophosphatidic_acid/EDG | EDG1 | Receptor Probable G protein-coupled receptor EDG-1 |
| Family 7TM\|A\|Lipid\|Lysosphingolipid/lysophosphatidic_acid/EDG | EDG2 | Receptor Lysophosphatidic acid receptor Edg-2 |
| Family 7TM\|A\|Lipid\|Lysosphingolipid/lysophosphatidic_acid/EDG | EDG3 | Receptor Lysosphingolipid receptor |
| Family 7TM\|A\|Lipid\|Lysosphingolipid/lysophosphatidic_acid/EDG | EDG4 | Receptor Lysophosphatidic acid receptor Edg-4 |
| Family 7TM\|A\|Lipid\|Lysosphingolipid/lysophosphatidic_acid/EDG | EDG5 | Receptor ENDOTHELIAL DIFFERENTIATION GENE 5 |
| Family 7TM\|A\|Lipid\|Lysosphingolipid/lysophosphatidic_acid/EDG | EDG6 | Receptor endothelial differentiation, G protein coupled receptor 6 |
| | | precursor |
| Family 7TM\|A\|Lipid\|Lysosphingolipid/lysophosphatidic_acid/EDG | EDG7 | Receptor Lysophosphatidic acid receptor Edg-7 |
| Family 7TM\|A\|Lipid\|Lysosphingolipid/lysophosphatidic_acid/EDG | EDG8 | Receptor Sphingosine 1-phosphate receptor Edg-8 |
| Family 7TM\|A\|Lipid\|Lysosphingolipid/lysophosphatidic_acid/EDG | GPR45 | Receptor G protein-coupled receptor 45 |
| Family 7TM\|A\|Lipid\|Prostanoid/proataglandin/thromboxane/prostacycline | PTGDR | Receptor prostaglandin D2 receptor (DP) |
| Family 7TM\|A\|Lipid\|Prostanoid/proataglandin/thromboxane/prostacycline | PTGER1 | Receptor prostaglandin E receptor 1 (subtype EP1), 42 kDa |
| Family 7TM\|A\|Lipid\|Prostanoid/proataglandin/thromboxane/prostacycline | PTGER2 | Receptor prostaglandin E receptor 2 (subtype EP2), 53 kDa |
| Family 7TM\|A\|Lipid\|Prostanoid/proataglandin/thromboxane/prostacycline | PTGER3 | Receptor prostaglandin E receptor 3 (subtype EP3) |
| Family 7TM\|A\|Lipid\|Prostanoid/proataglandin/thromboxane/prostacycline | PTGER4 | Receptor prostaglandin E receptor 4 (subtype EP4) |
| Family 7TM\|A\|Lipid\|Prostanoid/proataglandin/thromboxane/prostacycline | PTGFR | Receptor prostaglandin F receptor (FP) |
| Family 7TM\|A\|Lipid\|Prostanoid/proataglandin/thromboxane/prostacycline | PTGIR | Receptor prostaglandin I2 (prostacyclin) receptor (IP) |
| Family 7TM\|A\|Lipid\|Prostanoid/proataglandin/thromboxane/prostacycline | TBXA1R | Receptor thromboxane A1 receptor |
| Family 7TM\|A\|Lipid\|Prostanoid/proataglandin/thromboxane/prostacycline | TBXA2R | Receptor thromboxane A2 receptor |
| Family 7TM\|A\|MAS-related | MAS-related G | Receptor MAS-related G protein-coupled receptor MRGF |
| | protein-coupled | |
| | receptor MRGF | |
| Family 7TM\|A\|MAS-related | MRGPRE | Receptor similar to MrgE G protein-coupled receptor |
| Family 7TM\|A\|MAS-related | MRGPRX1 | Receptor G protein-coupled receptor MRGX1 |
| Family 7TM\|A\|MAS-related | MRGPRX2 | Receptor G protein-coupled receptor MRGX2 |
| Family 7TM\|A\|MAS-related | MRGPRX3 | Receptor G protein-coupled receptor MRGX3 |
| Family 7TM\|A\|MAS-related | MRGPRX4 | Receptor G protein-coupled receptor MRGX4 |
| Family 7TM\|A\|Nucleotide\|Adenosine | ADORA1 | Receptor adenosine A1 receptor |
| Family 7TM\|A\|Nucleotide\|Adenosine | ADORA2A | Receptor adenosine A2a receptor |
| Family 7TM\|A\|Nucleotide\|Adenosine | ADORA2B | Receptor adenosine A2b receptor |
| Family 7TM\|A\|Nucleotide\|Adenosine | ADORA2L1 | Receptor adenosine A2 receptor-like 1 |
| Family 7TM\|A\|Nucleotide\|Adenosine | ADORA3 | Receptor adenosine A3 receptor |
| Family 7TM\|A\|Nucleotide\|Purinergic/uridine | GPR17 | Receptor G protein-coupled receptor 17 |
| Family 7TM\|A\|Nucleotide\|Purinergic/uridine | GPR174 | Receptor putative purinergic receptor FKSG79 |
| Family 7TM\|A\|Nucleotide\|Purinergic/uridine | GPR18 | Receptor G protein-coupled receptor 18 |
| Family 7TM\|A\|Nucleotide\|Purinergic/uridine | GPR20 | Receptor G protein-coupled receptor 20 |
| Family 7TM\|A\|Nucleotide\|Purinergic/uridine | GPR23 | Receptor GPR23 |
| Family 7TM\|A\|Nucleotide\|Purinergic/uridine | GPR55 | Receptor G protein-coupled receptor 55 |
| Family 7TM\|A\|Nucleotide\|Purinergic/uridine | GPR87 | Receptor G protein-coupled receptor 87 |
| Family 7TM\|A\|Nucleotide\|Purinergic/uridine | GPR92 | Receptor putative G protein-coupled receptor 92 |
| Family 7TM\|A\|Nucleotide\|Purinergic/uridine | OXGR1 | Receptor G protein-coupled receptor 80 |
| Family 7TM\|A\|Nucleotide\|Purinergic/uridine | P2RY1 | Receptor purinergic receptor P2Y1 |
| Family 7TM\|A\|Nucleotide\|Purinergic/uridine | P2RY10 | Receptor putative purinergic receptor P2Y10 |
| Family 7TM\|A\|Nucleotide\|Purinergic/uridine | P2RY11 | Receptor purinergic receptor P2Y, G-protein coupled, 11 |
| Family 7TM\|A\|Nucleotide\|Purinergic/uridine | P2RY12 | Receptor Purinergic receptor P2Y, G protein-coupled, 12 |
| Family 7TM\|A\|Nucleotide\|Purinergic/uridine | P2RY13 | Receptor G protein-coupled receptor 86 |
| Family 7TM\|A\|Nucleotide\|Purinergic/uridine | P2RY2 | Receptor purinergic receptor P2Y, G-protein coupled, 2 |
| Family 7TM\|A\|Nucleotide\|Purinergic/uridine | P2RY4 | Receptor pyrimidinergic receptor P2Y4 |
| Family 7TM\|A\|Nucleotide\|Purinergic/uridine | P2RY6 | Receptor pyrimidinergic receptor P2Y, G-protein coupled, 6 |
| Family 7TM\|A\|Nucleotide\|Purinergic/uridine | SUCNR1 | Receptor G protein-coupled receptor 91 |
| Family 7TM\|A\|Nucleotide\|UDP-glucose | GPR22 | Receptor G protein-coupled receptor 22 |
| Family 7TM\|A\|Nucleotide\|UDP-glucose | P2RY14 | Receptor G protein-coupled receptor 105 |
| Family 7TM\|A\|OGR1/GPR4 | GPR132 | Receptor G protein-coupled receptor G2A |
| Family 7TM\|A\|OGR1/GPR4 | GPR4 | Receptor G protein-coupled receptor 4 |
| Family 7TM\|A\|OGR1/GPR4 | GPR65 | Receptor G protein-coupled receptor 65 |
| Family 7TM\|A\|OGR1/GPR4 | GPR68 | Receptor G protein-coupled receptor 68 |
| Family 7TM\|A\|Orphan | GPR101 | Receptor G protein-coupled receptor 101 |
| Family 7TM\|A\|Orphan | GPR146 | Receptor G protein-coupled receptor 146 |
| Family 7TM\|A\|Orphan | GPR148 | Receptor GPR148 |
| Family 7TM\|A\|Orphan | GPR152 | Receptor G protein-coupled receptor 152 |
| Family 7TM\|A\|Orphan | GPR21 | Receptor G protein-coupled receptor 21 |
| Family 7TM\|A\|Orphan | GPR61 | Receptor G protein-coupled receptor 61 |
| Family 7TM\|A\|Orphan | P2RY5 | Receptor purinergic receptor P2Y, G-protein coupled, 5 |
| Family 7TM\|A\|Polypeptide | DARC | Receptor Duffy blood group |
| Family 7TM\|A\|Polypeptide | GPR150 | Receptor G protein-coupled receptor 150 |
| Family 7TM\|A\|Polypeptide\|Apelin/angiotensin/bradykinin | AGTR1 | Receptor angiotensin II receptor, type 1 |
| Family 7TM\|A\|Polypeptide\|Apelin/angiotensin/bradykinin | AGTR2 | Receptor angiotensin II receptor, type 2 |
| Family 7TM\|A\|Polypeptide\|Apelin/angiotensin/bradykinin | AGTRL1 | Receptor angiotensin II receptor-like 1 |
| Family 7TM\|A\|Polypeptide\|Apelin/angiotensin/bradykinin | BDKRB1 | Receptor bradykinin receptor B1 |
| Family 7TM\|A\|Polypeptide\|Apelin/angiotensin/bradykinin | BDKRB2 | Receptor bradykinin receptor B2 |
| Family 7TM\|A\|Polypeptide\|Apelin/angiotensin/bradykinin | GPR142 | Receptor G protein-coupled receptor 142 |
| Family 7TM\|A\|Polypeptide\|Apelin/angiotensin/bradykinin | GPR15 | Receptor G protein-coupled receptor 15 |
| Family 7TM\|A\|Polypeptide\|Apelin/angiotensin/bradykinin | GPR25 | Receptor G protein-coupled receptor 25 |
| Family 7TM\|A\|Polypeptide\|Apelin/angiotensin/bradykinin | RXFP3 | Receptor G-protein coupled receptor SALPR |
| Family 7TM\|A\|Polypeptide\|Apelin/angiotensin/bradykinin | RXFP4 | Receptor relaxin 3 receptor 2 |
| Family 7TM\|A\|Polypeptide\|Chemokine/interleukin | AMFR | Receptor autocrine motility factor receptor isoform a |
| Family 7TM\|A\|Polypeptide\|Chemokine/interleukin | BLR1 | Receptor Burkitt lymphoma receptor 1 isoform 1 |
| Family 7TM\|A\|Polypeptide\|Chemokine/interleukin | C5AR1 | Receptor complement component 5 receptor 1 (C5a ligand) |
| Family 7TM\|A\|Polypeptide\|Chemokine/interleukin | CCR1 | Receptor chemokine (C-C motif) receptor 1 |
| Family 7TM\|A\|Polypeptide\|Chemokine/interleukin | CCR10 | Receptor CC chemokine receptor 10 |
| Family 7TM\|A\|Polypeptide\|Chemokine/interleukin | CCR2 | Receptor chemokine (C-C motif) receptor 2, isoform A |
| Family 7TM\|A\|Polypeptide\|Chemokine/interleukin | CCR3 | Receptor chemokine (C-C motif) receptor 3 |
| Family 7TM\|A\|Polypeptide\|Chemokine/interleukin | CCR4 | Receptor chemokine (C-C motif) receptor 4 |
| Family 7TM\|A\|Polypeptide\|Chemokine/interleukin | CCR5 | Receptor chemokine (C-C motif) receptor 5 |
| Family 7TM\|A\|Polypeptide\|Chemokine/interleukin | CCR6 | Receptor chemokine (C-C motif) receptor 6 |
| Family 7TM\|A\|Polypeptide\|Chemokine/interleukin | CCR7 | Receptor chemokine (C-C motif) receptor 7 |
| Family 7TM\|A\|Polypeptide\|Chemokine/interleukin | CCR8 | Receptor chemokine (C-C motif) receptor 8 |
| Family 7TM\|A\|Polypeptide\|Chemokine/interleukin | CCR9 | Receptor chemokine (C-C motif) receptor 9 isoform A |
| Family 7TM\|A\|Polypeptide\|Chemokine/interleukin | CCRL1 | Receptor orphan seven-transmembrane receptor, chemokine related |
| Family 7TM\|A\|Polypeptide\|Chemokine/interleukin | CCRL2 | Receptor chemokine (C-C motif) receptor-like 2 |
| Family 7TM\|A\|Polypeptide\|Chemokine/interleukin | CMKLR1 | Receptor chemokine-like receptor 1 |
| Family 7TM\|A\|Polypeptide\|Chemokine/interleukin | CMKOR1 | Receptor similar to G protein-coupled receptor RDC1 homolog |
| Family 7TM\|A\|Polypeptide\|Chemokine/interleukin | CX3CR1 | Receptor chemokine (C-X3-C motif) receptor 1 |
| Family 7TM\|A\|Polypeptide\|Chemokine/interleukin | CXCR3 | Receptor chemokine (C-X-C motif) receptor 3 |
| Family 7TM\|A\|Polypeptide\|Chemokine/interleukin | CXCR4 | Receptor chemokine (C-X-C motif) receptor 4 |
| Family 7TM\|A\|Polypeptide\|Chemokine/interleukin | CXCR6 | Receptor G protein-coupled receptor TYMSTR |
| Family 7TM\|A\|Polypeptide\|Chemokine/interleukin | GPR1 | Receptor G protein-coupled receptor 1 |
| Family 7TM\|A\|Polypeptide\|Chemokine/interleukin | GPR109A | Receptor G protein-coupled receptor 109A |
| Family 7TM\|A\|Polypeptide\|Chemokine/interleukin | GPR109B | Receptor G protein-coupled receptor 109B |
| Family 7TM\|A\|Polypeptide\|Chemokine/interleukin | GPR31 | Receptor G protein-coupled receptor 31 |
| Family 7TM\|A\|Polypeptide\|Chemokine/interleukin | GPR35 | Receptor G protein-coupled receptor 35 |
| Family 7TM\|A\|Polypeptide\|Chemokine/interleukin | GPR44 | Receptor GPR44 |
| Family 7TM\|A\|Polypeptide\|Chemokine/interleukin | GPR77 | Receptor G protein-coupled receptor C5L2 |
| Family 7TM\|A\|Polypeptide\|Chemokine/interleukin | GPR81 | Receptor G protein-coupled receptor 81 |
| Family 7TM\|A\|Polypeptide\|Chemokine/interleukin | IL8RA | Receptor interleukin 8 receptor, alpha |
| Family 7TM\|A\|Polypeptide\|Chemokine/interleukin | IL8RB | Receptor interleukin 8 receptor, beta |
| Family 7TM\|A\|Polypeptide\|Chemokine/interleukin | OXER1 | Receptor G-protein coupled receptor TG1019 |
| Family 7TM\|A\|Polypeptide\|Chemokine/interleukin | XCR1 | Receptor G protein-coupled receptor 5 |
| Family 7TM\|A\|Polypeptide\|Endothelin/CCK/Gastrin releasing peptide | BRS3 | Receptor bombesin-like receptor 3 |
| Family 7TM\|A\|Polypeptide\|Endothelin/CCK/Gastrin releasing peptide | CCKAR | Receptor cholecystokinin A receptor |
| Family 7TM\|A\|Polypeptide\|Endothelin/CCK/Gastrin releasing peptide | CCKBR | Receptor cholecystokinin B receptor |
| Family 7TM\|A\|Polypeptide\|Endothelin/CCK/Gastrin releasing peptide | EDNRA | Receptor endothelin receptor type A |
| Family 7TM\|A\|Polypeptide\|Endothelin/CCK/Gastrin releasing peptide | EDNRB | Receptor endothelin receptor type B, isoform 1 |
| Family 7TM\|A\|Polypeptide\|Endothelin/CCK/Gastrin releasing peptide | GPR37 | Receptor GPR37 |
| Family 7TM\|A\|Polypeptide\|Endothelin/CCK/Gastrin releasing peptide | GPR37L1 | Receptor G-protein coupled receptor 37 like 1 |
| Family 7TM\|A\|Polypeptide\|Endothelin/CCK/Gastrin releasing peptide | GRPR | Receptor gastrin-releasing peptide receptor |
| Family 7TM\|A\|Polypeptide\|Endothelin/CCK/Gastrin releasing peptide | NMBR | Receptor neuromedin B receptor |
| Family 7TM\|A\|Polypeptide\|Formyl | C3AR1 | Receptor complement component 3a receptor 1 |
| Family 7TM\|A\|Polypeptide\|Formyl | FPR1 | Receptor formyl peptide receptor 1 |
| Family 7TM\|A\|Polypeptide\|Formyl | FPRL1 | Receptor formyl peptide receptor-like 1 |
| Family 7TM\|A\|Polypeptide\|Formyl | FPRL2 | Receptor formyl peptide receptor-like 2 |
| Family 7TM\|A\|Polypeptide\|Formyl | GPR32 | Receptor G protein-coupled receptor 32 |
| Family 7TM\|A\|Polypeptide\|Galanin/kisspeptin/urotensin | GALR1 | Receptor galanin receptor 1 |
| Family 7TM\|A\|Polypeptide\|Galanin/kisspeptin/urotensin | GALR2 | Receptor galanin receptor 2 |
| Family 7TM\|A\|Polypeptide\|Galanin/kisspeptin/urotensin | GALR3 | Receptor galanin receptor 3 |
| Family 7TM\|A\|Polypeptide\|Galanin/kisspeptin/urotensin | KISS1R | Receptor GPR54 |
| Family 7TM\|A\|Polypeptide\|Galanin/kisspeptin/urotensin | UTS2R | Receptor GPR14 |
| Family 7TM\|A\|Polypeptide\|Gonadotropin-releasing | GNRHR | Receptor gonadotropin-releasing hormone receptor |
| Family 7TM\|A\|Polypeptide\|Gonadotropin-releasing | GNRHR2 | Receptor gonadotropin-releasing hormone (type 2) receptor 2 |
| Family 7TM\|A\|Polypeptide\|LGR (glycoprotein hormones, relaxin- | FSHR | Receptor follicle stimulating hormone receptor |
| like) | | |
| Family 7TM\|A\|Polypeptide\|LGR (glycoprotein hormones, relaxin- | LGR4 | Receptor G protein-coupled receptor 48 |
| like) | | |
| Family 7TM\|A\|Polypeptide\|LGR (glycoprotein hormones, relaxin- | LGR5 | Receptor G protein-coupled receptor 49 |
| like) | | |
| Family 7TM\|A\|Polypeptide\|LGR (glycoprotein hormones, relaxin- | LGR6 | Receptor leucine-rich repeat-containing G protein-coupled receptor 6 |
| like) | | |
| Family 7TM\|A\|Polypeptide\|LGR (glycoprotein hormones, relaxin- | LHCGR | Receptor luteinizing hormone/choriogonadotropin receptor precursor |
| like) | | |
| Family 7TM\|A\|Polypeptide\|LGR (glycoprotein hormones, relaxin- | RXFP1 | Receptor leucine-rich repeat-containing G protein-coupled receptor 7 |
| like) | | |
| Family 7TM\|A\|Polypeptide\|LGR (glycoprotein hormones, relaxin- | RXFP2 | Receptor leucine-rich repeat-containing G protein-coupled receptor 8 |
| like) | | |
| Family 7TM\|A\|Polypeptide\|LGR (glycoprotein hormones, relaxin- | TSHR | Receptor thyroid stimulating hormone receptor |
| like) | | |
| Family 7TM\|A\|Polypeptide\|MCHR | GPR141 | Receptor Similiar to chemokine receptor |
| Family 7TM\|A\|Polypeptide\|MCHR | GPR82 | Receptor G protein-coupled receptor 82 |
| Family 7TM\|A\|Polypeptide\|MCHR | MCHR1 | Receptor MCHR1 |
| Family 7TM\|A\|Polypeptide\|MCHR | MCHR2 | Receptor MCHR2 |
| Family 7TM\|A\|Polypeptide\|Melanocortin/ACTH/MSH | MC1R | Receptor melanocortin 1 receptor |
| Family 7TM\|A\|Polypeptide\|Melanocortin/ACTH/MSH | MC2R | Receptor melanocortin 2 receptor |
| Family 7TM\|A\|Polypeptide\|Melanocortin/ACTH/MSH | MC3R | Receptor melanocortin 3 receptor |
| Family 7TM\|A\|Polypeptide\|Melanocortin/ACTH/MSH | MC4R | Receptor melanocortin 4 receptor |
| Family 7TM\|A\|Polypeptide\|Melanocortin/ACTH/MSH | MC5R | Receptor melanocortin 5 receptor |
| Family 7TM\|A\|Polypeptide\|Neuromedin | GHSR | Receptor growth hormone secretagogue receptor |
| U/neurotensin/ghrelin/thyrotropin-releasing | | |
| Family 7TM\|A\|Polypeptide\|Neuromedin | GPR39 | Receptor G protein-coupled receptor 39 |
| U/neurotensin/ghrelin/thyrotropin-releasing | | |
| Family 7TM\|A\|Polypeptide\|Neuromedin | MLNR | Receptor G protein-coupled receptor 38 |
| U/neurotensin/ghrelin/thyrotropin-releasing | | |
| Family 7TM\|A\|Polypeptide\|Neuromedin | NMUR1 | Receptor neuromedin U receptor 1 |
| U/neurotensin/ghrelin/thyrotropin-releasing | | |
| Family 7TM\|A\|Polypeptide\|Neuromedin | NMUR2 | Receptor neuromedin U receptor 2 |
| U/neurotensin/ghrelin/thyrotropin-releasing | | |
| Family 7TM\|A\|Polypeptide\|Neuromedin | NTSR1 | Receptor neurotensin receptor 1 |
| U/neurotensin/ghrelin/thyrotropin-releasing | | |
| Family 7TM\|A\|Polypeptide\|Neuromedin | NTSR2 | Receptor neurotensin receptor 2 |
| U/neurotensin/ghrelin/thyrotropin-releasing | | |
| Family 7TM\|A\|Polypeptide\|Neuromedin | TRHR | Receptor thyrotropin-releasing hormone receptor |
| U/neurotensin/ghrelin/thyrotropin-releasing | | |
| Family 7TM\|A\|Polypeptide\|NeuropeptideY_tachykinin_orexin_pancreatic_peptide | GPR176 | Receptor G protein-coupled receptor 176 |
| Family 7TM\|A\|Polypeptide\|NeuropeptideY_tachykinin_orexin_pancreatic_peptide | GPR19 | Receptor G protein-coupled receptor 19 |
| Family 7TM\|A\|Polypeptide\|NeuropeptideY_tachykinin_orexin_pancreatic_peptide | GPR83 | Receptor G protein-coupled receptor 83 |
| Family 7TM\|A\|Polypeptide\|NeuropeptideY_tachykinin_orexin_pancreatic_peptide | HCRTR1 | Receptor orexin receptor 1 |
| Family 7TM\|A\|Polypeptide\|NeuropeptideY_tachykinin_orexin_pancreatic_peptide | HCRTR2 | Receptor orexin receptor 2 |
| Family 7TM\|A\|Polypeptide\|NeuropeptideY_tachykinin_orexin_pancreatic_peptide | NPFFR1 | Receptor GPR147 |
| Family 7TM\|A\|Polypeptide\|NeuropeptideY_tachykinin_orexin_pancreatic_peptide | NPFFR2 | Receptor GPR74 |
| Family 7TM\|A\|Polypeptide\|NeuropeptideY_tachykinin_orexin_pancreatic_peptide | NPY1R | Receptor neuropeptide Y receptor Y1 |
| Family 7TM\|A\|Polypeptide\|NeuropeptideY_tachykinin_orexin_pancreatic_peptide | NPY2R | Receptor neuropeptide Y receptor Y2 |
| Family 7TM\|A\|Polypeptide\|NeuropeptideY_tachykinin_orexin_pancreatic_peptide | NPY5R | Receptor neuropeptide Y receptor Y5 |
| Family 7TM\|A\|Polypeptide\|NeuropeptideY_tachykinin_orexin_pancreatic_peptide | NPY6R | Receptor Truncated pancreatic polypeptide receptor PP2 |
| Family 7TM\|A\|Polypeptide\|NeuropeptideY_tachykinin_orexin_pancreatic_peptide | PPYR1 | Receptor pancreatic polypeptide receptor 1 |
| Family 7TM\|A\|Polypeptide\|NeuropeptideY_tachykinin_orexin_pancreatic_peptide | PRLHR | Receptor G protein-coupled receptor 10 |
| | PROKR1 | Receptor G protein-coupled receptor 73 |
| Family 7TM\|A\|Polypeptide\|NeuropeptideY_tachykinin_orexin_pancreatic_peptide | PROKR2 | Receptor G protein-coupled receptor 73-like 1 |
| Family 7TM\|A\|Polypeptide\|NeuropeptideY_tachykinin_orexin_pancreatic_peptide | TACR1 | Receptor tachykinin receptor 1 isoform long |
| Family 7TM\|A\|Polypeptide\|NeuropeptideY_tachykinin_orexin_pancreatic_peptide | TACR2 | Receptor tachykinin receptor 2 |
| Family 7TM\|A\|Polypeptide\|NeuropeptideY_tachykinin_orexin_pancreatic_peptide | TACR3 | Receptor tachykinin receptor 3 |
| Family 7TM\|A\|Polypeptide\|Proteinase-activated/thrombin | F2R | Receptor coagulation factor II (thrombin) receptor |
| Family 7TM\|A\|Polypeptide\|Proteinase-activated/thrombin | F2RL1 | Receptor coagulation factor II (thrombin) receptor-like 1 precursor |
| Family 7TM\|A\|Polypeptide\|Proteinase-activated/thrombin | F2RL2 | Receptor coagulation factor II (thrombin) receptor-like 2 precursor |
| Family 7TM\|A\|Polypeptide\|Proteinase-activated/thrombin | F2RL3 | Receptor coagulation factor II (thrombin) receptor-like 3 |
| Family 7TM\|A\|Polypeptide\|Somatostatin&OPRL | Delta-type | Receptor Delta-type opioid receptor (DOR-1) |
| | opioid receptor | |
| | (DOR-1) | |
| Family 7TM\|A\|Polypeptide\|Somatostatin&OPRL | NPBWR1 | Receptor GPR7 |
| Family 7TM\|A\|Polypeptide\|Somatostatin&OPRL | NPBWR2 | Receptor G protein-coupled receptor 8 |
| Family 7TM\|A\|Polypeptide\|Somatostatin&OPRL | OPRK1 | Receptor opioid receptor, kappa 1 |
| Family 7TM\|A\|Polypeptide\|Somatostatin&OPRL | OPRL1 | Receptor opiate receptor-like 1 |
| Family 7TM\|A\|Polypeptide\|Somatostatin&OPRL | OPRM1 | Receptor opioid receptor, mu 1 |
| Family 7TM\|A\|Polypeptide\|Somatostatin&OPRL | SSTR1 | Receptor somatostatin receptor 1 |
| Family 7TM\|A\|Polypeptide\|Somatostatin&OPRL | SSTR2 | Receptor somatostatin receptor 2 |
| Family 7TM\|A\|Polypeptide\|Somatostatin&OPRL | SSTR3 | Receptor somatostatin receptor 3 |
| Family 7TM\|A\|Polypeptide\|Somatostatin&OPRL | SSTR4 | Receptor somatostatin receptor 4 |
| Family 7TM\|A\|Polypeptide\|Somatostatin&OPRL | SSTR5 | Receptor somatostatin receptor 5 |
| Family 7TM\|A\|Polypeptide\|SREB | GPR173 | Receptor G-protein coupled receptor 173 |
| Family 7TM\|A\|Polypeptide\|SREB | GPR27 | Receptor G protein-coupled receptor 27 |
| Family 7TM\|A\|Polypeptide\|SREB | GPR85 | Receptor G protein-coupled receptor 85 |
| Family 7TM\|A\|Polypeptide\|Vasopressin/oxytocin | AVPR1A | Receptor arginine vasopressin receptor 1A |
| Family 7TM\|A\|Polypeptide\|Vasopressin/oxytocin | AVPR1B | Receptor Arginine vasopressin receptor 1B |
| Family 7TM\|A\|Polypeptide\|Vasopressin/oxytocin | AVPR2 | Receptor arginine vasopressin receptor 2 |
| Family 7TM\|A\|Polypeptide\|Vasopressin/oxytocin | OXTR | Receptor oxytocin receptor |
| Family 7TM\|B | GPR123 | Receptor G protein-coupled receptor 123 |
| Family 7TM\|B | GPR128 | Receptor G protein-coupled receptor 128 |
| Family 7TM\|B | GPR133 | Receptor GPR133 |
| Family 7TM\|B | GPR143 | Receptor G protein-coupled receptor 143 |
| Family 7TM\|B | GPR144 | Receptor G protein-coupled receptor 144 |
| Family 7TM\|B | GPR97 | Receptor GPR97 |
| Family 7TM\|B\|Orphan | GPR110 | Receptor G-protein coupled receptor 110 isoform 1 |
| Family 7TM\|B\|Orphan\|CD97 | CELSR1 | Receptor cadherin EGF LAG seven-pass G-type receptor 1 |
| Family 7TM\|B\|Orphan\|CD97 | CELSR2 | Receptor cadherin EGF LAG seven-pass G-type receptor 2 |
| Family 7TM\|B\|Orphan\|CD97 | CELSR3 | Receptor cadherin EGF LAG seven-pass G-type receptor 3 |
| Family 7TM\|B\|Orphan\|CD97 | EMR1 | Receptor egf-like module containing, mucin-like, hormone receptor-like |
| | | sequence 1 |
| Family 7TM\|B\|Orphan\|CD97 | EMR2 | Receptor egf-like module containing, mucin-like, hormone receptor-like |
| | | sequence 2 isoform a |
| Family 7TM\|B\|Orphan\|CD97 | EMR3 | Receptor egf-like module-containing mucin-like receptor 3 isoform a |
| Family 7TM\|B\|Orphan\|CD97 | EMR4 | Receptor EMR4 |
| Family 7TM\|B\|Orphan\|CD97 | GPR98 | Receptor very large G protein-coupled receptor 1 |
| Family 7TM\|B\|Orphan\|GPR110/111/113/115/116 | GPR111 | Receptor G-protein coupled receptor 111 |
| Family 7TM\|B\|Orphan\|GPR110/111/113/115/116 | GPR113 | Receptor G-protein coupled receptor 113 |
| Family 7TM\|B\|Orphan\|GPR110/111/113/115/116 | GPR115 | Receptor G-protein coupled receptor 115 |
| Family 7TM\|B\|Orphan\|GPR110/111/113/115/116 | GPR116 | Receptor G-protein coupled receptor 116 |
| Family 7TM\|B\|Orphan\|GPR110/111/113/115/116 | similar to G- | Receptor similar to G-protein coupled receptor 116 |
| | protein coupled | |
| | receptor 116 | |
| Family 7TM\|B\|Orphan\|GPR56/114/64/112 | GPR112 | Receptor G-protein coupled receptor 112 |
| Family 7TM\|B\|Orphan\|GPR56/114/64/112 | GPR114 | Receptor G-protein coupled receptor 114 |
| Family 7TM\|B\|Orphan\|GPR56/114/64/112 | GPR126 | Receptor GPR126 |
| Family 7TM\|B\|Orphan\|GPR56/114/64/112 | GPR56 | Receptor G protein-coupled receptor 56 |
| Family 7TM\|B\|Orphan\|GPR56/114/64/112 | GPR64 | Receptor G protein-coupled receptor 64 |
| Family 7TM\|B\|Polypeptide | ADCYAP1R1 | Receptor type I adenylate cyclase activating polypeptide receptor |
| | | precursor |
| Family 7TM\|B\|Polypeptide | CALCR | Receptor calcitonin receptor |
| Family 7TM\|B\|Polypeptide | CALCRL | Receptor calcitonin receptor-like |
| Family 7TM\|B\|Polypeptide | CRHR1 | Receptor corticotropin releasing hormone receptor 1 |
| Family 7TM\|B\|Polypeptide | CRHR2 | Receptor corticotropin releasing hormone receptor 2 |
| Family 7TM\|B\|Polypeptide | GCGR | Receptor glucagon receptor |
| Family 7TM\|B\|Polypeptide | GHRHR | Receptor growth hormone releasing hormone receptor |
| Family 7TM\|B\|Polypeptide | GIPR | Receptor gastric inhibitory polypeptide receptor |
| Family 7TM\|B\|Polypeptide | GLP1R | Receptor glucagon-like peptide 1 receptor |
| Family 7TM\|B\|Polypeptide | GLP2R | Receptor glucagon-like peptide 2 receptor precursor |
| Family 7TM\|B\|Polypeptide | PTHR1 | Receptor parathyroid hormone receptor 1 |
| Family 7TM\|B\|Polypeptide | PTHR2 | Receptor parathyroid hormone receptor 2 |
| Family 7TM\|B\|Polypeptide | SCTR | Receptor secretin receptor precursor |
| Family 7TM\|B\|Polypeptide | VIPR1 | Receptor vasoactive intestinal peptide receptor 1 |
| Family 7TM\|B\|Polypeptide | VIPR2 | Receptor vasoactive intestinal peptide receptor 2 |
| Family 7TM\|C | GPR156 | Receptor GPR156 |
| Family 7TM\|C | TAS1R1 | Receptor taste receptor, type 1, member 1 |
| Family 7TM\|C | TAS1R2 | Receptor taste receptor, type 1, member 2 |
| Family 7TM\|C | TAS1R3 | Receptor TAS1R3 |
| Family 7TM\|C\|Amine derivatives\|GABA | GABBR1 | Receptor gamma-aminobutyric acid (GABA) B receptor 1 isoform a |
| | | precursor |
| Family 7TM\|C\|Amine derivatives\|GABA | GABBR2 | Receptor G protein-coupled receptor 51 |
| Family 7TM\|C\|Amine derivatives\|Metabotropic | GPRC5B | Receptor G protein-coupled receptor, family C, group 5, member B |
| | | precursor |
| Family 7TM\|C\|Amine derivatives\|Metabotropic | GPRC5C | Receptor G protein-coupled receptor, family C, group 5, member C, |
| | | isoform b, precursor |
| Family 7TM\|C\|Amine derivatives\|Metabotropic | GPRC5D | Receptor G protein-coupled receptor, family C, group 5, member D |
| Family 7TM\|C\|Amine derivatives\|Metabotropic | GPRC6A | Receptor G protein-coupled receptor, family C, group 6, member A |
| Family 7TM\|C\|Amine derivatives\|Metabotropic | GRM1 | Receptor glutamate receptor, metabotropic 1 |
| Family 7TM\|C\|Amine derivatives\|Metabotropic | GRM2 | Receptor glutamate receptor, metabotropic 2 precursor |
| Family 7TM\|C\|Amine derivatives\|Metabotropic | GRM3 | Receptor glutamate receptor, metabotropic 3 precursor |
| Family 7TM\|C\|Amine derivatives\|Metabotropic | GRM4 | Receptor glutamate receptor, metabotropic 4 |
| Family 7TM\|C\|Amine derivatives\|Metabotropic | GRM5 | Receptor glutamate receptor, metabotropic 5 |
| Family 7TM\|C\|Amine derivatives\|Metabotropic | GRM6 | Receptor glutamate receptor, metabotropic 6 precursor |
| Family 7TM\|C\|Amine derivatives\|Metabotropic | GRM7 | Receptor glutamate receptor, metabotropic 7 |
| Family 7TM\|C\|Amine derivatives\|Metabotropic | GRM8 | Receptor glutamate receptor, metabotropic 8 precursor |
| Family 7TM\|C\|Ion\|Calcium | CASR | Receptor calcium-sensing receptor (hypocalciuric hypercalcemia 1, |
| | | severe neonatal hyperparathyroidism) |
| Family 7TM\|D\|Olfactory | OR10A4 | Receptor similar to Olfactory receptor 10A4 (HP2) (Olfactory receptor- |
| | | likeprotein JCG5) |
| Family 7TM\|D\|Olfactory | OR10A5 | Receptor similar to Olfactory receptor 10A5 (HP3) (Olfactory receptor- |
| | | likeprotein JCG6) |
| Family 7TM\|D\|Olfactory | OR10A7 | Receptor similar to seven transmembrane helix receptor |
| Family 7TM\|D\|Olfactory | OR10AD1 | Receptor similar to seven transmembrane helix receptor |
| Family 7TM\|D\|Olfactory | OR10G8 | Receptor similar to seven transmembrane helix receptor |
| Family 7TM\|D\|Olfactory | OR10G9 | Receptor similar to olfactory receptor MOR223-1 |
| Family 7TM\|D\|Olfactory | OR10H1 | Receptor olfactory receptor, family 10, subfamily H, member 1 |
| Family 7TM\|D\|Olfactory | OR10H2 | Receptor olfactory receptor, family 10, subfamily H, member 2 |
| Family 7TM\|D\|Olfactory | OR10H3 | Receptor olfactory receptor, family 10, subfamily H, member 3 |
| Family 7TM\|D\|Olfactory | OR10H4 | Receptor similar to Olfactory receptor 10H3 |
| Family 7TM\|D\|Olfactory | OR10J1 | Receptor olfactory receptor, family 10, subfamily J, member 1 |
| Family 7TM\|D\|Olfactory | OR10J5 | Receptor similar to seven transmembrane helix receptor |
| Family 7TM\|D\|Olfactory | OR10Q1 | Receptor similar to olfactory receptor MOR266-1 |
| Family 7TM\|D\|Olfactory | OR10S1 | Receptor similar to seven transmembrane helix receptor |
| Family 7TM\|D\|Olfactory | OR10T2 | Receptor similar to seven transmembrane helix receptor |
| Family 7TM\|D\|Olfactory | OR10X1 | Receptor similar to olfactory receptor MOR267-7 |
| Family 7TM\|D\|Olfactory | OR10Z1 | Receptor similar to olfactory receptor MOR267-6 |
| Family 7TM\|D\|Olfactory | OR11A1 | Receptor olfactory receptor, family 11, subfamily A, member 1 |
| Family 7TM\|D\|Olfactory | OR11H6 | Receptor similar to seven transmembrane helix receptor |
| Family 7TM\|D\|Olfactory | OR11M1P | Receptor similar to olfactory receptor MOR122-1 |
| Family 7TM\|D\|Olfactory | OR12D2 | Receptor olfactory receptor, family 12, subfamily D, member 2 |
| Family 7TM\|D\|Olfactory | OR12D3 | Receptor olfactory receptor, family 12, subfamily D, member 3 |
| Family 7TM\|D\|Olfactory | OR13C3 | Receptor similar to seven transmembrane helix receptor |
| Family 7TM\|D\|Olfactory | OR13C4 | Receptor similar to seven transmembrane helix receptor |
| Family 7TM\|D\|Olfactory | OR13C5 | Receptor similar to seven transmembrane helix receptor |
| Family 7TM\|D\|Olfactory | OR13C9 | Receptor similar to seven transmembrane helix receptor |
| Family 7TM\|D\|Olfactory | OR1A1 | Receptor olfactory receptor, family 1, subfamily A, member 1 |
| Family 7TM\|D\|Olfactory | OR1A2 | Receptor olfactory receptor, family 1, subfamily A, member 2 |
| Family 7TM\|D\|Olfactory | OR1D2 | Receptor olfactory receptor, family 1, subfamily D, member 2 |
| Family 7TM\|D\|Olfactory | OR1D4 | Receptor olfactory receptor, family 1, subfamily D, member 4 |
| Family 7TM\|D\|Olfactory | OR1E1 | Receptor olfactory receptor, family 1, subfamily E, member 1 |
| Family 7TM\|D\|Olfactory | OR1E2 | Receptor olfactory receptor, family 1, subfamily E, member 2 |
| Family 7TM\|D\|Olfactory | OR1F1 | Receptor olfactory receptor, family 1, subfamily F, member 1 |
| Family 7TM\|D\|Olfactory | OR1F2 | Receptor similar to olfactory receptor, family 1, subfamily F, member 1 |
| Family 7TM\|D\|Olfactory | OR1G1 | Receptor olfactory receptor, family 1, subfamily 6, member 1 |
| Family 7TM\|D\|Olfactory | OR1I1 | Receptor similar to Olfactory receptor 1I1 (Olfactory receptor 19-20) |
| | | (OR19-20) |
| Family 7TM\|D\|Olfactory | OR1L8 | Receptor similar to olfactory receptor MOR138-2 |
| Family 7TM\|D\|Olfactory | OR1M1 | Receptor similar to olfactory receptor MOR132-1 |
| Family 7TM\|D\|Olfactory | OR1S1 | Receptor similar to seven transmembrane helix receptor |
| Family 7TM\|D\|Olfactory | OR1S2 | Receptor similar to olfactory receptor MOR127-1 |
| Family 7TM\|D\|Olfactory | OR2A15P | Receptor similar to olfactory receptor MOR261-4 |
| Family 7TM\|D\|Olfactory | OR2A4 | Receptor olfactory receptor, family 2, subfamily A, member 4 |
| Family 7TM\|D\|Olfactory | OR2AG1 | Receptor similar to Olfactory receptor 2AG1 (HT3) |
| Family 7TM\|D\|Olfactory | OR2AI1P | Receptor similar to seven transmembrane helix receptor |
| Family 7TM\|D\|Olfactory | OR2AP1 | Receptor similar to olfactory receptor MOR115-1 |
| Family 7TM\|D\|Olfactory | OR2B11 | Receptor similar to olfactory receptor MOR256-14 |
| Family 7TM\|D\|Olfactory | OR2B2 | Receptor olfactory receptor, family 2, subfamily B, member 2 |
| Family 7TM\|D\|Olfactory | OR2C1 | Receptor olfactory receptor, family 2, subfamily C, member 1 |
| Family 7TM\|D\|Olfactory | OR2C3 | Receptor similar to seven transmembrane helix receptor |
| Family 7TM\|D\|Olfactory | OR2D2 | Receptor similar to Olfactory receptor 2D2 (Olfactory receptor 11-610) |
| | | 610)(OR11-610) (HB2) |
| Family 7TM\|D\|Olfactory | OR2D3 | Receptor similar to B5 olfactory receptor |
| Family 7TM\|D\|Olfactory | OR2F1 | Receptor olfactory receptor, family 2, subfamily F, member 1 |
| Family 7TM\|D\|Olfactory | OR2H1 | Receptor similar to Olfactory receptor 2H1 (Hs6M1-16) (Olfactory |
| | | receptor6-2) (OR6-2) (OLFR42A-9004 |
| Family 7TM\|D\|Olfactory | OR2H2 | Receptor olfactory receptor, family 2, subfamily H, member 3 |
| Family 7TM\|D\|Olfactory | OR2M5 | Receptor similar to seven transmembrane helix receptor |
| Family 7TM\|D\|Olfactory | OR2S2 | Receptor olfactory receptor, family 2, subfamily S, member 2 |
| Family 7TM\|D\|Olfactory | OR2T10 | Receptor similar to olfactory receptor MOR275-2 |
| Family 7TM\|D\|Olfactory | OR2T12 | Receptor similar to seven transmembrane helix receptor |
| Family 7TM\|D\|Olfactory | OR2T34 | Receptor similar to olfactory receptor MOR275-1 |
| Family 7TM\|D\|Olfactory | OR2T4 | Receptor similar to seven transmembrane helix receptor |
| Family 7TM\|D\|Olfactory | OR2W1 | Receptor olfactory receptor, family 2, subfamily W, member 1 |
| Family 7TM\|D\|Olfactory | OR2Y1 | Receptor similar to seven transmembrane helix receptor |
| Family 7TM\|D\|Olfactory | OR3A1 | Receptor olfactory receptor, family 3, subfamily A, member 1 |
| Family 7TM\|D\|Olfactory | OR3A3 | Receptor olfactory receptor, family 3, subfamily A, member 3 |
| Family 7TM\|D\|Olfactory | OR4C12 | Receptor similar to olfactory receptor MOR232-9 |
| Family 7TM\|D\|Olfactory | OR4C50P | Receptor similar to seven transmembrane helix receptor |
| Family 7TM\|D\|Olfactory | OR4D2 | Receptor similar to Olfactory receptor 4D2 |
| Family 7TM\|D\|Olfactory | OR4F29 | Receptor similar to Olfactory receptor 4F3 |
| Family 7TM\|D\|Olfactory | OR4K14 | Receptor similar to seven transmembrane helix receptor |
| Family 7TM\|D\|Olfactory | OR4L1 | Receptor similar to olfactory receptor MOR247-2 |
| Family 7TM\|D\|Olfactory | OR4S1 | Receptor similar to olfactory receptor MOR226-1 |
| Family 7TM\|D\|Olfactory | OR4S2 | Receptor similar to seven transmembrane helix receptor |
| Family 7TM\|D\|Olfactory | OR4X2 | Receptor similar to olfactory receptor MOR228-3 |
| Family 7TM\|D\|Olfactory | OR51A7 | Receptor similar to seven transmembrane helix receptor |
| Family 7TM\|D\|Olfactory | OR51B2 | Receptor olfactory receptor, family 51, subfamily B, member 2 |
| Family 7TM\|D\|Olfactory | OR51B4 | Receptor olfactory receptor, family 51, subfamily B, member 4 |
| Family 7TM\|D\|Olfactory | OR51E2 | Receptor prostate specific G-protein coupled receptor |
| Family 7TM\|D\|Olfactory | OR51F1 | Receptor similar to olfactory receptor |
| Family 7TM\|D\|Olfactory | OR51L1 | Receptor similar to olfactory receptor MOR7-1 |
| Family 7TM\|D\|Olfactory | OR51N1P | Receptor similar to seven transmembrane helix receptor |
| Family 7TM\|D\|Olfactory | OR51S1 | Receptor similar to olfactory receptor MOR21-1 |
| Family 7TM\|D\|Olfactory | OR52A1 | Receptor olfactory receptor, family 52, subfamily A, member 1 |
| Family 7TM\|D\|Olfactory | OR52B2 | Receptor similar to Olfactory receptor 52B2 |
| Family 7TM\|D\|Olfactory | OR52P2P | Receptor similar to seven transmembrane helix receptor |
| Family 7TM\|D\|Olfactory | OR52R1 | Receptor similar to seven transmembrane helix receptor |
| Family 7TM\|D\|Olfactory | OR52W1 | Receptor similar to olfactory receptor MOR36-1 |
| Family 7TM\|D\|Olfactory | OR52Z1P | Receptor similar to odorant receptor HOR3beta2 |
| Family 7TM\|D\|Olfactory | OR56B4 | Receptor similar to olfactory receptor MOR40-3 |
| Family 7TM\|D\|Olfactory | OR5A2 | Receptor similar to seven transmembrane helix receptor |
| Family 7TM\|D\|Olfactory | OR5AR1 | Receptor similar to olfactory receptor MOR180-1 |
| Family 7TM\|D\|Olfactory | OR5AT1 | Receptor similar to seven transmembrane helix receptor |
| Family 7TM\|D\|Olfactory | OR5AV1P | Receptor similar to seven transmembrane helix receptor |
| Family 7TM\|D\|Olfactory | OR5B21 | Receptor similar to olfactory receptor MOR202-4 |
| Family 7TM\|D\|Olfactory | OR5BF1 | Receptor similar to olfactory receptor MOR220-2 |
| Family 7TM\|D\|Olfactory | OR5D18 | Receptor similar to odorant receptor |
| Family 7TM\|D\|Olfactory | OR5I1 | Receptor olfactory receptor, family 5, subfamily I, member 1 |
| Family 7TM\|D\|Olfactory | OR5L1 | Receptor similar to olfactory receptor MOR174-1 |
| Family 7TM\|D\|Olfactory | OR5M3 | Receptor similar to seven transmembrane helix receptor |
| Family 7TM\|D\|Olfactory | OR5M8 | Receptor similar to seven transmembrane helix receptor |
| Family 7TM\|D\|Olfactory | OR5P2 | Receptor olfactory receptor-like protein JCG3 |
| Family 7TM\|D\|Olfactory | OR5P3 | Receptor olfactory receptor-like protein JCG1 |
| Family 7TM\|D\|Olfactory | OR5R1 | Receptor similar to olfactory receptor MOR185-3 |
| Family 7TM\|D\|Olfactory | OR5T2 | Receptor similar to seven transmembrane helix receptor |
| Family 7TM\|D\|Olfactory | OR5V1 | Receptor olfactory receptor, family 5, subfamily V, member 1 |
| Family 7TM\|D\|Olfactory | OR6A2 | Receptor olfactory receptor, family 6, subfamily A, member 1 |
| Family 7TM\|D\|Olfactory | OR6C6 | Receptor similar to olfactory receptor MOR110-6 |
| Family 7TM\|D\|Olfactory | OR6K4P | Receptor similar to seven transmembrane helix receptor |
| Family 7TM\|D\|Olfactory | OR6K6 | Receptor similar to olfactory receptor MOR105-4 |
| Family 7TM\|D\|Olfactory | OR6N1 | Receptor similar to olfactory receptor MOR105-1 |
| Family 7TM\|D\|Olfactory | OR6P1 | Receptor similar to seven transmembrane helix receptor |
| Family 7TM\|D\|Olfactory | OR6Q1 | Receptor similar to seven transmembrane helix receptor |
| Family 7TM\|D\|Olfactory | OR6T1 | Receptor similar to olfactory receptor C6 |
| Family 7TM\|D\|Olfactory | OR6W1P | Receptor olfactory receptor sdolf |
| Family 7TM\|D\|Olfactory | OR7A17 | Receptor olfactory receptor, family 7, subfamily A, member 17 |
| Family 7TM\|D\|Olfactory | OR7A5 | Receptor olfactory receptor, family 7, subfamily C, member 1 |
| Family 7TM\|D\|Olfactory | OR7C2 | Receptor olfactory receptor, family 7, subfamily C, member 3 |
| Family 7TM\|D\|Olfactory | OR7D2 | Receptor similar to Olfactory receptor 7A10 (OST027) |
| Family 7TM\|D\|Olfactory | OR7D4 | Receptor similar to seven transmembrane helix receptor |
| Family 7TM\|D\|Olfactory | OR7E5P | Receptor seven transmembrane helix receptor |
| Family 7TM\|D\|Olfactory | OR8B12 | Receptor similar to olfactory receptor MOR161-4 |
| Family 7TM\|D\|Olfactory | OR8B4 | Receptor similar to Olfactory receptor 8B4 |
| Family 7TM\|D\|Olfactory | OR8B8 | Receptor similar to Olfactory receptor 8B8 (Olfactory receptor |
| | | TPCR85)(Olfactory-like receptor JCG8) |
| Family 7TM\|D\|Olfactory | OR8H1 | Receptor similar to olfactory receptor MOR206-3 |
| Family 7TM\|D\|Olfactory | OR8I2 | Receptor similar to olfactory receptor MOR207-1 |
| Family 7TM\|D\|Olfactory | OR8K3 | Receptor similar to olfactory receptor MOR188-3 |
| Family 7TM\|D\|Olfactory | OR8K5 | Receptor similar to olfactory receptor MOR185-2 |
| Family 7TM\|D\|Olfactory | OR9G4 | Receptor similar to olfactory receptor MOR213-4 |
| Family 7TM\|D\|Olfactory | OR9I1 | Receptor similar to olfactory receptor MOR211-1 |
| Family 7TM\|D\|Olfactory | OR9K1P | Receptor similar to seven transmembrane helix receptor |
| Receptor Family | Gene | Receptor |
| Family 7TM\|D\|Olfactory | similar to B6 olfactory receptor | Receptor similar to B6 olfactory receptor |
| Family 7TM\|D\|Olfactory | similar to odorant receptor HOR3beta1 | Receptor similar to odorant receptor HOR3beta1 |
| Family 7TM\|D\|Olfactory | similar to odorant receptor MOR10 | Receptor similar to odorant receptor MOR10 |
| Family 7TM\|D\|Olfactory | similar to OL1 receptor | Receptor similar to OL1 receptor |
| Family 7TM\|D\|Olfactory | similar to olfactory receptor | Receptor similar to olfactory receptor |
| Family 7TM\|D\|Olfactory | similar to olfactory receptor | Receptor similar to olfactory receptor |
| Family 7TM\|D\|Olfactory | similar to olfactory receptor | Receptor similar to olfactory receptor |
| Family 7TM\|D\|Olfactory | similar to olfactory receptor | Receptor similar to olfactory receptor |
| Family 7TM\|D\|Olfactory | similar to olfactory receptor | Receptor similar to olfactory receptor |
| Family 7TM\|D\|Olfactory | similar to olfactory receptor | Receptor similar to olfactory receptor |
| Family 7TM\|D\|Olfactory | similar to olfactory receptor 17-210 | Receptor similar to olfactory receptor 17-210 |
| Family 7TM\|D\|Olfactory | similar to Olfactory receptor 2B3 (Olfactory | Receptor similar to Olfactory receptor 2B3 (Olfactory receptor 6-4) |
| | receptor 6-4) (OR6-4)(Hs6M1-1) | (OR6-4)(Hs6M1-1) |
| Family 7TM\|D\|Olfactory | similar to Olfactory receptor 2F2 (Olfactory | Receptor similar to Olfactory receptor 2F2 (Olfactory receptor 7-1) |
| | receptor 7-1) (OR7-1) | (OR7-1) |
| Family 7TM\|D\|Olfactory | similar to Olfactory receptor 2J3 (Olfactory | Receptor similar to Olfactory receptor 2J3 (Olfactory receptor 6-6) (OR6- |
| | receptor 6-6) (OR6-6)(Hs6M1-3) | 6)(Hs6M1-3) |
| Family 7TM\|D\|Olfactory | similar to Olfactory receptor 2T1 (Olfactory | Receptor similar to Olfactory receptor 2T1 (Olfactory receptor 1- |
| | receptor 1-25)(OR1-25) | 25)(OR1-25) |
| Family 7TM\|D\|Olfactory | similar to Olfactory receptor 2T1 (Olfactory | Receptor similar to Olfactory receptor 2T1 (Olfactory receptor 1- |
| | receptor 1-25)(OR1-25) | 25)(OR1-25) |
| Family 7TM\|D\|Olfactory | similar to olfactory receptor 37a | Receptor similar to olfactory receptor 37a |
| Family 7TM\|D\|Olfactory | similar to Olfactory receptor 4F3 | Receptor similar to Olfactory receptor 4F3 |
| Family 7TM\|D\|Olfactory | similar to Olfactory receptor 52E6 | Receptor similar to Olfactory receptor 52E6 |
| Family 7TM\|D\|Olfactory | similar to Olfactory receptor 52E6 | Receptor similar to Olfactory receptor 52E6 |
| Family 7TM\|D\|Olfactory | similar to Olfactory receptor 5B16 | Receptor similar to Olfactory receptor 5B16 |
| Family 7TM\|D\|Olfactory | similar to Olfactory receptor 5F1 (Olfactory | Receptor similar to Olfactory receptor 5F1 (Olfactory receptor 11- |
| | receptor 11-10)(OR11-10) | 10)(OR11-10) |
| Family 7TM\|D\|Olfactory | similar to Olfactory receptor 5F1 (Olfactory | Receptor similar to Olfactory receptor 5F1 (Olfactory receptor 11- |
| | receptor 11-10)(OR11-10) | 10)(OR11-10) |
| Family 7TM\|D\|Olfactory | similar to Olfactory receptor 5U1 (Hs6M1-28) | Receptor similar to Olfactory receptor 5U1 (Hs6M1-28) |
| Family 7TM\|D\|Olfactory | similar to Olfactory receptor 6B1 (Olfactory | Receptor similar to Olfactory receptor 6B1 (Olfactory receptor 7-3) |
| | receptor 7-3) (OR7-3) | (OR7-3) |
| Family 7TM\|D\|Olfactory | similar to olfactory receptor 71 | Receptor similar to olfactory receptor 71 |
| Family 7TM\|D\|Olfactory | similar to Olfactory receptor 7A10 (OST027) | Receptor similar to Olfactory receptor 7A10 (OST027) |
| Family 7TM\|D\|Olfactory | similar to Olfactory receptor 8D1 (Olfactory | Receptor similar to Olfactory receptor 8D1 (Olfactory receptor-like |
| | receptor-like proteinJCG9) (OST004) | proteinJCG9) (OST004) |
| Family 7TM\|D\|Olfactory | similar to Olfactory receptor 8D2 (Olfactory | Receptor similar to Olfactory receptor 8D2 (Olfactory receptor-like |
| | receptor-like proteinJCG2) | proteinJCG2) |
| Family 7TM\|D\|Olfactory | similar to olfactory receptor MOR10-1 | Receptor similar to olfactory receptor MOR10-1 |
| Family 7TM\|D\|Olfactory | similar to olfactory receptor MOR10-1 | Receptor similar to olfactory receptor MOR10-1 |
| Family 7TM\|D\|Olfactory | similar to olfactory receptor MOR103-10 | Receptor similar to olfactory receptor MOR103-10 |
| Family 7TM\|D\|Olfactory | similar to olfactory receptor MOR104-3 | Receptor similar to olfactory receptor MOR104-3 |
| Family 7TM\|D\|Olfactory | similar to olfactory receptor MOR105-5P | Receptor similar to olfactory receptor MOR105-5P |
| Family 7TM\|D\|Olfactory | similar to olfactory receptor MOR106-12 | Receptor similar to olfactory receptor MOR106-12 |
| Family 7TM\|D\|Olfactory | similar to olfactory receptor MOR109-1 | Receptor similar to olfactory receptor MOR109-1 |
| Family 7TM\|D\|Olfactory | similar to olfactory receptor MOR110-6 | Receptor similar to olfactory receptor MOR110-6 |
| Family 7TM\|D\|Olfactory | similar to olfactory receptor MOR111-1 | Receptor similar to olfactory receptor MOR111-1 |
| Family 7TM\|D\|Olfactory | similar to olfactory receptor MOR111-4 | Receptor similar to olfactory receptor MOR111-4 |
| Family 7TM\|D\|Olfactory | similar to olfactory receptor MOR112-1 | Receptor similar to olfactory receptor MOR112-1 |
| Family 7TM\|D\|Olfactory | similar to olfactory receptor MOR112-1 | Receptor similar to olfactory receptor MOR112-1 |
| Family 7TM\|D\|Olfactory | similar to olfactory receptor MOR113-1 | Receptor similar to olfactory receptor MOR113-1 |
| Family 7TM\|D\|Olfactory | similar to olfactory receptor MOR118-1 | Receptor similar to olfactory receptor MOR118-1 |
| Family 7TM\|D\|Olfactory | similar to olfactory receptor MOR120-2 | Receptor similar to olfactory receptor MOR120-2 |
| Family 7TM\|D\|Olfactory | similar to olfactory receptor MOR14-10 | Receptor similar to olfactory receptor MOR14-10 |
| Family 7TM\|D\|Olfactory | similar to olfactory receptor MOR14-2 | Receptor similar to olfactory receptor MOR14-2 |
| Family 7TM\|D\|Olfactory | similar to olfactory receptor MOR14-3 | Receptor similar to olfactory receptor MOR14-3 |
| Family 7TM\|D\|Olfactory | similar to olfactory receptor MOR145-2 | Receptor similar to olfactory receptor MOR145-2 |
| Family 7TM\|D\|Olfactory | similar to olfactory receptor MOR14-9 | Receptor similar to olfactory receptor MOR14-9 |
| Family 7TM\|D\|Olfactory | similar to olfactory receptor MOR16-1 | Receptor similar to olfactory receptor MOR16-1 |
| Family 7TM\|D\|Olfactory | similar to olfactory receptor MOR167-3 | Receptor similar to olfactory receptor MOR167-3 |
| Family 7TM\|D\|Olfactory | similar to olfactory receptor MOR174-10 | Receptor similar to olfactory receptor MOR174-10 |
| Family 7TM\|D\|Olfactory | similar to olfactory receptor MOR176-1 | Receptor similar to olfactory receptor MOR176-1 |
| Family 7TM\|D\|Olfactory | similar to olfactory receptor MOR183-1 | Receptor similar to olfactory receptor MOR183-1 |
| Family 7TM\|D\|Olfactory | similar to olfactory receptor MOR183-1 | Receptor similar to olfactory receptor MOR183-1 |
| Family 7TM\|D\|Olfactory | similar to olfactory receptor MOR183-1 | Receptor similar to olfactory receptor MOR183-1 |
| Family 7TM\|D\|Olfactory | similar to olfactory receptor MOR185-1 | Receptor similar to olfactory receptor MOR185-1 |
| Family 7TM\|D\|Olfactory | similar to olfactory receptor MOR185-2 | Receptor similar to olfactory receptor MOR185-2 |
| Family 7TM\|D\|Olfactory | similar to olfactory receptor MOR185-4 | Receptor similar to olfactory receptor MOR185-4 |
| Family 7TM\|D\|Olfactory | similar to olfactory receptor MOR185-5 | Receptor similar to olfactory receptor MOR185-5 |
| Family 7TM\|D\|Olfactory | similar to olfactory receptor MOR187-1 | Receptor similar to olfactory receptor MOR187-1 |
| Family 7TM\|D\|Olfactory | similar to olfactory receptor MOR194-1 | Receptor similar to olfactory receptor MOR194-1 |
| Family 7TM\|D\|Olfactory | similar to olfactory receptor MOR196-1 | Receptor similar to olfactory receptor MOR196-1 |
| Family 7TM\|D\|Olfactory | similar to olfactory receptor MOR196-2 | Receptor similar to olfactory receptor MOR196-2 |
| Family 7TM\|D\|Olfactory | similar to olfactory receptor MOR196-3 | Receptor similar to olfactory receptor MOR196-3 |
| Family 7TM\|D\|Olfactory | similar to olfactory receptor MOR196-4 | Receptor similar to olfactory receptor MOR196-4 |
| Family 7TM\|D\|Olfactory | similar to olfactory receptor MOR196-4 | Receptor similar to olfactory receptor MOR196-4 |
| Family 7TM\|D\|Olfactory | similar to olfactory receptor MOR199-1 | Receptor similar to olfactory receptor MOR199-1 |
| Family 7TM\|D\|Olfactory | similar to olfactory receptor MOR201-2 | Receptor similar to olfactory receptor MOR201-2 |
| Family 7TM\|D\|Olfactory | similar to olfactory receptor MOR203-2 | Receptor similar to olfactory receptor MOR203-2 |
| Family 7TM\|D\|Olfactory | similar to olfactory receptor MOR203-3 | Receptor similar to olfactory receptor MOR203-3 |
| Family 7TM\|D\|Olfactory | similar to olfactory receptor MOR204-2 | Receptor similar to olfactory receptor MOR204-2 |
| Family 7TM\|D\|Olfactory | similar to olfactory receptor MOR205-1 | Receptor similar to olfactory receptor MOR205-1 |
| Family 7TM\|D\|Olfactory | similar to olfactory receptor MOR206-3 | Receptor similar to olfactory receptor MOR206-3 |
| Family 7TM\|D\|Olfactory | similar to olfactory receptor MOR206-4 | Receptor similar to olfactory receptor MOR206-4 |
| Family 7TM\|D\|Olfactory | similar to olfactory receptor MOR209-1 | Receptor similar to olfactory receptor MOR209-1 |
| Family 7TM\|D\|Olfactory | similar to olfactory receptor MOR210-1 | Receptor similar to olfactory receptor MOR210-1 |
| Family 7TM\|D\|Olfactory | similar to olfactory receptor MOR212-1 | Receptor similar to olfactory receptor MOR212-1 |
| Family 7TM\|D\|Olfactory | similar to olfactory receptor MOR212-1 | Receptor similar to olfactory receptor MOR212-1 |
| Family 7TM\|D\|Olfactory | similar to olfactory receptor MOR212-3 | Receptor similar to olfactory receptor MOR212-3 |
| Family 7TM\|D\|Olfactory | similar to olfactory receptor MOR213-6 | Receptor similar to olfactory receptor MOR213-6 |
| Family 7TM\|D\|Olfactory | similar to olfactory receptor MOR214-4 | Receptor similar to olfactory receptor MOR214-4 |
| Family 7TM\|D\|Olfactory | similar to olfactory receptor MOR215-1 | Receptor similar to olfactory receptor MOR215-1 |
| Family 7TM\|D\|Olfactory | similar to olfactory receptor MOR215-3 | Receptor similar to olfactory receptor MOR215-3 |
| Family 7TM\|D\|Olfactory | similar to olfactory receptor MOR223-1 | Receptor similar to olfactory receptor MOR223-1 |
| Family 7TM\|D\|Olfactory | similar to olfactory receptor MOR223-3 | Receptor similar to olfactory receptor MOR223-3 |
| Family 7TM\|D\|Olfactory | similar to olfactory receptor MOR227-1 | Receptor similar to olfactory receptor MOR227-1 |
| Family 7TM\|D\|Olfactory | similar to olfactory receptor MOR232-3 | Receptor similar to olfactory receptor MOR232-3 |
| Family 7TM\|D\|Olfactory | similar to olfactory receptor MOR232-3 | Receptor similar to olfactory receptor MOR232-3 |
| Family 7TM\|D\|Olfactory | similar to olfactory receptor MOR233-18 | Receptor similar to olfactory receptor MOR233-18 |
| Family 7TM\|D\|Olfactory | similar to olfactory receptor MOR237-2 | Receptor similar to olfactory receptor MOR237-2 |
| Family 7TM\|D\|Olfactory | similar to olfactory receptor MOR239-1 | Receptor similar to olfactory receptor MOR239-1 |
| Family 7TM\|D\|Olfactory | similar to olfactory receptor MOR239-1 | Receptor similar to olfactory receptor MOR239-1 |
| Family 7TM\|D\|Olfactory | similar to olfactory receptor MOR239-2 | Receptor similar to olfactory receptor MOR239-2 |
| Family 7TM\|D\|Olfactory | similar to olfactory receptor MOR239-3 | Receptor similar to olfactory receptor MOR239-3 |
| Family 7TM\|D\|Olfactory | similar to olfactory receptor MOR239-5 | Receptor similar to olfactory receptor MOR239-5 |
| Family 7TM\|D\|Olfactory | similar to olfactory receptor MOR239-6 | Receptor similar to olfactory receptor MOR239-6 |
| Family 7TM\|D\|Olfactory | similar to olfactory receptor MOR241-3 | Receptor similar to olfactory receptor MOR241-3 |
| Family 7TM\|D\|Olfactory | similar to olfactory receptor MOR245-1 | Receptor similar to olfactory receptor MOR245-1 |
| Family 7TM\|D\|Olfactory | similar to olfactory receptor MOR245-3 | Receptor similar to olfactory receptor MOR245-3 |
| Family 7TM\|D\|Olfactory | similar to olfactory receptor MOR245-5 | Receptor similar to olfactory receptor MOR245-5 |
| Family 7TM\|D\|Olfactory | similar to olfactory receptor MOR245-8 | Receptor similar to olfactory receptor MOR245-8 |
| Family 7TM\|D\|Olfactory | similar to olfactory receptor MOR246-2 | Receptor similar to olfactory receptor MOR246-2 |
| Family 7TM\|D\|Olfactory | similar to olfactory receptor MOR246-2 | Receptor similar to olfactory receptor MOR246-2 |
| Family 7TM\|D\|Olfactory | similar to olfactory receptor MOR246-2 | Receptor similar to olfactory receptor MOR246-2 |
| Family 7TM\|D\|Olfactory | similar to olfactory receptor MOR247-1 | Receptor similar to olfactory receptor MOR247-1 |
| Family 7TM\|D\|Olfactory | similar to olfactory receptor MOR25-1 | Receptor similar to olfactory receptor MOR25-1 |
| Family 7TM\|D\|Olfactory | similar to olfactory receptor MOR256-12 | Receptor similar to olfactory receptor MOR256-12 |
| Family 7TM\|D\|Olfactory | similar to olfactory receptor MOR256-12 | Receptor similar to olfactory receptor MOR256-12 |
| Family 7TM\|D\|Olfactory | similar to olfactory receptor MOR256-3 | Receptor similar to olfactory receptor MOR256-3 |
| Family 7TM\|D\|Olfactory | similar to olfactory receptor MOR256-8 | Receptor similar to olfactory receptor MOR256-8 |
| Family 7TM\|D\|Olfactory | similar to olfactory receptor MOR261-11 | Receptor similar to olfactory receptor MOR261-11 |
| Family 7TM\|D\|Olfactory | similar to olfactory receptor MOR261-11 | Receptor similar to olfactory receptor MOR261-11 |
| Family 7TM\|D\|Olfactory | similar to olfactory receptor MOR261-13 | Receptor similar to olfactory receptor MOR261-13 |
| Family 7TM\|D\|Olfactory | similar to olfactory receptor MOR261-4 | Receptor similar to olfactory receptor MOR261-4 |
| Family 7TM\|D\|Olfactory | similar to olfactory receptor MOR262-2 | Receptor similar to olfactory receptor MOR262-2 |
| Family 7TM\|D\|Olfactory | similar to olfactory receptor MOR262-7 | Receptor similar to olfactory receptor MOR262-7 |
| Family 7TM\|D\|Olfactory | similar to olfactory receptor MOR265-1 | Receptor similar to olfactory receptor MOR265-1 |
| Family 7TM\|D\|Olfactory | similar to olfactory receptor MOR266-3 | Receptor similar to olfactory receptor MOR266-3 |
| Family 7TM\|D\|Olfactory | similar to olfactory receptor MOR266-4 | Receptor similar to olfactory receptor MOR266-4 |
| Family 7TM\|D\|Olfactory | similar to olfactory receptor MOR267-16 | Receptor similar to olfactory receptor MOR267-16 |
| Family 7TM\|D\|Olfactory | similar to olfactory receptor MOR267-3 | Receptor similar to olfactory receptor MOR267-3 |
| Family 7TM\|D\|Olfactory | similar to olfactory receptor MOR267-8 | Receptor similar to olfactory receptor MOR267-8 |
| Family 7TM\|D\|Olfactory | similar to olfactory receptor MOR270-1 | Receptor similar to olfactory receptor MOR270-1 |
| Family 7TM\|D\|Olfactory | similar to olfactory receptor MOR276-2 | Receptor similar to olfactory receptor MOR276-2 |
| Family 7TM\|D\|Olfactory | similar to olfactory receptor MOR28-1 | Receptor similar to olfactory receptor MOR28-1 |
| Family 7TM\|D\|Olfactory | similar to olfactory receptor MOR282-1 | Receptor similar to olfactory receptor MOR282-1 |
| Family 7TM\|D\|Olfactory | similar to olfactory receptor MOR31-7 | Receptor similar to olfactory receptor MOR31-7 |
| Family 7TM\|D\|Olfactory | similar to olfactory receptor MOR32-3 | Receptor similar to olfactory receptor MOR32-3 |
| Family 7TM\|D\|Olfactory | similar to olfactory receptor MOR32-5 | Receptor similar to olfactory receptor MOR32-5 |
| Family 7TM\|D\|Olfactory | similar to olfactory receptor MOR34-1 | Receptor similar to olfactory receptor MOR34-1 |
| Family 7TM\|D\|Olfactory | similar to olfactory receptor MOR34-1 | Receptor similar to olfactory receptor MOR34-1 |
| Family 7TM\|D\|Olfactory | similar to olfactory receptor MOR8-1 | Receptor similar to olfactory receptor MOR8-1 |
| Family 7TM\|D\|Olfactory | similar to olfactory receptor, family 2, | Receptor similar to olfactory receptor, family 2, subfamily J, member 2 |
| | subfamily J, member 2 | |
| Family 7TM\|D\|Olfactory | similar to olfactory receptor, family 7, | Receptor similar to olfactory receptor, family 7, subfamily A, member 17 |
| | subfamily A, member 17 | |
| Family 7TM\|D\|Olfactory | similar to seven transmembrane helix receptor | Receptor similar to seven transmembrane helix receptor |
| Family 7TM\|Other7TM | GPR107 | Receptor G protein-coupled receptor 107 |
| Family 7TM\|Other7TM | GPR108 | Receptor G protein-coupled receptor 108 |
| Family 7TM\|Other7TM | GPR155 | Receptor G protein-coupled receptor 155 |
| Family 7TM\|Other7TM | GPR75 | Receptor GPR75 |
| Family 7TM\|Other7TM | GPR89A | Receptor G protein-coupled receptor 89 |
| Family 7TM\|Other7TM\|MPR-ADQ | ADIPOR1 | Receptor Adiponectin receptor protein 1 |
| Family 7TM\|Other7TM\|MPR-ADQ | ADIPOR2 | Receptor Adiponectin receptor protein 2 |
| Family 7TM\|Other7TM\|MPR-ADQ | PAQR3 | Receptor progestin and adipoQ receptor family member III |
| Family 7TM\|Other7TM\|MPR-ADQ | PAQR5 | Receptor progestin and adipoQ receptor family member V |
| Family 7TM\|Other7TM\|MPR-ADQ | PAQR6 | Receptor progestin and adipoQ receptor family member VI |
| Family 7TM\|Other7TM\|MPR-ADQ | PAQR7 | Receptor membrane progestin receptor alpha |
| Family 7TM\|Other7TM\|MPR-ADQ | PAQR9 | Receptor progestin and adipoQ receptor family member IX |
| Family 7TM\|Other7TM\|MPR-ADQ | putative membrane steroid receptor | Receptor putative membrane steroid receptor |
| Family Cytokine1R | CSF1R | Receptor Macrophage colony stimulating factor I receptor precursor |
| Family Cytokine1R | EPOR | Receptor Erythropoietin receptor precursor |
| Family Cytokine1R | IL12RB1 | Receptor Interleukin-12 receptor beta-1 chain |
| Family Cytokine1R | IL23R | Receptor interleukin 23 receptor |
| Family Cytokine1R | IL7R | Receptor Interleukin 7 receptor |
| Family Cytokine1R | MPL | Receptor myeloproliferative leukemia virus oncogene |
| Family Cytokine1R\|CICYTR | CNTFR | Receptor ciliary neurotrophic factor receptor alpha precursor |
| Family Cytokine1R\|CICYTR | CRLF1 | Receptor class I cytokine receptor |
| Family Cytokine1R\|CICYTR | IL11RA | Receptor Interleukin 11 receptor, alpha isoform 2 precursor |
| Family Cytokine1R\|CICYTR | IL6R | Receptor interleukin-6 receptor precursor |
| Family Cytokine1R\|GHR | CSF2RB | Receptor Cytokine receptor common beta chain precursor |
| Family Cytokine1R\|GHR | GHR | Receptor Growth hormone receptor [Precursor] |
| Family Cytokine1R\|GHR | LEPR | Receptor Leptin receptor |
| Family Cytokine1R\|IL-1RL2 | IL18R1 | Receptor Interleukin 18 receptor 1 precursor |
| Family Cytokine1R\|IL-1RL2 | IL18RAP | Receptor Interleukin-18 receptor accessory protein-like |
| Family Cytokine1R\|IL-1RL2 | IL1R1 | Receptor Interleukin-1 receptor, type I precursor |
| Family Cytokine1R\|IL-1RL2 | IL1R2 | Receptor Interleukin 1 receptor, type II precursor |
| Family Cytokine1R\|IL-1RL2 | IL1RAP | Receptor Interleukin 1 receptor accessory protein isoform 1 |
| Family Cytokine1R\|IL-1RL2 | IL1RAPL1 | Receptor interleukin 1 receptor accessory protein-like 1 |
| Family Cytokine1R\|IL-1RL2 | IL1RAPL2 | Receptor X-linked interleukin-1 receptor accessory protein-like 2 |
| Family Cytokine1R\|IL-1RL2 | IL1RL1 | Receptor Interleukin 1 receptor-like 1 isoform 1 precursor |
| Family Cytokine1R\|IL-1RL2 | IL1RL1 | Receptor Interleukin 1 receptor-like 1 isoform 2 precursor |
| Family Cytokine1R\|IL-1RL2 | IL1RL1 | Receptor interleukin 1 receptor-like 1 isoform 3 precursor |
| Family Cytokine1R\|IL-21R | IL21R | Receptor interleukin 21 receptor |
| Family Cytokine1R\|IL-21R | IL2RB | Receptor interleukin 2 receptor, beta |
| Family Cytokine1R\|IL-21R | IL9R | Receptor Interleukin-9 receptor [Precursor] |
| Family Cytokine1R\|IL2RA | IL15RA | Receptor interleukin 15 receptor, alpha |
| Family Cytokine1R\|IL2RA | IL2RA | Receptor interleukin 2 receptor, alpha |
| Family Cytokine1R\|IL2RG | CSF2RA | Receptor Granulocyte-macrophage colony-stimulating factor receptor |
| | | alpha chain |
| Family Cytokine1R\|IL2RG | IL13RA1 | Receptor Interleukin-13 receptor alpha-1 chain precursor |
| Family Cytokine1R\|IL2RG | IL13RA2 | Receptor Interleukin-13 receptor alpha-2 chain precursor |
| Family Cytokine1R\|IL2RG | IL2RG | Receptor interleukin 2 receptor, gamma (severe combined |
| | | immunodeficiency) |
| Family Cytokine1R\|IL2RG | IL3RA | Receptor A40266 interleukin-3 receptor alpha chain precursor |
| Family Cytokine1R\|IL2RG | IL5RA | Receptor Interleukin-5 receptor alpha chain [Precursor] |
| Family Cytokine1R\|OSMR | CSF3R | Receptor colony stimulating factor 3 receptor isoform a precursor |
| Family Cytokine1R\|OSMR | IL12RB2 | Receptor Interleukin-12 receptor beta-2 chain precursor |
| Family Cytokine1R\|OSMR | IL31RA | Receptor gp130-like monocyte receptor |
| Family Cytokine1R\|OSMR | IL6ST | Receptor membrane glycoprotein gp130 precursor |
| Family Cytokine1R\|OSMR | LIFR | Receptor Leukemia inhibitory factor receptor precursor |
| Family Cytokine1R\|OSMR | OSMR | Receptor Oncostatin-M specific receptor beta subunit |
| Family Cytokine1R\|PRLR | PRLR | Receptor prolactin receptor long form precursor, hepatoma and breast |
| | | cancer cells |
| Family Cytokine1R\|PRLR | prolactin receptor short form S1a precursor, | Receptor prolactin receptor short form S1a precursor, breast cancer cells |
| | breast cancer cells T-47D | T-47D |
| Family Cytokine2R | IFNAR1 | Receptor Interferon-alpha/beta receptor alpha chain precursor |
| Family Cytokine2R | IFNAR2 | Receptor Interferon-alpha/beta receptor beta chain precursor |
| Family Cytokine2R | IFNGR1 | Receptor Interferon-gamma receptor alpha chain [Precursor] |
| Family Cytokine2R | IFNGR2 | Receptor Interferon-gamma receptor beta chain precursor |
| Family Cytokine2R | IL10RA | Receptor Interleukin-10 receptor alpha chain precursor |
| Family Cytokine2R | IL10RB | Receptor Interleukin-10 receptor beta chain precursor |
| Family Cytokine2R | IL20RA | Receptor Interleukin 20 receptor alpha, isoform 1 |
| Family Cytokine2R | IL20RB | Receptor interleukin 20 receptor beta |
| Family Cytokine2R | IL22RA1 | Receptor interleukin 22 receptor, alpha 1 |
| Family Cytokine2R | IL22RA2 | Receptor Soluble cytokine class II receptor, long isoform [Precursor] |
| Family Cytokine2R | IL28RA | Receptor Interleukin-28 Receptor |
| Family GPI-anchored | GFRA1 | Receptor GDNF family receptor alpha 1 isoform a preproprotein |
| Family GPI-anchored | GFRA2 | Receptor GDNF family receptor alpha 2 |
| Family GPI-anchored | GFRA3 | Receptor GDNF family receptor alpha 3 |
| Family GPI-anchored | GFRA4 | Receptor GDNF family receptor alpha 4 isoform a precursor |
| Family GuaCyc | GUCY2C | Receptor Heat-stable enterotoxin receptor precursor |
| Family GuaCyc | NPR1 | Receptor Atrial natriuretic peptide receptor A precursor |
| Family GuaCyc | NPR2 | Receptor Atrial natriuretic peptide receptor B precursor |
| Family GuaCyc | NPR3 | Receptor Atrial natriuretic peptide clearance receptor precursor |
| Family GuaCyc | similar to guanylyl cyclase receptor G | Receptor similar to guanylyl cyclase receptor G |
| Family IL-17 Receptors | IL17RA | Receptor interleukin 17 receptor |
| Family IL-17 Receptors | IL17RB | Receptor interleukin 17 receptor B |
| Family IL-17 Receptors | IL17RB | Receptor interleukin 17 receptor B |
| Family IL-17 Receptors | IL17RC | Receptor interleukin 17 receptor C |
| Family Integrins | ITGA2 | Receptor integrin, alpha 2 |
| Family Integrins\|ITAB | ITGA5 | Receptor Integrin alpha-5 precursor |
| Family Integrins\|ITAB | ITGAV | Receptor integrin, alpha V (vitronectin receptor, alpha polypeptide, |
| | | antigen CD51) |
| Family Integrins\|ITAM | ITGAM | Receptor integrin, alpha M |
| Family Integrins\|ITB-4 | ITGB1 | Receptor Integrin beta-1 precursor |
| Family Integrins\|ITB-4 | ITGB3 | Receptor integrin beta chain, beta 3 precursor |
| Family LDLLRP | LDLR | Receptor low-density lipoprotein receptor precursor |
| Family LDLLRP | LRP1 | Receptor Low-density lipoprotein receptor-related protein 1 precursor |
| Family LDLLRP | LRP10 | Receptor low density lipoprotein receptor-related protein 10 |
| Family LDLLRP | LRP11 | Receptor low density lipoprotein receptor-related protein 11 |
| Family LDLLRP | LRP2 | Receptor Low-density lipoprotein receptor-related protein 2 precursor |
| Family LDLLRP | LRP3 | Receptor low density lipoprotein receptor-related protein 3 |
| Family LDLLRP | LRP5 | Receptor Low density lipoprotein receptor-related protein 5 |
| Family LDLLRP | LRP6 | Receptor LDL receptor-related protein 6 |
| Family LDLLRP | LRP8 | Receptor low density lipoprotein receptor-related protein 8, |
| | | apolipoprotein e receptor |
| Family LDLLRP | SORL1 | Receptor sortilin-related receptor, L(DLR class) A repeats-containing |
| Family LDLLRP | VLDLR | Receptor Very low-density lipoprotein receptor precursor |
| Family Netrin\|Netrin1 | UNC5A | Receptor Netrin Receptor KIAA1976 protein |
| Family Netrin\|Netrin1 | UNC5B | Receptor Netrin Receptor p53-regulated receptor for death and life |
| Family Netrin\|Netrin2 | RASSF8 | Receptor Netrin Receptor Similar to unc5 homolog |
| Family Netrin\|Netrin2 | UNC5A | Receptor Netrin Receptor Similar to transmembrane receptor Unc5H1- |
| | | Fragment |
| Family Netrin\|Netrin2 | UNC5C | Receptor Netrin Receptor Transmembrane receptor UNC5C |
| Family Netrin\|Netrin2 | UNC5D | Receptor Netrin Receptor Hypothetical protein KIAA1777 |
| Family Neurexins | CNTNAP1 | Receptor CONTACTIN-ASSOCIATED PROTEIN 1 |
| Family Other | ARTS-1 | Receptor type 1 tumor necrosis factor receptor shedding aminopeptidase |
| | | regulator |
| Family Other | ATP6AP2 | Receptor renin receptor |
| Family Other | CD300LB | Receptor triggering receptor expressed on myeloid cells 5 |
| Family Other | CD36 | Receptor CD36 antigen (collagen type I receptor, thrombospondin |
| | | receptor) |
| Family Other | FCGR3A | Receptor Fc fragment of IgG, low affinity IIIa, receptor (CD16a) |
| Family Other | FCGR3B | Receptor Fc fragment of IgG, low affinity IIIb, receptor (CD16b) |
| Family Other | HMMR | Receptor hyaluronan-mediated motility receptor (RHAMM) |
| Family Other | LANCL1 | Receptor LanC lantibiotic synthetase component C-like 1 (bacterial) |
| Family Other | NGFRAP1 | Receptor nerve growth factor receptor (TNFRSF16) associated protein 1 |
| | | isoform a |
| Family Other | OGFR | Receptor OPIOID GROWTH FACTOR RECEPTOR |
| Family Other | OPRS1 | Receptor opioid receptor, sigma 1 isoform 1 |
| Family Other | PLAUR | Receptor plasminogen activator, urokinase receptor |
| Family Other | PROCR | Receptor protein C receptor, endothelial (EPCR) |
| Family Other | RELL1 | Receptor RELL1 |
| Family Other | RTN4R | Receptor Reticulon 4 receptor [Precursor] |
| Family Other | SCARF1 | Receptor scavenger receptor class F, member 1 isoform 1 |
| Family Other | SEZ6L2 | Receptor type I transmembrane receptor (seizure-related protein) |
| Family Other | SORT1 | Receptor sortilin 1 |
| Family Other | TGFBR3 | Receptor transforming growth factor, beta receptor III (betaglycan, |
| | | 300 kDa) |
| Family Other | TREM1 | Receptor Triggering-receptor TREM1 |
| Family Other | TREM2 | Receptor triggering receptor expressed on myeloid cells 2 |
| Family Other\|Folate | FOLR1 | Receptor FOLATE RECEPTOR 1 |
| Family Other\|Folate | FOLR2 | Receptor FOLATE RECEPTOR 2 |
| Family Other\|Folate | FOLR3 | Receptor FOLATE RECEPTOR 3 |
| Family Other\|Immune-Cell- | NCR1 | Receptor natural cytotoxicity triggering receptor 1 |
| R\|NatCytotoxicTriggR | | |
| Family Other\|Immune-Cell- | NCR2 | Receptor natural cytotoxicity triggering receptor 2 |
| R\|NatCytotoxicTriggR | | |
| Family Other\|Immune-Cell- | NCR3 | Receptor natural cytotoxicity triggering receptor 3 |
| R\|NatCytotoxicTriggR | | |
| Family Other\|Immune-Cell-R\|Nectin | PVR | Receptor poliovirus receptor |
| Family Other\|Immune-Cell-R\|Nectin | PVRL1 | Receptor poliovirus receptor-related 1 (herpesvirus entry mediator C; |
| | | nectin) isoform 1 |
| Family Other\|Immune-Cell-R\|Nectin | PVRL2 | Receptor poliovirus receptor-related 2 (herpesvirus entry mediator B) |
| Family Other\|Immune-Cell-R\|Nectin | PVRL3 | Receptor poliovirus receptor-related 3 |
| Family Other\|Immune-Cell-R\|Nectin | PVRL4 | Receptor poliovirus receptor-related 4 |
| Family Other\|Immune-Cell- | KIR2DL1 | Receptor Killer cell immunoglobulin-like receptor 2DL1 precursor |
| R\|NK_Cell_IG-like_R | | |
| Family Other\|Immune-Cell- | KIR2DL2 | Receptor Killer cell immunoglobulin-like receptor 2DL2 precursor |
| R\|NK_Cell_IG-like_R | | |
| Family Other\|Immune-Cell- | KIR2DL3 | Receptor Killer cell immunoglobulin-like receptor 2DL3 precursor |
| R\|NK_Cell_IG-like_R | | |
| Family Other\|Immune-Cell- | KIR2DL4 | Receptor Killer cell immunoglobulin-like receptor 2DL4 precursor |
| R\|NK_Cell_IG-like_R | | |
| Family Other Immune-Cell- | KIR2DS1 | Receptor Killer cell immunoglobulin-like receptor 2DS1 precursor |
| R\|NK_Cell_IG-like_R | | |
| Family Other\|Immune-Cell- | KIR2DS2 | Receptor Killer cell immunoglobulin-like receptor 2DS2 precursor |
| R\|NK_Cell_IG-like_R | | |
| Family Other\|Immune-Cell- | KIR2DS3 | Receptor Killer cell immunoglobulin-like receptor 2DS3 precursor |
| R\|NK_Cell_IG-like_R | | |
| Family Other\|Immune-Cell- | KIR2DS4 | Receptor Killer cell immunoglobulin-like receptor 2DS4 precursor |
| R\|NK_Cell_IG-like_R | | |
| Family Other\|Immune-Cell- | KIR2DS5 | Receptor Killer cell immunoglobulin-like receptor 2DS5 precursor |
| R\|NK_Cell_IG-like_R | | |
| Family Other\|Immune-Cell- | KIR3DL1 | Receptor Killer cell immunoglobulin-like receptor 3DL1 precursor |
| R\|NK_Cell_IG-like_R | | |
| Family Other\|Immune-Cell- | KIR3DL2 | Receptor Killer cell immunoglobulin-like receptor 3DL2 precursor |
| R\|NK_Cell_IG-like_R | | |
| Family Other\|Immune-Cell- | KIR3DS1 | Receptor Killer cell immunoglobulin-like receptor 3DS1 precursor |
| R\|NK_Cell_IG-like_R | | |
| Family Other\|Immune-Cell- | KLRA1 | Receptor killer cell lectin-like receptor subfamily A, member 1 |
| R\|NK_cell_lectin-likeR | | |
| Family Other\|Immune-Cell- | KLRC1 | Receptor killer cell lectin-like receptor subfamily C, member 1 |
| R\|NK_cell_lectin-likeR | | |
| Family Other\|Immune-Cell- | KLRC2 | Receptor killer cell lectin-like receptor subfamily C, member 2 |
| R\|NK_cell_lectin-likeR | | |
| Family Other\|Immune-Cell- | KLRC3 | Receptor killer cell lectin-like receptor subfamily C, member 3 |
| R\|NK_cell_lectin-likeR | | |
| Family Other\|Immune-Cell- | KLRC4 | Receptor killer cell lectin-like receptor subfamily C, member 4 |
| R\|NK_cell_lectin-likeR | | |
| Family Other\|Immune-Cell- | KLRD1 | Receptor killer cell lectin-like receptor subfamily D, member 1 |
| R\|NK_cell_lectin-likeR | | |
| Family Other\|Immune-Cell- | KLRE1 | Receptor killer cell lectin-like receptor family E member 1 |
| R\|NK_cell_lectin-likeR | | |
| Family Other\|Immune-Cell- | KLRG1 | Receptor killer cell lectin-like receptor subfamily G, member 1 |
| R\|NK_cell_lectin-likeR | | |
| Family Other\|Immune-Cell- | KLRH1 | Receptor killer cell lectin-like receptor subfamily H, member 1 |
| R\|NK_cell_lectin-likeR | | |
| Family Other\|Immune-Cell- | KLRI1 | Receptor killer cell lectin-like receptor subfamily I, member 1 |
| R\|NK_cell_lectin-likeR | | |
| Family Other\|Immune-Cell- | KLRK1 | Receptor killer cell lectin-like receptor subfamily K, member 1 |
| R\|NK_cell_lectin-likeR | | |
| Family Other\|Immune-Cell-R\|T-cell | CD3G | Receptor T-cell surface glycoprotein CD3 gamma chain precursor |
| Family Other\|Immune-Cell-R\|T- | T cell receptor alpha chain | Receptor T cell receptor alpha chain |
| cell\|Alpha | | |
| Family Other\|Immune-Cell-R\|T- | Similar to T cell receptor beta locus | Receptor Similar to T cell receptor beta locus |
| cell\|Beta | | |
| Family Other\|Immune-Cell-R\|T- | T cell receptor beta chain | Receptor T cell receptor beta chain |
| cell\|Beta | | |
| Family Other\|Immune-Cell-R\|T- | T-cell receptor beta 2 chain | Receptor T-cell receptor beta 2 chain |
| cell\|Beta | | |
| Family Other\|Immune-Cell-R\|T- | T-cell receptor beta chain | Receptor T-cell receptor beta chain |
| cell\|Beta | | |
| Family Other\|Immune-Cell-R\|T- | TRDD3 | Receptor T cell receptor delta diversity 3 |
| cell\|Delta | | |
| Family Other\|Immune-Cell-R\|T- | T cell receptor gamma chain variable g4 | Receptor T cell receptor gamma chain variable g4 |
| cell\|Gamma | | |
| Family Other\|Immune-Cell-R\|T- | T-cell receptor g2 | Receptor T-cell receptor g2 |
| cell\|Gamma | | |
| Family Other\|Immune-Cell-R\|T- | T-cell receptor gamma chain precursor g3 | Receptor T-cell receptor gamma chain precursor g3 |
| cell\|Gamma | | |
| Family Other\|Immune-Cell-R\|T- | T-cell receptor gamma chain precursor g5 | Receptor T-cell receptor gamma chain precursor g5 |
| cell\|Gamma | | |
| Family Other\|Immune-Cell-R\|T- | TRG@ | Receptor T-CELL ANTIGEN RECEPTOR, GAMMA SUBUNIT |
| cell\|Gamma | | |
| Family Other\|LeukocyteIg-likeR | LEUKOCYTE IMMUNOGLOBULIN-LIKE | Receptor LEUKOCYTE IMMUNOGLOBULIN-LIKE RECEPTOR 9 |
| | RECEPTOR 9 | |
| Family Other\|LeukocyteIg-likeR | LILRA1 | Receptor LEUKOCYTE IMMUNOGLOBULIN-LIKE RECEPTOR, |
| | | SUBFAMILY A, MEMBER 1 |
| Family Other\|LeukocyteIg-likeR | LILRA3 | Receptor LEUKOCYTE IMMUNOGLOBULIN-LIKE RECEPTOR, |
| | | SUBFAMILY A, MEMBER 3 |
| Family Other\|LeukocyteIg-likeR | LILRB1 | Receptor LEUKOCYTE IMMUNOGLOBULIN-LIKE RECEPTOR, |
| | | SUBFAMILY B, MEMBER 1 |
| Family Other\|LeukocyteIg-likeR | LILRB2 | Receptor LEUKOCYTE IMMUNOGLOBULIN-LIKE RECEPTOR, |
| | | SUBFAMILY B, MEMBER 2 |
| Family Other\|LeukocyteIg-likeR | LILRB3 | Receptor LEUKOCYTE IMMUNOGLOBULIN-LIKE RECEPTOR, |
| | | SUBFAMILY B, MEMBER 3 |
| Family Other\|LeukocyteIg-likeR | LILRB4 | Receptor LEUKOCYTE IMMUNOGLOBULIN-LIKE RECEPTOR, |
| | | SUBFAMILY B, MEMBER 4 |
| Family Other\|LeukocyteIg-likeR | LILRB5 | Receptor LEUKOCYTE IMMUNOGLOBULIN-LIKE RECEPTOR, |
| | | SUBFAMILY B, MEMBER 5 |
| Family Other\|Misc | KREMEN1 | Receptor kringle containing transmembrane protein 1 |
| Family Other\|Misc | KREMEN2 | Receptor Kringle-containing transmembrane protein precursor |
| Family Other\|Misc | PTDSR | Receptor Phosphatidylserine receptor beta |
| Family Other\|Neuropilins | NRP1 | Receptor Neuropilin 1 |
| Family Other\|Neuropilins | NRP2 | Receptor Neuropilin 2 |
| Family Other\|Phagocytosis | CD93 | Receptor Complement component C1q receptor [Precursor] |
| Family Other\|RAMP | RAMP1 | Receptor RECEPTOR ACTIVITY-MODIFYING PROTEIN 1 |
| Family Other\|RAMP | RAMP2 | Receptor RECEPTOR ACTIVITY-MODIFYING PROTEIN 2 |
| Family Other\|RAMP | RAMP3 | Receptor RECEPTOR ACTIVITY-MODIFYING PROTEIN 3 |
| Family Other\|Scavenger | SCARB1 | Receptor scavenger receptor class B, member 1 |
| Family Other\|Scavenger\|c-lectinn- | ASGR1 | Receptor Asialoglycoprotein receptor 1 |
| containing | | |
| Family Other\|Scavenger\|c-lectinn- | ASGR2 | Receptor Asialoglycoprotein receptor 2 |
| containing | | |
| Family Other\|Scavenger\|c-lectinn- | CLEC1B | Receptor C-type lectin-like receptor-2 |
| containing | | |
| Family Other\|Scavenger\|c-lectinn- | CLEC2D | Receptor Lectin-like NK cell receptor LLT1 |
| containing | | |
| Family Other\|Scavenger\|c-lectinn- | COLEC12 | Receptor collectin sub-family member 12 isoform I |
| containing | | |
| Family Other\|Scavenger\|c-lectinn- | FCER2 | Receptor Low affinity immunoglobulin epsilon FC receptor |
| containing | | |
| Family Other\|Scavenger\|c-lectinn- | KLRF1 | Receptor Lectin-like receptor F1 |
| containing | | |
| Family Other\|Scavenger\|c-lectinn- | LLR-G1 | Receptor Similar to killer cell lectin-like receptor subfamily G |
| containing | | |
| Family Other\|Scavenger\|c-lectinn- | OLR1 | Receptor Lectin-like OXIDIZED LDL receptor |
| containing | | |
| Family Other\|Scavenger\|Mannose | IGF2R | Receptor insulin-like growth factor 2 receptor |
| Family Other\|Scavenger\|Mannose | MRC1 | Receptor Macrophage mannose receptor precursor |
| Family Other\|Scavenger\|Mannose | MRC2 | Receptor Urokinase receptor-associated protein UPARAP |
| Family Other\|Scavenger\|Mannose | PLA2R1 | Receptor 180 kDa transmembrane PLA2 receptor |
| Family Other\|Selectin | SELL | Receptor SELECTIN L |
| Family Other\|Transferrin | TFR2 | Receptor TRANSFERRIN RECEPTOR 2 |
| Family Other\|Transferrin | TFRC | Receptor TRANSFERRIN RECEPTOR |
| Family Plexins | PLXNC1 | Receptor Plexin C1 |
| Family Roundabout | ROBO3 | Receptor roundabout, axon guidance receptor, homolog 3 (Drosophila) |
| Family RPTP | PTPRA | Receptor protein tyrosine phosphatase, receptor type, A isoform 1 |
| | | precursor |
| Family RPTP | PTPRD | Receptor protein tyrosine phosphatase, receptor type, D isoform 1 |
| | | precursor |
| Family RPTP | PTPRE | Receptor protein tyrosine phosphatase, receptor type, E isoform 1 |
| | | precursor |
| Family RPTP | PTPRF | Receptor protein tyrosine phosphatase, receptor type F isoform 2 |
| Family RPTP | PTPRS | Receptor protein tyrosine phosphatase, receptor type, S |
| Family RPTP\|RPTPETA | PTPRH | Receptor Protein-tyrosine phosphatase, receptor-type, H precursor |
| Family RPTP\|RPTPETA | PTPRO | Receptor receptor-type protein tyrosine phosphatase O, isoform b |
| | | precursor |
| Family RPTP\|RPTPOIC | PTPRN | Receptor Protein-tyrosine phosphatase-like N precursor SMART |
| Family RPTP\|RPTPOIC | PTPRN2 | Receptor protein tyrosine phosphatase, receptor type, N polypeptide 2, |
| | | isoform 2 precursor |
| Family RPTP\|RPTPOIC | PTPRO | Receptor receptor-type protein tyrosine phosphatase O, isoform c |
| | | precursor |
| Family RPTP\|RPTPOIC | PTPRS | Receptor protein tyrosine phosphatase, receptor type, S |
| Family RPTP\|RPTPUI5 | PTPRF | Receptor protein tyrosine phosphatase, receptor type, F, isoform 1 |
| Family RPTP\|RPTPUI5 | PTPRU | Receptor protein tyrosine phosphatase |
| Family RTK | ALK | Receptor ALK tyrosine kinase receptor precursor |
| Family RTK | LTK | Receptor Leukocyte tyrosine kinase receptor precursor |
| Family RTK | MUSK | Receptor muscle, skeletal, receptor tyrosine kinase |
| Family RTK | RET | Receptor Proto-oncogene tyrosine-protein kinase receptor ret precursor |
| Family RTK | TEK | Receptor Angiopoietin 1 receptor precursor |
| Family RTK | TIE1 | Receptor Tyrosine-protein kinase receptor Tie-1 precursor |
| Family RTK\|DDR | DDR1 | Receptor Epithelial discoidin domain receptor 1 precursor |
| Family RTK\|DDR | DDR2 | Receptor Discoidin domain receptor 2 precursor |
| Family RTK\|EPHRIN | EPHA1 | Receptor Ephrin type-A receptor 1 precursor |
| Family RTK\|EPHRIN | EPHA2 | Receptor Ephrin type-A receptor 2 precursor |
| Family RTK\|EPHRIN | EPHA3 | Receptor Ephrin type-A receptor 3 precursor |
| Family RTK\|EPHRIN | EPHA4 | Receptor Ephrin type-A receptor 4 precursor |
| Family RTK\|EPHRIN | EPHA5 | Receptor Ephrin type-A receptor 5 precursor |
| Family RTK\|EPHRIN | EPHA6 | Receptor EPHRIN RECEPTOR EphA6 |
| Family RTK\|EPHRIN | EPHA7 | Receptor Ephrin type-A receptor 7 precursor |
| Family RTK\|EPHRIN | EPHB1 | Receptor Ephrin type-B receptor 1 precursor |
| Family RTK\|EPHRIN | EPHB2 | Receptor Ephrin type-B receptor 2 precursor |
| Family RTK\|EPHRIN | EPHB3 | Receptor Ephrin type-B receptor 3 precursor |
| Family RTK\|EPHRIN | EPHB4 | Receptor ephrin receptor EphB4 |
| Family RTK\|EPHRIN | EPHB6 | Receptor Ephrin type-B receptor 6 precursor |
| Family RTK\|EPHRIN | similar to Eph receptor A6 | Receptor similar to Eph receptor A6 |
| Family RTK\|ERBB/EGF | EGFR | Receptor Epidermal growth factor receptor precursor |
| Family RTK\|ERBB/EGF | ERBB2 | Receptor Receptor protein-tyrosine kinase erbB-2 precursor |
| Family RTK\|ERBB/EGF | ERBB3 | Receptor Receptor protein-tyrosine kinase erbB-3 precursor |
| Family RTK\|ERBB/EGF | ERBB4 | Receptor Receptor protein-tyrosine kinase erbB-4 Precursor |
| Family RTK\|FGR | FGFR1 | Receptor fibroblast growth factor receptor 1 isoform 1 precursor |
| Family RTK\|FGR | FGFR1 | Receptor fibroblast growth factor receptor 1 isoform 2 precursor |
| Family RTK\|FGR | FGFR1 | Receptor fibroblast growth factor receptor 1 isoform 3 precursor |
| Family RTK\|FGR | FGFR1 | Receptor fibroblast growth factor receptor 1 isoform 8 precursor |
| Family RTK\|FGR | FGFR1 | Receptor fibroblast growth factor receptor 4 isoform 2 precursor |
| Family RTK\|FGR | FGFR1 | Receptor fibroblast growth factor receptor 5 isoform 2 precursor |
| Family RTK\|FGR | FGFR1 | Receptor fibroblast growth factor receptor 6 isoform 2 precursor |
| Family RTK\|FGR | FGFR1 | Receptor fibroblast growth factor receptor 7 isoform 2 precursor |
| Family RTK\|FGR | FGFR1 | Receptor fibroblast growth factor receptor 9 isoform 2 precursor |
| Family RTK\|FGR | FGFR1 | Receptor Heparin-binding growth factor receptor |
| Family RTK\|FGR | FGFR2 | Receptor FIBROBLAST GROWTH FACTOR RECEPTOR 2 |
| Family RTK\|FGR | FGFR3 | Receptor Fibroblast growth factor receptor 3 precursor |
| Family RTK\|FGR | FGFR4 | Receptor Fibroblast growth factor receptor 4 precursor |
| Family RTK\|FGR | FGFRL1 | Receptor fibroblast growth factor receptor-like 1 |
| Family RTK\|INSULIN-R | IGF1R | Receptor Insulin-like growth factor I receptor precursor |
| Family RTK\|INSULIN-R | INSR | Receptor Insulin receptor precursor |
| Family RTK\|INSULIN-R | INSRR | Receptor Insulin receptor-related protein precursor |
| Family RTK\|MET | MET | Receptor Hepatocyte growth factor receptor [Precursor] |
| Family RTK\|MET | MST1R | Receptor Macrophage-stimulating protein receptor precursor |
| Family RTK\|NGFR/NTR/TRK | NTRK1 | Receptor neurotrophic tyrosine kinase, receptor, type 1 |
| Family RTK\|NGFR/NTR/TRK | NTRK2 | Receptor neurotrophic tyrosine kinase, receptor, type 2 |
| Family RTK\|NGFR/NTR/TRK | NTRK3 | Receptor TRKC protein |
| Family RTK\|ROR | ROR1 | Receptor Tyrosine-protein kinase transmembrane receptor ROR1 |
| | | precursor |
| Family RTK\|ROR | ROR2 | Receptor Tyrosine-protein kinase transmembrane receptor ROR2 |
| | | precursor |
| Family RTK\|TYR/MER/UFO | AXL | Receptor Tyrosine-protein kinase receptor UFO precursor |
| Family RTK\|TYR/MER/UFO | MERTK | Receptor Proto-oncogene tyrosine-protein kinase MER precursor |
| Family RTK\|TYR/MER/UFO | TYRO3 | Receptor Tyrosine-protein kinase receptor TYRO3 precursor |
| Family RTK\|VEGF/PDGF | FLT1 | Receptor Vascular endothelial growth factor receptor 1 precursor |
| Family RTK\|VEGF/PDGF | FLT3 | Receptor FL cytokine receptor precursor |
| Family RTK\|VEGF/PDGF | FLT4 | Receptor Vascular endothelial growth factor receptor 3 precursor |
| Family RTK\|VEGF/PDGF | KDR | Receptor Vascular endothelial growth factor receptor 2 precursor |
| Family RTK\|VEGF/PDGF | KIT | Receptor Mast/stem cell growth factor receptor precursor |
| Family RTK\|VEGF/PDGF | PDGFRA | Receptor Alpha platelet-derived growth factor receptor precursor |
| Family RTK\|VEGF/PDGF | PDGFRB | Receptor Beta platelet-derived growth factor receptor precursor |
| Family SerThrK\|ALK | ACVR1 | Receptor Activin receptor type I |
| Family SerThrK\|ALK | ACVR1B | Receptor Serine/threonine-protein kinase receptor R2 |
| Family SerThrK\|ALK | ACVR1C | Receptor Activin receptor-like kinase 7 |
| Family SerThrK\|ALK | ACVRL1 | Receptor Serine/threonine-protein kinase receptor R3 precursor |
| Family SerThrK\|ALK | BMPR1A | Receptor Bone morphogenetic protein receptor type IA precursor |
| Family SerThrK\|ALK | BMPR1B | Receptor Bone morphogenetic protein receptor type IB precursor |
| Family SerThrK\|ALK | TGFBR1 | Receptor TGF-beta receptor type I precursor |
| Family SerThrK\|Type 2 receptor | ACVR2A | Receptor Activin receptor type II precursor |
| Family SerThrK\|Type 2 receptor | ACVR2B | Receptor Activin receptor type IIB |
| Family SerThrK\|Type 2 receptor | AMHR2 | Receptor Anti-mullerian hormone type II receptor precursor |
| Family SerThrK\|Type 2 receptor | BMPR2 | Receptor Bone morphogenetic protein receptor type II precursor |
| Family SerThrK\|Type 2 receptor | TGFBR2 | Receptor TGF-beta receptor type II precursor |
| Family Tetraspanins | RDS | Receptor Peripherin |
| Family TNFNGF | EDAR | Receptor Tumor necrosis factor receptor superfamily member EDAR |
| Family TNFNGF | LTBR | Receptor lymphotoxin beta receptor |
| Family TNFNGF | TNFRSF12A | Receptor Tumor necrosis factor receptor superfamily member Fn14 |
| Family TNFNGF | TNFRSF19L | Receptor Tumor necrosis factor receptor superfamily member 19L |
| Family TNFNGF | TNFRSF1B | Receptor Tumor necrosis factor receptor superfamily member 1B |
| Family TNFNGF | TNFRSF22 | Receptor tumor necrosis factor receptor superfamily, member 22 |
| Family TNFNGF | TRAF2 | Receptor TNF receptor-associated factor 2 |
| Family TNFNGF | TRAF3 | Receptor TNF receptor-associated factor 3 |
| Family TNFNGF | TRAF5 | Receptor TNF receptor-associated factor 5 |
| Family TNFNGF\|T13C | TNFRSF13C | Receptor Tumor necrosis factor receptor superfamily member 13C |
| Family TNFNGF\|T13C | TNFRSF17 | Receptor Tumor necrosis factor receptor superfamily member 17 (B-cell |
| | | maturation protein) |
| Family TNFNGF\|TNR4 | CD40 | Receptor Tumor necrosis factor receptor superfamily member 5 precursor |
| Family TNFNGF\|TNR4 | FAS | Receptor Tumor necrosis factor receptor superfamily member 6 precursor |
| Family TNFNGF\|TNR4 | TNFRSF14 | Receptor Tumor necrosis factor receptor superfamily, member 14 |
| | | precursor |
| Family TNFNGF\|TNR4 | TNFRSF4 | Receptor Tumor necrosis factor receptor superfamily member 4 precursor |
| Family TNFNGF\|TNR4 | TNFRSF7 | Receptor Tumor necrosis factor receptor superfamily, member 7 |
| | | precursor |
| Family TNFNGF\|TNR8 | TNFRSF11A | Receptor Tumor necrosis factor receptor superfamily member 11A |
| | | precursor |
| Family TNFNGF\|TNR8 | TNFRSF11B | Receptor Tumor necrosis factor receptor superfamily member 11B |
| | | precursor |
| Family TNFNGF\|TNR8 | TNFRSF21 | Receptor Tumor necrosis factor receptor superfamily member 21 |
| | | precursor |
| Family TNFNGF\|TNR8 | TNFRSF6B | Receptor Tumor necrosis factor receptor superfamily member 6B |
| | | precursor |
| Family TNFNGF\|TNR8 | TNFRSF8 | Receptor Tumor necrosis factor receptor superfamily member 8 precursor |
| Family TNFNGF\|TNR9 | TNFRSF13B | Receptor Tumor necrosis factor receptor superfamily member 13B |
| Family TNFNGF\|TNR9 | TNFRSF9 | Receptor Tumor necrosis factor receptor superfamily member 9 precursor |
| Family TNFNGF\|TR12 | TNFRSF1A | Receptor Tumor necrosis factor receptor superfamily member 1A |
| | | precursor |
| Family TNFNGF\|TR12 | TNFRSF25 | Receptor Tumor necrosis factor receptor superfamily member 12 |
| | | [Precursor] |
| Family TNFNGF\|TR16 | EDA2R | Receptor Tumor necrosis factor receptor superfamily member XEDAR |
| Family TNFNGF\|TR16 | NGFR | Receptor Tumor necrosis factor receptor superfamily member 16 |
| | | precursor |
| Family TNFNGF\|TR16 | TNFRSF18 | Receptor tumor necrosis factor receptor superfamily, member 18 |
| Family TNFNGF\|TR16 | TNFRSF19 | Receptor Tumor necrosis factor receptor superfamily member 19 |
| | | precursor |
| Family TNFNGF\|TRAIL | TNFRSF10A | Receptor Tumor necrosis factor receptor superfamily member 10A |
| | | precursor |
| Family TNFNGF\|TRAIL | TNFRSF10B | Receptor Tumor necrosis factor receptor superfamily member 10B |
| | | precursor |
| Family TNFNGF\|TRAIL | TNFRSF10C | Receptor Tumor necrosis factor receptor superfamily member 10C |
| | | precursor |
| Family TNFNGF\|TRAIL | TNFRSF10D | Receptor Tumor necrosis factor receptor superfamily member 10D |
| | | precursor |
| Family Toll\|TIL | TLR1 | Receptor Toll-like receptor 1 precursor |
| Family Toll\|TIL | TLR10 | Receptor Toll-like receptor 10 precursor |
| Family Toll\|TIL | TLR2 | Receptor Toll-like receptor 2 precursor |
| Family Toll\|TIL | TLR4 | Receptor Toll-like receptor 4 precursor |
| Family Toll\|TIL | TLR6 | Receptor Toll-like receptor 6 precursor |
| Family Toll\|TLR9 | TLR3 | Receptor Toll-like receptor 3 precursor |
| Family Toll\|TLR9 | TLR5 | Receptor Toll-like receptor 5 precursor |
| Family Toll\|TLR9 | TLR7 | Receptor Toll-like receptor 7 precursor |
| Family Toll\|TLR9 | TLR8 | Receptor Toll-like receptor 8 precursor |
| Family Toll\|TLR9 | TLR9 | Receptor Toll-like receptor 9 precursor |

APPENDIX B

| Ligand Family | Gene | Ligand |
|---|---|---|
| 7TM\|A\|Polypeptide\|Apelin/angiotensin/ | APLN | Ligand Apelin 13 |
| bradykinin | | |
| 7TM\|A\|Polypeptide\|Apelin/angiotensin/ | APLN | Ligand Apelin-28 |
| bradykinin | | |
| 7TM\|A\|Polypeptide\|Apelin/angiotensin/ | APLN | Ligand Apelin-31 |
| bradykinin | | |
| 7TM\|A\|Polypeptide\|Apelin/angiotensin/ | APLN | Ligand Apelin-36 |
| bradykinin | | |
| 7TM\|A\|Polypeptide\|Apelin/angiotensin/ | KNG1 | Ligand bradykinin |
| bradykinin | | |
| 7TM\|A\|Polypeptide\|Gonadotropin- | GNRH1 | Ligand GONADOTROPIN-RELEASING HORMONE 1 |
| releasing | | |
| 7TM\|A\|Polypeptide\|LGR (glycoprotein | INSL3 | Ligand INSULIN-LIKE 3 |
| hormones, relaxin-like) | | |
| 7TM\|A\|Polypeptide\|LGR (glycoprotein | INSL6 | Ligand INSULIN-LIKE 6 |
| hormones, relaxin-like) | | |
| 7TM\|A\|Polypeptide\|PROKINETICIN | PROK1 | Ligand Prokineticin 1 |
| 7TM\|A\|Polypeptide\|Proteinase-activated/ | F2 | Ligand coagulation factor II (thrombin) |
| thrombin | | |
| 7TM\|A\|Polypeptide\|Somatostatin&OPRL | NPB | Ligand neuropeptide B |
| 7TM\|A\|Polypeptide\|Somatostatin&OPRL | NPW | Ligand neuropeptide W |
| 7TM\|B\|Orphan\|CD97 | CD55 | Ligand Decay-accelerating Factor For Complement |
| 7TM\|B\|Polypeptide | CRH | Ligand CORTICOTROPIN-RELEASING HORMONE |
| 7TM\|B\|Polypeptide | GHRH | Ligand GROWTH HORMONE-RELEASING HORMONE |
| ADAMs | ADAM17 | Ligand a disintegrin and metalloproteinase domain 17 (tumor necrosis factor, alpha, |
| | | converting enzyme) |
| Angiopoietin | ANGPTL4 | Ligand ANGIOPOIETIN-LIKE 4 |
| Chemokines | CX3CL1 | Ligand chemokine (C—X3—C motif) ligand 1 |
| Chemokines | XCL1 | Ligand chemokine (C motif) ligand 1 |
| Chemokines\|CCL | CCL1 | Ligand chemokine (C-C motif) ligand 1 |
| Chemokines\|CCL | CCL11 | Ligand chemokine (C-C motif) ligand 11 |
| Chemokines\|CCL | CCL12 | Ligand chemokine (C-C motif) ligand 12 |
| Chemokines\|CCL | CCL13 | Ligand chemokine (C-C motif) ligand 13 |
| Chemokines\|CCL | CCL14 | Ligand chemokine (C-C motif) ligand 14 |
| Chemokines\|CCL | CCL15 | Ligand chemokine (C-C motif) ligand 15 |
| Chemokines\|CCL | CCL16 | Ligand chemokine (C-C motif) ligand 16 |
| Chemokines\|CCL | CCL17 | Ligand chemokine (C-C motif) ligand 17 |
| Chemokines\|CCL | CCL18 | Ligand chemokine (C-C motif) ligand 18 (pulmonary and activation-regulated) |
| Chemokines\|CCL | CCL19 | Ligand chemokine (C-C motif) ligand 19 |
| Chemokines\|CCL | CCL20 | Ligand chemokine (C-C motif) ligand 20 |
| Chemokines\|CCL | CCL21 | Ligand chemokine (C-C motif) ligand 21 |
| Chemokines\|CCL | CCL22 | Ligand chemokine (C-C motif) ligand 22 |
| Chemokines\|CCL | CCL23 | Ligand chemokine (C-C motif) ligand 23 |
| Chemokines\|CCL | CCL24 | Ligand chemokine (C-C motif) ligand 24 |
| Chemokines\|CCL | CCL25 | Ligand chemokine (C-C motif) ligand 25 |
| Chemokines\|CCL | CCL26 | Ligand chemokine (C-C motif) ligand 26 |
| Chemokines\|CCL | CCL27 | Ligand Chemokine, Cc Motif, Ligand 27 |
| Chemokines\|CCL | CCL28 | Ligand chemokine (C-C motif) ligand 28 |
| Chemokines\|CCL | CCL3 | Ligand chemokine (C-C motif) ligand 3 |
| Chemokines\|CCL | CCL4 | Ligand chemokine (C-C motif) ligand 4 |
| Chemokines\|CCL | CCL5 | Ligand chemokine (C-C motif) ligand 5 |
| Chemokines\|CCL | CCL6 | Ligand chemokine (C-C motif) ligand 6 |
| Chemokines\|CCL | CCL7 | Ligand chemokine (C-C motif) ligand 7 |
| Chemokines\|CCL | CCL8 | Ligand chemokine (C-C motif) ligand 8 |
| Chemokines\|CXCL | CCL2 | Ligand Small Inducible Cytokine A2 |
| Chemokines\|CXCL | CXCL1 | Ligand chemokine (C—X—C motif) ligand 1 (melanoma growth stimulating activity, |
| | | alpha) |
| Chemokines\|CXCL | CXCL10 | Ligand Chemokine, Cxc Motif, Ligand 10 |
| Chemokines\|CXCL | CXCL11 | Ligand chemokine (C—X—C motif) ligand 11 |
| Chemokines\|CXCL | CXCL12 | Ligand Chemokine, Cxc Motif, Ligand 12, Isoform 1 |
| Chemokines\|CXCL | CXCL12 | Ligand Chemokine, Cxc Motif, Ligand 12, Isoform 2 |
| Chemokines\|CXCL | CXCL13 | Ligand chemokine (C—X—C motif) ligand 13 (B-cell chemoattractant) |
| Chemokines\|CXCL | CXCL14 | Ligand chemokine (C—X—C motif) ligand 14 |
| Chemokines\|CXCL | CXCL15 | Ligand chemokine (C—X—C motif) ligand 15 |
| Chemokines\|CXCL | CXCL16 | Ligand chemokine (C—X—C motif) ligand 16 |
| Chemokines\|CXCL | CXCL2 | Ligand chemokine (C—X—C motif) ligand 2 |
| Chemokines\|CXCL | CXCL3 | Ligand chemokine (C—X—C motif) ligand 3 |
| Chemokines\|CXCL | CXCL5 | Ligand chemokine (C—X—C motif) ligand 5 |
| Chemokines\|CXCL | CXCL6 | Ligand chemokine (C—X—C motif) ligand 6 (granulocyte chemotactic protein 2) |
| Chemokines\|CXCL | CXCL9 | Ligand chemokine (C—X—C motif) ligand 9 |
| Chemokines\|CXCL | IL8 | Ligand interleukin 8 |
| Chemokines\|CXCL | PF4 | Ligand platelet factor 4 (chemokine (C—X—C motif) ligand 4) |
| Chemokines\|CXCL | PPBP | Ligand pro-platelet basic protein (chemokine (C—X—C motif) ligand 7) |
| Complement | CFH | Ligand complement factor H |
| CystineKnotPL\|GDNF | GDNF | Ligand Glial Cell Line-derived Neurotrophic Factor |
| CystineKnotPL\|GDNF | ARTN | Ligand ARTEMIN |
| CystineKnotPL\|GDNF | PSPN | Ligand Persephin |
| CystineKnotPL\|GDNF | NRTN | Ligand Neurturin |
| CystineKnotPL\|PDGF | FIGF | Ligand c-fos induced growth factor (vascular endothelial growth factor D) |
| CystineKnotPL\|PDGF | PDGFA | Ligand platelet-derived growth factor alpha polypeptide |
| CystineKnotPL\|PDGF | PDGFB | Ligand Platelet-derived Growth Factor, Beta Polypeptide |
| CystineKnotPL\|PDGF | PDGFC | Ligand Platelet-derived Growth Factor C |
| CystineKnotPL\|PDGF | PDGFD | Ligand platelet derived growth factor D |
| CystineKnotPL\|PDGF | VEGF | Ligand VASCULAR ENDOTHELIAL GROWTH FACTOR |
| CystineKnotPL\|PDGF | VEGFC | Ligand vascular endothelial growth factor C |
| CystineKnotPL\|SlitLike | SLIT1 | Ligand slit homolog 1 (Drosophila) |
| CystineKnotPL\|SlitLike | SLIT3 | Ligand slit homolog 3 |
| CystineKnotPL\|TGF-betaFamL | AMH | Ligand ANTI-MULLERIAN HORMONE |
| CystineKnotPL\|TGF-betaFamL | BMP15 | Ligand Bone Morphogenetic Protein 15 |
| CystineKnotPL\|TGF-betaFamL | BMP2 | Ligand Bone Morphogenetic Protein 2 |
| CystineKnotPL\|TGF-betaFamL | BMP3 | Ligand Bone Morphogenetic Protein 3 |
| CystineKnotPL\|TGF-betaFamL | BMP4 | Ligand Bone Morphogenetic Protein 4 |
| CystineKnotPL\|TGF-betaFamL | BMP5 | Ligand Bone Morphogenetic Protein 5 |
| CystineKnotPL\|TGF-betaFamL | BMP6 | Ligand Bone Morphogenetic Protein 6 |
| CystineKnotPL\|TGF-betaFamL | BMP7 | Ligand Bone Morphogenetic Protein 7 |
| CystineKnotPL\|TGF-betaFamL | BMP8 | Ligand Bone Morphogenetic Protein 8 |
| CystineKnotPL\|TGF-betaFamL | BMP9 | Ligand Bone Morphogenetic Protein 9 |
| CystineKnotPL\|TGF-betaFamL | BMP10 | Ligand Bone Morphogenetic Protein 10 |
| CystineKnotPL\|TGF-betaFamL | BMP11 | Ligand Bone Morphogenetic Protein 11 |
| CystineKnotPL\|TGF-betaFamL | GDF1 | Ligand growth differentiation factor 1 |
| CystineKnotPL\|TGF-betaFamL | GDF10 | Ligand growth differentiation factor 10 |
| CystineKnotPL\|TGF-betaFamL | GDF11 | Ligand growth differentiation factor 11 |
| CystineKnotPL\|TGF-betaFamL | GDF15 | Ligand growth differentiation factor 15 |
| CystineKnotPL\|TGF-betaFamL | GDF2 | Ligand growth differentiation factor 2 |
| CystineKnotPL\|TGF-betaFamL | GDF3 | Ligand growth differentiation factor 3 |
| CystineKnotPL\|TGF-betaFamL | GDF5 | Ligand growth differentiation factor 5 (cartilage derived morphogenetic protein-1) |
| CystineKnotPL\|TGF-betaFamL | GDF6 | Ligand growth differentiation factor 6 |
| CystineKnotPL\|TGF-betaFamL | GDF7 | Ligand growth differentiation factor 7 |
| CystineKnotPL\|TGF-betaFamL | GDF8 | Ligand growth differentiation factor 8 |
| CystineKnotPL\|TGF-betaFamL | GDF9 | Ligand growth differentiation factor 9 |
| CystineKnotPL\|TGF-betaFamL | Nodal | Ligand Nodal |
| CystineKnotPL\|TGF-betaFamL | INHA | Ligand INHIBIN, ALPHA |
| CystineKnotPL\|TGF-betaFamL | INHBA | Ligand INHIBIN, BETA A |
| CystineKnotPL\|TGF-betaFamL | INHBB | Ligand INHIBIN, BETA B |
| CystineKnotPL\|TGF-betaFamL | INHBC | Ligand INHIBIN, BETA C |
| CystineKnotPL\|TGF-betaFamL | TGFB1 | Ligand Transforming Growth Factor, Beta-1 |
| CystineKnotPL\|TGF-betaFamL | TGFB2 | Ligand transforming growth factor, beta 2 |
| CystineKnotPL\|TGF-betaFamL | TGFB3 | Ligand Transforming growth factor, beta 3 |
| Cytokine1R | CNTF | Ligand CILIARY NEUROTROPHIC FACTOR |
| EGFfamL | AREG | Ligand amphiregulin (schwannoma-derived growth factor) |
| EGFfamL | EGF | Ligand EPIDERMAL GROWTH FACTOR |
| EGFfamL | HBEGF | Ligand heparin-binding EGF-like growth factor |
| EGFfamL | TDGF1 | Ligand TERATOCARCINOMA-DERIVED GROWTH FACTOR 1 |
| EGFfamL | TGFA | Ligand transforming growth factor, alpha |
| EphrinL | EFNB1 | Ligand Ephrin B1 |
| FGF | FGF1 | Ligand fibroblast growth factor 1 (acidic) isoform 3 precursor |
| FGF | FGF10 | Ligand fibroblast growth factor 10 |
| FGF | FGF11 | Ligand fibroblast growth factor 11 |
| FGF | FGF12 | Ligand fibroblast growth factor 12 |
| FGF | FGF13 | Ligand fibroblast growth factor 13 |
| FGF | FGF14 | Ligand fibroblast growth factor 14 |
| FGF | FGF16 | Ligand fibroblast growth factor 16 |
| FGF | FGF17 | Ligand fibroblast growth factor 17 |
| FGF | FGF18 | Ligand fibroblast growth factor 18 |
| FGF | FGF19 | Ligand fibroblast growth factor 19 |
| FGF | FGF2 | Ligand fibroblast Growth Factor 2 |
| FGF | FGF20 | Ligand fibroblast growth factor 20 |
| FGF | FGF21 | Ligand fibroblast growth factor 21 |
| FGF | FGF22 | Ligand fibroblast growth factor 22 |
| FGF | FGF23 | Ligand fibroblast growth factor 23 |
| FGF | FGF3 | Ligand fibroblast growth factor 3 precursor |
| FGF | FGF4 | Ligand fibroblast growth factor 4 precursor |
| FGF | FGF5 | Ligand fibroblast growth factor 5 |
| FGF | FGF6 | Ligand fibroblast growth factor 6 |
| FGF | FGF7 | Ligand fibroblast growth factor 7 precursor |
| FGF | FGF8 | Ligand fibroblast Growth Factor 8 |
| FGF | FGF9 | Ligand fibroblast growth factor 9 precursor |
| InsFamL | IGF1 | Ligand INSULIN-LIKE GROWTH FACTOR I |
| InsFamL | IGF2 | Ligand insulin-like growth factor 2 (somatomedin A) |
| Interleukin ligands | IL10 | Ligand Interleukin 10 |
| Interleukin ligands | IL11 | Ligand interleukin 11 |
| Interleukin ligands | IL12A | Ligand interleukin 12A (natural killer cell stimulatory factor 1, cytotoxic lymphocyte |
| | | maturation factor 1, p35) |
| Interleukin ligands | IL12B | Ligand interleukin 12B (natural killer cell stimulatory factor 2, cytotoxic lymphocyte |
| | | maturation factor 2, p40) |
| Interleukin ligands | IL13 | Ligand interleukin 13 |
| Interleukin ligands | IL15 | Ligand interleukin 15 |
| Interleukin ligands | IL17A | Ligand Interleukin 17 |
| Interleukin ligands | IL17B | Ligand interleukin 17B |
| Interleukin ligands | IL18 | Ligand Interleukin 18 |
| Interleukin ligands | IL19 | Ligand interleukin 19 |
| Interleukin ligands | IL1A | Ligand Interleukin 1-alpha |
| Interleukin ligands | IL1B | Ligand Interleukin 1-beta |
| Interleukin ligands | IL2 | Ligand interleukin 2 |
| Interleukin ligands | IL20 | Ligand interleukin 20 |
| Interleukin ligands | IL21 | Ligand interleukin 21 |
| Interleukin ligands | IL22 | Ligand interleukin 22 |
| Interleukin ligands | IL23A | Ligand interleukin 23, alpha subunit p19 |
| Interleukin ligands | IL24 | Ligand interleukin 24 |
| Interleukin ligands | IL25 | Ligand interleukin 25 |
| Interleukin ligands | IL28A | Ligand interleukin 28A (interferon, lambda 2) |
| Interleukin ligands | IL28B | Ligand interleukin 28B (interferon, lambda 3) |
| Interleukin ligands | IL29 | Ligand interleukin 29 (interferon, lambda 1) |
| Interleukin ligands | IL3 | Ligand interleukin 3 (colony-stimulating factor, multiple) |
| Interleukin ligands | IL31 | Ligand interleukin 31 |
| Interleukin ligands | IL4 | Ligand interleukin 4 |
| Interleukin ligands | IL5 | Ligand interleukin 5 (colony-stimulating factor, eosinophil) |
| Interleukin ligands | IL6 | Ligand Interleukin 6 |
| Interleukin ligands | IL7 | Ligand Interleukin 7 |
| Interleukin ligands | IL9 | Ligand interleukin 9 |
| LigandOTHER | AGGF1 | Ligand angiogenic factor with G patch and FHA domains 1 |
| LigandOTHER | CLCF1 | Ligand cardiotrophin-like cytokine factor 1 |
| LigandOTHER | CRHBP | Ligand corticotropin releasing hormone binding protein |
| LigandOTHER | CTGF | Ligand connective tissue growth factor |
| LigandOTHER | CYR61 | Ligand cysteine-rich, angiogenic inducer, 61 |
| LigandOTHER | F13A1 | Ligand FACTOR XIII, A1 SUBUNIT |
| LigandOTHER | F7 | Ligand Coagulation Factor 7, activated, Isoform B |
| LigandOTHER | F8 | Ligand coagulation factor VIII, procoagulant component (hemophilia A) |
| LigandOTHER | F9 | Ligand HEMOPHILIA B |
| LigandOTHER | FLT3LG | Ligand fms-related tyrosine kinase 3 ligand |
| LigandOTHER | HDGF | Ligand hepatoma-derived growth factor (high-mobility group protein 1-like) |
| LigandOTHER | HGF | Ligand Hepatocyte Growth Factor |
| LigandOTHER | IGFALS | Ligand INSULIN-LIKE GROWTH FACTOR-BINDING PROTEIN, ACID- |
| | | LABILE SUBUNIT |
| LigandOTHER | IGFBP1 | Ligand INSULIN-LIKE GROWTH FACTOR-BINDING PROTEIN 1 |
| LigandOTHER | IGFBP2 | Ligand INSULIN-LIKE GROWTH FACTOR-BINDING PROTEIN 2 |
| LigandOTHER | IPF1 | Ligand INSULIN PROMOTER FACTOR 1 |
| LigandOTHER | KNG1 | Ligand kininogen 1 |
| LigandOTHER | LEFTY2 | Ligand left-right determination factor 2 |
| LigandOTHER | LIF | Ligand Leukemia-inhibitory Factor |
| LigandOTHER | MDK | Ligand midkine (neurite growth-promoting factor 2) |
| LigandOTHER | MIF | Ligand MACROPHAGE MIGRATION INHIBITORY FACTOR |
| LigandOTHER | MST1 | Ligand macrophage stimulating 1 (hepatocyte growth factor-like) |
| LigandOTHER | NGFB | Ligand NERVE GROWTH FACTOR, BETA SUBUNIT |
| LigandOTHER | PBEF1 | Ligand pre-B-cell colony enhancing factor 1 |
| LigandOTHER | PROC | Ligand protein C (inactivator of coagulation factors Va and VIIIa) |
| LigandOTHER | PTN | Ligand pleiotrophin (heparin binding growth factor 8, neurite growth-promoting |
| | | factor 1) |
| LigandOTHER | SELPLG | Ligand selectin P ligand |
| LigandOTHER | TFPI | Ligand tissue factor pathway inhibitor (lipoprotein-associated coagulation inhibitor) |
| LigandOTHER | THPO | Ligand thrombopoietin (myeloproliferative leukemia virus oncogene ligand, |
| | | megakaryocyte growth and development factor) |
| LigandOTHER | TITF1 | Ligand THYROID TRANSCRIPTION FACTOR 1 |
| LigandOTHER | VTN | Ligand vitronectin (serum spreading factor, somatomedin B, complement S-protein) |
| LigandOTHER | VWF | Ligand von Willebrand factor |
| NatriureticPept | NPPA | Ligand NATRIURETIC PEPTIDE PRECURSOR A |
| Neuregulin | NRG1 | Ligand neuregulin 1 |
| RTK\|NGFR/NTR/TRK | BDNF | Ligand Brain-derived Neurotrophic Factor |
| RTK\|VEGF/PDGF | CSF1 | Ligand Colony-stimulating Factor 1 |
| RTK\|VEGF/PDGF | CSF2 | Ligand Granulocyte-macrophage colony-stimulating factor |
| Ser-Cys_prot-ase_inhib | SERPINE2 | Ligand serine (or cysteine) proteinase inhibitor, clade E (nexin, plasminogen |
| | | activator inhibitor type 1), member 2 |
| TNF ligands\|TNSF7 | TNFSF12 | Ligand tumor necrosis factor (ligand) superfamily, member 12 isoform 1 precursor |
| TNF ligands\|TNSF7 | TNFSF12 | Ligand tumor necrosis factor (ligand) superfamily, member 12 isoform 2 |
| TNF ligands\|TNSF7 | TNFSF12-TNFSF13 | Ligand tumor necrosis factor (ligand) superfamily, member 12-member 13 |
| TNF ligands\|TNSF7 | TNFSF13 | Ligand tumor necrosis factor (ligand) superfamily, member 13 |
| TNF ligands\|TNSF7 | TNFSF13B | Ligand tumor necrosis factor (ligand) superfamily, member 13b |
| TNF ligands\|TNSF7 | TNFSF18 | Ligand tumor necrosis factor (ligand) superfamily, member 18 |
| TNF ligands\|TNSF7 | TNFSF4 | Ligand tumor necrosis factor (ligand) superfamily, member 4 (tax-transcriptionally |
| | | activated glycoprotein 1, 34 kDa) |
| TNF ligands\|TNSF7 | TNFSF7 | Ligand tumor necrosis factor (ligand) superfamily, member 7 |
| TNFNGF | CD40LG | Ligand CD40 ligand (TNF superfamily, member 5, hyper-IgM syndrome) |
| TNFNGF | FASLG | Ligand Fas Ligand |
| TNFNGF | GMFA | Ligand glia maturation factor, alpha |
| TNFNGF | KITLG | Ligand Kit Ligand |
| TNFNGF | TNF | Ligand tumor necrosis factor alpha |
| TNFNGF | TNFSF10 | Ligand tumor necrosis factor (ligand) superfamily, member 10 |
| TNFNGF | TNFSF11 | Ligand Tumor Necrosis Factor Ligand Superfamily, Member 11 |
| TNFNGF | TNFSF14 | Ligand tumor necrosis factor (ligand) superfamily, member 14 |
| TNFNGF | TNFSF15 | Ligand tumor necrosis factor (ligand) superfamily, member 15 |
| TNFNGF | TNFSF8 | Ligand tumor necrosis factor (ligand) superfamily, member 8 |
| TNFNGF | TNFSF9 | Ligand tumor necrosis factor (ligand) superfamily, member 9 |
| VEGFs | PGF | Ligand placental growth factor, vascular endothelial growth factor-related protein |

What is claimed is:

1. A method of ligand profiling of one or more distinct samples each comprising mixtures of ligand molecules, said method comprising:
a. contacting each of the distinct samples with one or more populations of receptor carriers, wherein each receptor carrier comprises a plurality of receptors to which the ligand molecules may bind;
b. washing unbound ligand molecules away and eluting bound ligand molecules from each population of the receptor carriers to provide separate ligand fractions; and
c. fractionating the ligand fractions to give separate profiles of ligand molecules for each of the distinct samples.

2. The method of claim 1, wherein each mixture of ligand molecules comprises one or more ligands with unknown identity or quantity.

3. The method of claim 1, wherein the one or more populations of receptor carriers are or are not different from each other.

4. The method of claim 1, wherein the receptor carriers are cells, a mixture of cells, organelles, cell ghost, cellular membranes, vesicles comprising a plurality of receptors, or artificial biological surface comprising a plurality of immobilized receptors.

5. The method of claim 4, wherein the cells or organelles are live or fixed.

6. The method of claim 4, wherein the cells express at least one exogenous receptor.

7. The method of claim 4, wherein the cells are treated with inhibitor of exocytosis or inhibitor of endocytosis.

8. The method of claim 4, wherein the cells are treated to get rid of cellular proteins loosely associated with cell membrane before contacting the cells with samples.

9. The method of claim 4, wherein the artificial biological surface is a surface of a culture well, a culture plate, a bead or a matrix.

10. The method of claim 4, wherein the artificial biological surface is made of nitrocellulose, cellulose, dextran, nylon, metal, plastic, latex, agarose, glass, or a silicon material.

11. The method of claim 1, wherein the receptors are cell surface polypeptides, secreted polypeptides, extracellular domains of receptors, nucleic acids, carbohydrates, lipids, organic molecules or inorganic molecules.

12. The method of claim 1, wherein the ligand molecules are polypeptides or non-polypeptide molecules.

13. The method of claim 1, wherein the sample is a biological fluid comprising culture supernatants, a cell lysate, or a bodily fluid of an organism.

14. The method of claim 13, wherein the bodily fluid is blood, blood plasma, blood serum, hemolysate, spinal fluid, urine, lymph, synovial fluid, saliva, semen, stool, sputum, tear, mucus, amniotic fluid, lacrimal fluid, cyst fluid, sweat gland secretion, milk, or bile.

15. The method of claim 1, wherein the sample is obtained from a normal individual, an individual with disease, or an individual undergoing treatment.

16. The method of claim 1, wherein fractionating the ligand fraction comprises detecting and quantifying multiple ligand molecules sequentially or simultaneously.

17. The method of claim 16, wherein the detection and quantification of ligand molecules comprise using mass spectrometry or antibodies.

18. The method of claim 1, wherein the ligand molecules are labeled with labeling molecules before or after contacting with the receptor carriers, wherein the labeling molecules can be detected directly or indirectly.

19. The method of claim 18, wherein the labeling molecules for the ligand molecules in one or more samples comprise fluorescence dyes.

20. The method of claim 18, wherein the labeling molecules comprise biotin, and are detected by detecting molecules selected from the group consisting of avidin, strepavidin, NeutrAvidin, and CapAvidin.

21. A kit for enriching multiple ligands from a sample comprising ligands with unknown identity or quantity, the kit comprising
a. a binding solution
b. a washing solution
c. an elution solution and
d. an instruction on experimental procedures according to the method of claim 1.
